(19) United States
(12) Patent Application Publication
(10) Pub. No.: US 2002/0077470 A1
(43) Pub. Date: Jun. 20, 2002

(54) Cardiac muscle-associated genes

(76) Inventors: Michael G. Walker, Sunnyvale, CA (US); Wayne Volkmuth, Calabasas, CA (US); Tod M. Klingler, San Carlos, CA (US); Yalda Azimzai, Castro Valley, CA (US)

Correspondence Address: INCYTE GENOMICS, INC., LEGAL DEPARTMENT, 3160 PORTER DRIVE, PALO ALTO, CA, 94304, US

(21) Appl. No.: 09/880,192

(22) Filed: Jun. 12, 2001

Related U.S. Application Data
(63) Continuation-in-part of application No. 09/299,708, filed on Apr. 26, 1999, now abandoned.

(51) Int. Cl. C12Q 1/68; C07H 21/04; C12P 21/02; C12N 5/06

(52) U.S. Cl. 536/24.3; 435/6; 435/69.1; 435/325; 435/320.1

(57) ABSTRACT

The invention provides compositions and novel polynucleotides and their encoded proteins that serve as surrogate markers in that they co-express with genes known to be involved associated with disorders associated with cardiac muscle function. The invention also provides expression vectors, host cells, proteins encoded by the polynucleotides and antibodies which specifically bind the proteins. The invention also provides methods for the diagnosis, prognosis, evaluation of therapies and treatment of disorders associated with cardiac muscle function.

This application is a continuation-in-part of U.S. Ser. No. 09/299,708, filed Apr. 26, 1999.

### FIELD OF THE INVENTION

The invention relates to 48 polynucleotides associated with cardiac muscle function that were identified by their coexpression with known cardiac muscle-associated genes. The invention also relates to the use of these polynucleotides, their encoded proteins and antibodies which specifically bind the proteins in diagnosis, prognosis, treatment, and evaluation of therapies for disorders associated with cardiac muscle function.

### BACKGROUND OF THE INVENTION

Vertebrates have three classes of muscle: skeletal, smooth, and cardiac. Skeletal and cardiac muscles have a striped appearance in the light microscope and are therefore called striated. Cardiac muscle resembles skeletal muscle in many respects, but it is specialized for the continuous, involuntary, rhythmic contractions needed for pumping blood. Smooth muscles lack striations and surround internal organs such as the intestines, the uterus, and large blood vessels. Skeletal muscle is under the voluntary control of the nervous system. Cardiac muscle and smooth muscle are under the involuntary control of the nervous system. Compared with striated muscles, smooth muscle cells contract and relax slowly and can create and maintain tension for long periods of time.

Muscle tissue is composed of bundles of multinucleated muscle cells (myofibers). Each muscle cell contains bundles of actin and myosin filaments (myofibrils) which extend the length of the cell. The myofibril is composed of a chain of sarcomeres. The sarcomere is the functional unit of contraction. Myosin filaments are sandwiched between alternating layers of actin filaments. Myosin filaments are composed of heavy and light chain proteins. Actin filaments are capped by two proteins, capZ and tropomodulin. In addition, the myosin-binding sites of actin filaments are protected by the tropomyosin-troponin regulatory complex. Contraction of muscle is initiated by action potential-stimulated release from the sarcoplasmic reticulum of calcium ions into the cell to levels greater than 10⁻⁶ M. Binding of calcium ions to troponin causes tropomyosin to move towards the center of the actin filament. This movement exposes the myosin-binding sites of actin. Prior to contraction, the N-terminal domain of the myosin heavy chain-light chain complex (myosin head) forms a cross-bridge with actin filaments. Binding of ATP to the myosin head causes dissociation of myosin from actin. This is followed by a conformational change of the myosin head and hydrolysis of ATP. The myosin head then forms a new cross-bridge with actin filaments. Successive cycle of ATP-binding, dissociation from actin, conformational changes, ATP hydrolysis, and crossbridge formation results in muscle contraction. Relaxation is initiated when calcium ion levels in the cell fall below 10⁻⁶ M. At that level, calcium ions dissociate from troponin, which then shields the myosin-binding sites of actin.

Gap junctions, very permeable parts of the cell membrane, connect individual muscle cells with each other. Through these gap junctions, ions diffuse relatively freely and transmit action potentials to all muscle cells.

Differentiation of muscle cells during embryogenesis and ontogeny is regulated by a number of nuclear transcription factors such as myogenin, MyoD, MEF2A, and myf-5, and by cell cycle proteins such as p21, p57, and RB. Expression of the genes which encode some of these myogenic regulatory proteins has been correlated with certain type of tumor and other disorders (Wang et al. (1995) Am J Pathol 147:1799-1810; Miyagawa et al.(1998) Nat Genet 18:15-17; and Sedehizade et al.(1997) Muscle Nerve 20:186-194).

Contemporary techniques for diagnosis of cardiac muscle abnormalities rely mainly on observation of clinical symptoms, electrocardiograms, and serological analyses of metabolites and enzymes. Relatively mild symptoms in the earlier stages of heart disease may even be overlooked. In addition, the serological analyses of the limited number of hormones or peptides do not always differentiate among those diseases or syndromes which have overlapping or near-normal ranges of hormonal or marker protein levels. Thus, development of new techniques, such as microarrays and transcript imaging, will contribute to the early and accurate diagnosis or to a better understanding of molecular pathogenesis of cardiac disorders.

The present invention satisfies a need in the art by providing new compositions that are useful for diagnosis, prognosis, treatment, and evaluation of therapies for disorders associated with cardiac muscle function.

### SUMMARY OF THE INVENTION

The invention provides a composition comprising a plurality of polynucleotides having the nucleic acid sequences of SEQ ID NOs:1-48 that are highly significantly co-expressed with known the cardiac muscle-associated genes: atrial regulatory myosin, ventricular myosin alkali light chain, cardiac troponin, cardiac ventricular myosin, cardiodilatin, creatine kinase M, myoglobin, natriuretic peptide precursor, sarcomeric mitochondrial creatine kinase, telethonin, titin, and urocortin.

The invention also provides an isolated polynucleotide comprising a nucleic acid sequence selected from SEQ ID NOs:1-48 and the complements thereof. In different aspects, the polynucleotide is used as a surrogate marker, as a probe, in an expression vector, and in the diagnosis, prognosis, evaluation of therapies and treatment of disorders such as atherosclerosis, arteriosclerosis, atrial fibrillation, cancer (myxoma) and complications of cancer, cardiac injury, congestive heart failure, coronary artery disease, hypertension, hypertrophic cardiomyopathy, myocardial hypertrophy, myocardial infarction, and plaque. The invention further provides a composition comprising a polynucleotide and a labeling moiety.

The invention provides a method for using a composition or a polynucleotide to screen a plurality of molecules and compounds to identify or to purify ligands which specifically bind to the composition or the polynucleotide. The molecules are selected from DNA molecules, RNA molecules, peptide nucleic acids, peptides, mimetics, ribozymes, transcription factors, enhancers, and repressors.

The invention provides a method for using a composition or a polynucleotide to detect gene expression in a sample by hybridizing the composition or polynucleotide to nucleic acids of the sample under conditions for formation of one or more hybridization complexes and detecting hybridization complex formation, wherein complex formation indicates gene expression in the sample. In one aspect, the composition or polynucleotide is attached to a substrate. In another aspect, the nucleic acids of the sample are amplified prior to hybridization. In yet another aspect, complex formation is compared with at least one standard and indicates the presence of a disorder.

The invention provides a purified protein or a portion thereof selected from SEQ ID NOs:49-62, which is encoded by a polynucleotide that is highly significantly co-expressed with genes known to involved in disorders associated with cardiac muscle function. The invention also provides a method for using a protein to screen a plurality of molecules to identify or to purify at least one ligand which specifically binds the protein. The molecules are selected from aptamers, DNA molecules, RNA molecules, peptide nucleic acids, peptides, mimetics, ribozymes, proteins, antibodies, agonists, antagonists, immunoglobulins, inhibitors, pharmaceutical agents or drug compounds.

The invention provides a method of using a protein to make an antibody comprising immunizing a animal with the protein under conditions to elicit an antibody response, isolating animal antibodies, attaching the protein to a substrate, contacting the substrate with isolated antibodies under conditions to allow specific binding to the protein, and dissociating the antibodies from the protein, thereby obtaining purified antibodies. The invention also provides a method for using the antibody to detect expression of a protein in a sample, the method comprising combining the antibody with a sample under conditions which allow the formation of antibody:protein complexes, and detecting complex formation, wherein complex formation indicates expression of the protein in the sample. The invention also provides a composition comprising a polynucleotide, a protein, or an antibody that specifically binds a protein and a labeling moiety or a pharmaceutical carrier.

### BRIEF DESCRIPTION OF THE SEQUENCE LISTING AND TABLES

The Sequence Listing provides exemplary polynucleotide sequences, SEQ ID NOs:1-48, and polypeptide sequences, SEQ ID NOs:49-62. Each sequence is identified by a sequence identification number (SEQ ID NO) and by the Incyte clone number with which the sequence was first identified.

Table 1 presents the results of co-expression analysis. The entries in the table are the p-values which link the novel polynucleotides with known marker genes.

Table 2 shows the characterization of proteins having the amino acid sequences of SEQ ID NO:49-62.

### DESCRIPTION OF THE INVENTION

It must be noted that as used herein and in the appended claims, the singular forns “a”, “an”, and “the” include the plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Definitions

“Markers” refer to polynucleotides, proteins, and antibodies which are useful in the diagnosis, prognosis, evaluation of therapies and treatment of disorders associated with cardiac muscle function. Typically, this means that the marker gene or polynucleotide is differentially expressed in samples from subjects predisposed to, manifesting, or diagnosed with disorders associated with cardiac muscle function.

“Differential expression” refers to an increased or up-regulated or a decreased or down-regulated expression as detected by presence, absence or at least about a two-fold change in the amount of transcribed messenger RNA or protein in a sample.

“Disorders associated with cardiac muscle function” specifically include, but are not limited to, the following conditions, diseases, and disorders: atherosclerosis, arteriosclerosis, atrial fibrillation, cancer (myxoma) and complications of cancer, cardiac injury, congestive heart failure, coronary artery disease, hypertension, hypertrophic cardiomyopathy, myocardial hypertrophy, myocardial infarction, and plaque.

“Isolated or purified” refers to a polynucleotide or protein that is removed from its natural environment and that is separated from other components with which it is naturally present.

“Genes known to be highly, and differentially, expressed in cardiac muscle function” which were used in the co-expression analysis included atrial regulatory myosin, ventricular myosin alkali light chain, cardiac troponin, cardiac ventricular myosin, cardiodilatin, creatine kinase M, myoglobin, natriuretic peptide precursor, sarcomeric mitochondrial creatine kinase, telethonin, titin, and urocortin.

“Polynucleotide” refers to an isolated cDNA. It can be of genomic or synthetic origin, double-stranded or single-stranded, and combined with vitamins, minerals, carbohydrates, lipids, proteins, or other nucleic acids to perform a particular activity or form a useful composition.

“Protein” refers to a purified polypeptide whether naturally occurring or synthetic.

“Sample” is used in its broadest sense. A sample containing nucleic acids can comprise a bodily fluid; an extract from a cell; a chromosome, organelle, or membrane isolated from a cell; genomic DNA, RNA, or cDNA in solution or bound to a substrate; a cell; a tissue; a tissue print; and the like.

“Substrate” refers to any rigid or semi-rigid support to which polynucleotides or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” is a profile of gene transcription activity in a particular tissue at a particular time.

A “variant” refers to a polynucleotide or protein whose sequence diverges from about 5% to about 30% from the nucleic acid or amino acid sequences of the Sequence Listing.

The Invention

The present invention employed “guilt by association (GBA)”, a method for using marker genes known to be associated with cardiac muscle function to identify surrogate markers, polynucleotides that are similarly associated or co-expressed in the same tissues, pathways or disorders (Walker and Volkmuth (1999) Prediction of gene function by genome-scale expression analysis: prostate-associated genes. Genome Res 9:1198-1203, incorporated herein by reference). The genes known to be associated with cardiac muscle function are atrial regulatory myosin, ventricular myosin alkali light chain, cardiac troponin, cardiac ventricular myosin, cardiodilatin, creatine kinase M, myoglobin, natriuretic peptide precursor, sarcomeric mitochondrial creatine kinase, telethonin, titin, and urocortin. In particular, the method identifies cDNAs cloned from mRNA transcripts which were active in tissues removed from subjects with cardiac disorders including, but not limited to, atherosclerosis, arteriosclerosis, atrial fibrillation, cancer (myxoma) and complications of cancer, cardiac injury, congestive heart failure, coronary artery disease, hypertension, hypertrophic cardiomyopathy, myocardial hypertrophy, myocardial infarction, and plaque. The polynucleotides, their encoded proteins and antibodies which specifically bind to the encoded proteins are useful in the diagnosis, prognosis, evaluation of therapies, and treatment of disorders associated with cardiac muscle function. U.S. Ser. No. 09/299,708 is incorporated in its entirety by reference herein.

Guilt by association provides for the identification of polynucleotides that are expressed in a plurality of libraries. The polynucleotides represent genes of unknown function which are co-expressed in a specific pathway, disease process, subcellular compartment, cell type, tissue, or species. The expression patterns of the genes known to be highly and differentially expressed during cardiac muscle function; atrial regulatory myosin, ventricular myosin alkali light chain, cardiac troponin, cardiac ventricular myosin, cardiodilatin, creatine kinase M, myoglobin, natriuretic peptide precursor, sarcomeric mitochondrial creatine kinase, telethonin, titin, and urocortin; are compared with those of polynucleotides with unknown function to determine whether a specified co-expression probability threshold is met. Through this comparison, a subset of the polynucleotides having a high co-expression probability with the known marker genes can be identified.

The polynucleotides originate from human cDNA libraries. These polynucleotides can also be selected from a variety of sequence types including, but not limited to, expressed sequence tags (ESTs), assembled polynucleotides, full length coding regions, and 3′ untranslated regions. To be considered in GBA or co-expression analysis, the polynucleotides had to have been expressed in at least five cDNA libraries. In this application, GBA was applied to a total of 45,233 assembled polynucleotide bins that met the criteria of having been expressed in at least five libraries.

The cDNA libraries used in the co-expression analysis were obtained from adrenal gland, biliary tract, bladder, blood cells, blood vessels, bone marrow, brain, bronchus, cartilage, chromaffin system, colon, connective tissue, cultured cells, embryonic stem cells, endocrine glands, epithelium, esophagus, fetus, ganglia, heart, hypothalamus, hemic/immune system, intestine, islets of Langerhans, kidney, larynx, liver, lung, lymph, muscles, neurons, ovary, pancreas, penis, phagocytes, pituitary, placenta, pleura, prostate, salivary glands, seminal vesicles, skeleton, spleen, stomach, testis, thymus, tongue, ureter, uterus, and the like. The number of cDNA libraries analyzed can range from as few as three to greater than 10,000 and preferably, the number of the cDNA libraries is greater than 500.

In a preferred embodiment, the polynucleotides are assembled from related sequences, such as sequence fragments derived from a single transcript. Assembly of the polynucleotide can be performed using sequences of various types including, but not limited to, ESTs, extension of the EST, shotgun sequences from a cloned insert, or full length cDNAs. In a most preferred embodiment, the polynucleotides are derived from human sequences that have been assembled using the algorithm disclosed in U.S. Ser. No. 9,276,534, filed Mar. 25, 1999, and used in U.S. Ser. No. 09/226,994, filed Jan. 7, 1999, both incorporated herein by reference.

Experimentally, differential expression of the polynucleotides can be evaluated by methods including, but not limited to, differential display by spatial immobilization or by gel electrophoresis, genome mismatch scanning, representational difference analysis, and transcript imaging. For example, the results of transcript imaging for SEQ ID NOs:29 and 44 are shown in Example IX. Differential expression of SEQ ID NO:29 is highly specifically correlated with hypertension, and SEQ ID NO:44, with myocardial infarction. The transcript image provided direct confirmation of the strength of co-expression analysis--the use of known genes to identify unknown polynucleotides and their encoded proteins which are highly significantly associated with disorders associated with cardiac muscle function. Additionally, differential expression can be assessed by microarray technology. These methods can be used alone or in combination.

Genes known to be highly expressed in disorders associated with cardiac muscle function can be selected based on research in which the genes are found to be key elements of biochemical or signaling pathways or on the known use of the genes as diagnostic or prognostic markers or therapeutic targets for such disorders. Preferably, the known genes are atrial regulatory myosin, ventricular myosin alkali light chain, cardiac troponin, cardiac ventricular myosin, cardiodilatin, creatine kinase M, myoglobin, natriuretic peptide precursor, sarcomeric mitochondrial creatine kinase, telethonin, titin, and urocortin.

The procedure for identifying novel polynucleotides that exhibit a statistically significant co-expression pattern with known genes is as follows. First, the presence or absence of a polynucleotide in a cDNA library is defined: a polynucleotide is present in a cDNA library when at least one cDNA fragment corresponding to the polynucleotide is detected in a cDNA from that library, and a polynucleotide is absent from a library when no corresponding cDNA fragment is detected.

Second, the significance of co-expression is evaluated using a probability method to measure a due-to-chance probability of the co-expression. The probability method can be the Fisher exact test, the chi-squared test, or the kappa test. These tests and examples of their applications are well known in the art and can be found in standard statistics texts (Agresti (1990) Categorical Data Analysis, John Wiley & Sons, New York N.Y.; Rice (1988) Mathematical Statistics and Data Analysis, Duxbury Press, Pacific Grove Calif.). A Bonferroni correction (Rice, supra, p. 384) can also be applied in combination with one of the probability methods for correcting statistical results of one polynucleotide versus multiple other polynucleotides. In a preferred embodiment, the due-to-chance probability is measured by a Fisher exact test, and the threshold of the due-to-chance probability is set preferably to less than 0.001, more preferably to less than 0.00001.

For example, to determine whether two genes, A and B, have similar co-expression patterns, occurrence data vectors can be generated as illustrated in the table below. The presence of a gene occurring at least once in a library is indicated by a one, and its absence from the library, by a zero.

| | Library 1 | Library 2 | Library 3 | . . . | Library N |
|---|---|---|---|---|---|
| Gene A | 1 | 1 | 0 | . . . | 0 |
| Gene B | 1 | 0 | 1 | . . . | 0 |

For a given pair of genes, the occurrence data in the table above can be summarized in a 2×2 contingency table. The second table (below) presents co-occurrence data for gene A and gene B in a total of 30 libraries. Both gene A and gene B occur 10 times in the libraries.

| | Gene A Present | Gene A Absent | Total |
|---|---|---|---|
| Gene B Present | 8 | 2 | 10 |
| Gene B Absent | 2 | 18 | 20 |
| Total | 10 | 20 | 30 |

The second table summarizes and presents: 1) the number of times gene A and B are both present in a library; 2) the number of times gene A and B are both absent in a library; 3) the number of times gene A is present, and gene B is absent; and 4) the number of times gene B is present, and gene A is absent. The upper left entry is the number of times the two genes co-occur in a library, and the middle right entry is the number of times neither gene occurs in a library. The off diagonal entries are the number of times one gene occurs, and the other does not. Both A and B are present eight times and absent 18 times. Gene A is present, and gene B is absent, two times; and gene B is present, and gene A is absent, two times. The probability (“p-value”) that the above association occurs due to chance as calculated using a Fisher exact test is 0.0003.

This method of estimating the probability for co-expression makes several assumptions. The method assumes that the libraries are independent and are identically sampled. However, in practical situations, the selected cDNA libraries are not entirely independent, because more than one library can be obtained from a single subject or tissue. Nor are they entirely identically sampled, because different numbers of cDNAs can have been sequenced from each library. The number of cDNAs sequenced typically ranges from 5,000 to 10,000 cDNAs per library. After the Fisher exact co-expression probability is calculated for each polynucleotide versus all other assembled polynucleotides that occur, a Bonferroni correction for multiple statistical tests is applied.

Using the method of the present invention, we have identified polynucleotides, SEQ ID NOs:1-48 and their encoded proteins, SEQ ID NOs:49-62, that exhibit highly significant co-expression probability with known marker genes for disorders associated with cardiac muscle function. The results presented in Example VI show the direct associations among the novel polynucleotides and the known marker genes for disorders associated with cardiac muscle function. Therefore, by these associations, the novel polynucleotides are useful as surrogate markers for the co-expressed known markers in diagnosis, prognosis, evaluation of therapies and treatment of disorders associated with cardiac muscle function. Further, the proteins or peptides expressed from the novel polynucleotides are either potential therapeutics or targets for the identification and/or development of therapeutics.

In one embodiment, the present invention encompasses a composition comprising a plurality of polynucleotides having the nucleic acid sequences of SEQ ID NOs:1-48 or the complements thereof. These 48 polynucleotides are shown by the method to have significant co-expression with known markers for disorders associated with cardiac muscle function. The invention also provides a polynucleotide, its complement, a probe comprising the polynucleotide or the complement thereof selected from SEQ ID NOs:1-48.

The polynucleotide can be used to search against the GenBank primate (pri), rodent (rod), mammalian (mam), vertebrate (vrtp), and eukaryote (eukp) databases; the encoded protein, against GenPept, SwissProt, BLOCKS (Bairoch et al. (1997) Nucleic Acids Res 25:217-221), PFAM, and other databases that contain previously identified and annotated protein sequences, motifs, and gene functions. Methods that search for primary sequence patterns with secondary structure gap penalties (Smith et al. (1992) Protein Engineering 5:35-51) as well as algorithms such as Basic Local Alignment Search Tool (BLAST; Altschul (1993) J Mol Evol 36:290-300; Altschul et al. (1990) J Mol Biol 215:403410), BLOCKS (Henikoff and Henikoff (1991) Nucleic Acids Res 19:6565-6572), Hidden Markov Models (HMM; Eddy (1996) Cur Opin Str Biol 6:361-365; Sonnhammer et al. (1997) Proteins 28:405-420), and the like, can be used to manipulate and analyze nucleotide and amino acid sequences. These databases, algorithms and other methods are well known in the art and are described in Ausubel et al. (1997; Short Protocols in Molecular Biology, John Wiley & Sons, New York N.Y., unit 7.7) and in Meyers (1995; Molecular Biology and Biotechnology, Wiley VCH, New York N.Y., p 856-853).

Also encompassed by the invention are polynucleotides that are capable of hybridizing to SEQ ID NOs:1-48 and the complements thereof under highly stringent conditions. Stringency can be defined by salt concentration, temperature, and other chemicals and conditions well known in the art. Conditions can be selected, for example, by varying the concentrations of salt in the prehybridization, hybridization, and wash solutions or by varying the hybridization and wash temperatures. With some substrates, the temperature can be decreased by adding a solvent such as formamide to the prehybridization and hybridization solutions.

Hybridization can be performed at low stringency, with buffers such as 5×SSC (saline sodium citrate) with 1% sodium dodecyl sulfate (SDS) at 60 C., which permits complex formation between two nucleic acid sequences that contain some mismatches. Subsequent washes are performed at higher stringency with buffers such as 0.2×SSC with 0.1% SDS at either 45 C. (medium stringency) or 68 C. (high stringency), to maintain hybridization of only those complexes that contain completely complementary sequences. Background signals can be reduced by the use of detergents such as SDS, sarcosyl, or TRITON X-100 (Sigma-Aldrich, St. Louis Mo.), and/or a blocking agent, such as salmon sperm DNA. Hybridization methods are described in detail in Ausubel (supra, units2.8-2.11, 3.18-3.19 and 4-6-4.9) and Sambrook et al. (1989; Molecular Cloning A Laboratory Manual, Cold Spring Harbor Press, Plainview N.Y.).

A polynucleotide can be extended utilizing primers and employing various PCR-based methods known in the art to detect upstream sequences such as promoters and other regulatory elements. (See, e.g., Dieffenbach and Dveksler (1995) PCR Primer, a Laboratory Manual, Cold Spring Harbor Press, Plainview N.Y.) Commercially available kits such as XL-PCR (Applied Biosystems (ABI), Foster City Calif.), cDNA libraries (Life Technologies, Rockville Md.) or genomic libraries (Clontech, Palo Alto Calif.) and nested primers can be used to extend the sequence. For all PCR-based methods, primers can be designed using commercially available software (e.g., LASERGENE software, DNASTAR, Madison Wis. or another program), to be about 15 to 30 nucleotides in length, to have a GC content of about 50%, and to form a hybridization complex at temperatures of about 68C. to 72C.

In another aspect of the invention, the polynucleotide can be cloned into a recombinant vector that directs the expression of the protein, or structural or functional portions thereof, in host cells. Due to the inherent degeneracy of the genetic code, other DNA sequences which encode functionally equivalent amino acid sequence can be produced and used to express the protein encoded by the polynucleotide. The nucleotide sequences of the present invention can be engineered using methods generally known in the art in order to alter the nucleotide sequences for a variety of purposes including, but not limited to, modification of the cloning, processing, and/or expression of the gene product. DNA shuffling by random fragmentation, as described in U.S. Pat. No. 5,830,721, and PCR reassembly of gene fragments and synthetic oligonucleotides can be used to engineer the nucleotide sequences. For example, oligonucleotide-mediated site-directed mutagenesis can be used to introduce mutations that create new restriction sites, alter glycosylation patterns, change codon preference, produce splice variants, and so forth.

In order to express a biologically active protein, the polynucleotide or derivatives thereof, can be inserted into an expression vector with elements for transcriptional and translational control of the inserted coding sequence in a particular host. These elements include regulatory sequences, such as enhancers, constitutive and inducible promoters, and 5′ and 3′ untranslated regions. Methods which are well known to those skilled in the art can be used to construct such expression vectors. These methods include in vitro recombinant DNA techniques, synthetic techniques, and in vivo genetic recombination (Ausubel, supra, unit 16).

A variety of expression vector/host cell systems can be utilized to express the polynucleotide. These include, but are not limited to, microorganisms such as bacteria transformed with recombinant bacteriophage, plasmid, or cosmid expression vectors; yeast transformed with yeast expression vectors; insect cell systems infected with baculovirus vectors; plant cell systems transformed with viral or bacterial expression vectors; or animal cell systems. For long term production of recombinant proteins in mammalian systems, stable expression in cell lines is preferred. For example, the polynucleotide can be transformed into cell lines using expression vectors which can contain viral origins of replication and/or endogenous expression elements and a selectable or visible marker gene on the same or on a separate vector. The invention is not to be limited by the vector or host cell employed.

In general, host cells that contain the polynucleotide and that express the protein can be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip-based technologies for the detection and/or quantification of nucleic acid or amino acid sequences. Immunological methods for detecting and measuring the expression of the protein using either specific polyclonal or monoclonal antibodies are known in the art. Examples of such techniques include enzyme-linked immunosorbent assays (ELISAs), radioimmunoassays (RIAs), and fluorescence activated cell sorting (FACS).

Host cells transformed with the polynucleotide can be cultured under conditions for the expression and recovery of the protein from cell culture. The protein produced by a transgenic cell can be secreted or retained intracellularly depending on the sequence and/or the vector used. As will be understood by those of skill in the art, expression vectors containing the polynucleotide can be designed to contain signal sequences which direct secretion of the protein through a prokaryotic cell wall or eukaryotic cell membrane.

In addition, a host cell strain can be chosen for its ability to modulate expression of the inserted sequences or to process the expressed protein in the desired fashion. Such modifications of the protein include, but are not limited to, acetylation, carboxylation, glycosylation, phosphorylation, lipidation, and acylation. Post-translational processing which cleaves a “prepro” form of the protein can also be used to specify protein targeting, folding, and/or activity. Different host cells which have specific cellular machinery and characteristic mechanisms for post-translational activities (e.g., CHO, HeLa, MDCK, HEK293, and WI38) are available from the ATCC (Manassas Va.) and can be chosen to ensure the correct modification and processing of the expressed protein.

In another embodiment of the invention, natural, modified, or recombinant polynucleotides are ligated to a heterologous sequence resulting in translation of a fusion protein containing heterologous protein moieties in any of the aforementioned host systems. Such heterologous protein moieties facilitate purification of fusion proteins using commercially available affinity matrices. Such moieties include, but are not limited to, glutathione S-transferase, maltose binding protein, thioredoxin, calmodulin binding peptide, 6-His, FLAG, c-myc, hemaglutinin, and monoclonal antibody epitopes.

In another embodiment, the polynucleotides, wholly or in part, are synthesized using chemical or enzymatic methods well known in the art (Caruthers et al. (1980) Nucl Acids Symp Ser (7) 215-233; Ausubel, supra, units 10.4 and 10.16). Peptide synthesis can be performed using various solid-phase techniques (Roberge et al. (1995) Science 269:202-204), and machines such as the ABI 431A peptide synthesizer (ABI) can be used to automate synthesis. If desired, the amino acid sequence can be altered during synthesis to produce a more stable variant for therapeutic use.

Screening, Diagnostics and Therapeutics

The polynucleotides can be used as surrogate markers in diagnosis, prognosis, evaluation of therapies and treatment of disorders associated with cardiac muscle function including, but not limited to, atherosclerosis, arteriosclerosis, atrial fibrillation, cancer (myxoma) and complications of cancer, cardiac injury, congestive heart failure, coronary artery disease, hypertension, hypertrophic cardiomyopathy, myocardial hypertrophy, myocardial infarction, and plaque.

The polynucleotide can be used to screen a plurality or library of molecules and compounds for specific binding affinity. The assay can be used to screen DNA molecules, RNA molecules, peptide nucleic acids, peptides, mimetics, ribozymes, or proteins including transcription factors, enhancers, repressors, and the like which regulate the activity of the polynucleotide in the biological system. The assay involves providing a plurality of molecules and compounds, combining a polynucleotide or a composition of the invention with the plurality of molecules and compounds under conditions to allow specific binding, and detecting specific binding to identify at least one molecule or compound which specifically binds at least one polynucleotides of the invention.

Similarly the proteins, or portions thereof, can be used to screen a plurality or library of molecules or compounds in any of a variety of screening assays to identify a ligand. The protein employed in such screening can be free in solution, affixed to an abiotic substrate or expressed on the external, or a particular internal surface, of a bacterial, or other, cell. Specific binding between the protein and the ligand can be measured. The assay can be used to screen aptamers, DNA molecules, RNA molecules, peptide nucleic acids, peptides, mimetics, ribozymes, proteins, antibodies, agonists, antagonists, immunoglobulins, inhibitors, pharmaceutical agents or drug compounds and the like, which specifically bind the protein. One method for high throughput screening using very small assay volumes and very small amounts of test compound is described in Burbaum et al. U.S. Pat. No. 5,876,946, incorporated herein by reference, which screens large numbers of molecules for enzyme inhibition or receptor binding.

In one preferred embodiment, the polynucleotides are used for diagnostic purposes to determine the differential expression of a gene in a sample. The polynucleotide consists of complementary RNA and DNA molecules, branched nucleic acids, and/or PNAs. In one alternative, the polynucleotides are used to detect and quantify gene expression in biopsied samples in which differential expression of the polynucleotide indicates the presence of a disorder. In another alternative, the polynucleotide can be used to detect genetic polymorphisms associated with a disease or disorder. In a preferred embodiment, these polymorphisms are detected in an mRNA transcribed from an endogenous gene.

In another preferred embodiment, the polynucleotide is used as a probe. Specificity of the probe is determined by whether it is made from a unique region, a regulatory region, or from a region encoding a conserved motif. Both probe specificity and the stringency of the diagnostic hybridization or amplification will determine whether the probe identifies only naturally occurring, exactly complementary sequences, allelic variants, or related sequences. Probes designed to detect related sequences should preferably have at least 50% sequence identity to at least a fragment of a polynucleotide of the invention.

Methods for producing hybridization probes include the cloning of nucleic acid sequences into vectors for the production of RNA probes. Such vectors are known in the art, are commercially available, and can be used to synthesize RNA probes in vitro by adding RNA polymerases and labeled nucleotides. Probes can incorporate nucleotides labeled by a variety of reporter groups including, but not limited to, radionuclides such as ³²P or ³⁵S, enzymatic labels such as alkaline phosphatase coupled to the probe via avidin/biotin coupling systems, fluorescent labels such as Cy3 and Cy5, and the like. The labeled polynucleotides can be used in Southern or northern analysis, dot blot, or other membrane-based technologies, on chips or other substrates, and in PCR technologies. Hybridization probes are also useful in mapping the naturally occurring genonic sequence. Fluorescent in situ hybridization (FISH) can be correlated with other physical chromosome mapping techniques and genetic map data as described in Heinz-ULrich et al. (In: Meyers, supra, pp. 965-968). In many cases, genomic context helps identify genes that encode a particular protein family. (See, e.g., Kirschning et al. (1997) Genomics 46:416-25.)

The polynucleotide can be labeled using standard methods and added to a sample from a subject under conditions for the formation and detection of hybridization complexes. After incubation the sample is washed, and the signal associated with complex formation is quantitated and compared with at least one standard value. Standard values are derived from any control sample, typically one that is free of the suspect disorder and from one that represents a single, specific and preferably, staged disorder. If the amount of signal in the subject sample is distinguishable from the standards, then differential expression in the subject sample indicates the presence of the disorder. Qualitative and quantitative methods for comparing complex formation in subject samples with previously established standards are well known in the art.

Such assays can also be used to evaluate the efficacy of a particular therapeutic treatment regimen in animal studies, in clinical trials, or to monitor the treatment of an individual subject. Once the presence of the disorder has been established and a treatment protocol is initiated, hybridization, amplification, or antibody assays can be repeated on a regular basis to determine when gene or protein expression in the patient begins to approximate that which is observed in a healthy subject. The results obtained from successive assays can be used to show the efficacy of treatment over a period ranging from several hours, e.g. in the case of toxic shock, to many years, e.g. in the case of osteoarthritis.

The polynucleotides can be used on a substrate such as a microarray to monitor gene expression, to identify splice variants, mutations, and polymorphisms. Information derived from analyses of expression patterns can be used to determine gene function, to understand the genetic basis of a disease, to diagnose a disorder, and to develop and monitor the activities of therapeutic agents used to treat a disorder. Microarrays can also be used to detect genetic diversity, single nucleotide polymorphisms, which may characterize a particular population, at the genomic level.

In another embodiment, antibodies or Fabs comprising an antigen binding site that specifically binds the protein can be used for the diagnosis of diseases characterized by the differential expression of the protein. A variety of protocols for measuring protein expression, including ELISAs, RIAs, FACS and antibody arrays, are well known in the art and provide a basis for diagnosing differential or abnormal levels of expression. Standard values for protein expression parallel those reviewed above for nucleotide expression. The amount of complex formation can be quantitated by various methods, preferably by photometric means. Quantities of the protein expressed in subject samples are compared with standard values. Deviation between standard and subject values establishes the parameters for diagnosing or monitoring a particular disorder. Alternatively, one can use competitive drug screening assays in which neutralizing antibodies capable of binding specifically with the protein compete with a test compound. Antibodies can be used to detect the presence of any peptide which shares one or more epitopes or antigenic determinants with the protein. In one aspect, the antibodies of the present invention can be used for treatment of a disorder, delivery of therapeutics, or monitoring therapy during treatment.

In another aspect, the polynucleotide, or its complement, can be used therapeutically for the purpose of expressing mRNA and protein, or conversely to block transcription or translation of the mRNA. Expression vectors can be constructed using elements from retroviruses, adenoviruses, herpes or vaccinia viruses, or bacterial plasmids, and the like. These vectors can be used for delivery of nucleotide sequences to a particular target cell population, tissue, or organ. Methods well known to those skilled in the art can be used to construct vectors to express the polynucleotides or their complements. (See, e.g., Maulik et al. (1997) Molecular Biotechnology, Therapeutic Applications and Strategies, Wiley-Liss, New York N.Y.)

Alternatively, the polynucleotide or its complement, can be used for somatic cell or stem cell gene therapy. Vectors can be introduced in vivo, in vitro, and ex vivo. For ex vivo therapy, vectors are introduced into stem cells taken from the subject, and the resulting transgenic cells are clonally propagated for autologous transplant back into that same subject. Delivery of the polynucleotide by transfection, liposome injections, or polycationic amino polymers can be achieved using methods which are well known in the art. (See, e.g., Goldman et al. (1997) Nature Biotechnology 15:462-466.) Additionally, endogenous gene expression can be inactivated using homologous recombination methods which insert an inactive gene sequence into the coding region or other targeted region of the genome. (See, e.g. Thomas et al. (1987) Cell 51: 503-512.)

Vectors containing the polynucleotide can be transformed into a cell or tissue to express a missing protein or to replace a nonfunctional protein. Similarly a vector constructed to express the complement of the polynucleotide can be transformed into a cell to down-regulate protein expression. Complementary or antisense sequences can consist of an oligonucleotide derived from the transcription initiation site; nucleotides between about positions −10 and +10 from the ATG are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee et al. In: Huber and Carr (1994) Molecular and Immunologic Approaches, Futura Publishing, Mt. Kisco N.Y., pp. 163-177.)

Ribozymes, enzymatic RNA molecules, can also be used to catalyze the cleavage of mRNA and decrease the levels of particular mRNAs, such as those comprising the polynucleotides of the invention. (See, e.g., Rossi (1994) Current Biology 4: 469-471.) Ribozymes can cleave MRNA at specific cleavage sites. Alternatively, ribozymes can cleave mRNAs at locations dictated by flanking regions that form complementary base pairs with the target mRNA. The construction and production of ribozymes is well known in the art and is described in Meyers (supra).

RNA molecules can be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiester linkages within the backbone of the molecule. Alternatively, nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases, can be included.

Further, an antagonist, or an antibody that binds specifically to the protein can be administered to a subject to treat a disorders associated with cardiac muscle function. The antagonist, antibody, or fragment can be used directly to inhibit the activity of the protein or indirectly to deliver a therapeutic agent to cells or tissues which express the protein. The therapeutic agent can be a cytotoxic agent selected from a group including, but not limited to, abrin, ricin, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin A and 40, radioisotopes, and glucocorticoid.

Antibodies to the protein can be generated using methods that are well known in the art. One method involves immunizing a animal with the protein selected from SEQ ID NOs:49-62 under conditions to elicit an antibody response; isolating animal antibodies; attaching the protein to a substrate; contacting the substrate with isolated antibodies under conditions to allow specific binding to the protein; and dissociating the antibodies from the protein, thereby obtaining purified antibodies. Such antibodies can include, but are not limited to, polyclonal, monoclonal, chimeric, and single chain antibodies, Fab fragments, and fragments produced by a Fab expression library. Neutralizing antibodies, such as those which inhibit dimer formation, are especially preferred for therapeutic use. Monoclonal antibodies to the protein can be prepared using any technique which provides for the production of antibody molecules by continuous cell lines in culture. These include, but are not limited to, the hybridoma, the human B-cell hybridoma, and the EBV-hybridoma techniques. In addition, techniques developed for the production of chimeric antibodies can be used. (See, e.g., Pound (1998) Immunochemical Protocols, Methods Mol Biol Vol. 80.) Alternatively, techniques described for the production of single chain antibodies can be employed. Fabs which contain specific binding sites for the protein can also be generated. Various immunoassays can be used to identify antibodies having the desired specificity. Numerous protocols for competitive binding or immunoradiometric assays using either polyclonal or monoclonal antibodies with established specificities are well known in the art.

Yet further, an agonist of the protein can be administered to a subject to treat a disorder associated with decreased expression, longevity or activity of the protein.

An additional aspect of the invention relates to the administration of a pharmaceutical or sterile composition, in conjunction with a pharmaceutically acceptable carrier, for any of the therapeutic applications discussed above. Such pharmaceutical compositions can consist of the protein or antibodies, mimetics, agonists, antagonists, or inhibitors of the protein. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a subject alone or in combination with other agents, drugs, or hormones.

The pharmaceutical compositions utilized in this invention can be administered by any number of routes including, but not limited to, oral, intravenous, intramuscular, intra-arterial, intramedullary, intrathecal, intraventricular, transdernal, subcutaneous, intraperitoneal, intranasal, enteral, topical, sublingual, or rectal means.

In addition to the active ingredients, these pharmaceutical compositions can contain pharmaceutically-acceptable carriers comprising excipients and auxiliaries which facilitate processing of the active compounds into preparations which can be used pharmaceutically. Further details on techniques for formulation and administration can be found in the latest edition of Remington's Pharmaceutical Sciences (Mack Publishing, Easton Pa.).

For any compound, the therapeutically effective dose can be estimated initially either in cell culture assays or in animal models such as mice, rats, rabbits, dogs, or pigs. An animal model can also be used to determine the concentration range and route of administration. Such information can then be used to determine useful doses and routes for administration in humans.

A therapeutically effective dose refers to that amount of active ingredient which ameliorates the symptoms or condition. Therapeutic efficacy and toxicity can be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population) statistics. Any of the therapeutic compositions described above can be applied to any subject in need of such therapy, including, but not limited to, mammals such as dogs, cats, cows, horses, rabbits, monkeys, and most preferably, humans.

Stem Cells and Their Use SEQ ID NOs:1-48 can be useful in the differentiation of stem cells. Eukaryotic stem cells are able to differentiate into the multiple cell types of various tissues and organs and to play roles in embryogenesis and adult tissue regeneration (Gearhart (1998) Science 282:1061-1062; Watt and Hogan (2000) Science 287:1427-1430). Depending on their source and developmental stage, stem cells can be totipotent with the potential to create every cell type in an organism and to generate a new organism, pluripotent with the potential to give rise to most cell types and tissues, but not a whole organism; or multipotent cells with the potential to differentiate into a limited number of cell types. Stem cells can be transfected with polynucleotides which can be transiently expressed or can be integrated within the cell as transgenes.

Embryonic stem (ES) cell lines are derived from the inner cell masses of human blastocysts and are pluripotent (Thomson et al. (1998) Science 282:1145-1147). They have normal karyotypes and express high levels of telomerase which prevent senescence and allow the cells to replicate indefinitely. ES cells produce derivatives that give rise to embryonic epidermal, mesodermal and endodermal cells. Embryonic germ (EG) cell lines, which are produced from primordial germ cells isolated from gonadal ridges and mesenteries, also show stem cell behavior (Shamblott et al. (1998) Proc Natl Acad Sci 95:13726-13731). EG cells have normal karyotypes and appear to be pluripotent.

Organ-specific adult stem cells differentiate into the cell types of the tissues from which they were isolated. They maintain their original tissues by replacing cells destroyed from disease or injury. Adult stem cells are multipotent and under proper stimulation can be used to generate cell types of various other tissues (Vogel (2000) Science 287:1418-1419). Hematopoietic stem cells from bone marrow provide not only blood and immune cells, but can also be induced to transdifferentiate to form brain, liver, heart, skeletal muscle and smooth muscle cells. Similarly mesenchymal stem cells can be used to produce bone marrow, cartilage, muscle cells, and some neuron-like cells, and stem cells from muscle have the ability to differentiate into muscle and blood cells (Jackson et al. (1999) Proc Natl Acad Sci 96:14482-14486). Neural stem cells, which produce neurons and glia, can also be induced to differentiate into heart, muscle, liver, intestine, and blood cells (Kuhn and Svendsen (1999) BioEssays 21:625-630); Clarke et al. (2000) Science 288:1660-1663; Gage (2000) Science 287:1433-1438; and Galli et al. (2000) Nature Neurosci 3:986-991).

Neural stem cells can be used to treat neurological disorders such as Alzheimer's disease, Parkinson's disease, and multiple sclerosis and to repair tissue damaged by strokes and spinal cord injuries. Hematopoietic stem cells can be used to restore immune function in immunodeficient patients or to treat autoimmune disorders by replacing autoreactive immune cells with normal cells to treat diseases such as multiple sclerosis, scleroderma, rheumatoid arthritis, and systemic lupus erythematosus. Mesenchymal stem cells can be used to repair tendons or to regenerate cartilage to treat arthritis. Liver stem cells can be used to repair liver damage. Pancreatic stem cells can be used to replace islet cells to treat diabetes. Muscle stem cells can be used to regenerate muscle to treat muscular dystrophies (Fontes and Thomson (1999) BMJ 319:1-3; Weissman (2000) Science 287:1442-1446 Marshall (2000) Science 287:1419-1421; and Marmont (2000) Ann Rev Med 51:115-134).

### EXAMPLES

It is to be understood that this invention is not limited to the particular devices, machines, materials and methods described. Although particular embodiments are described, equivalent embodiments can be used to practice the invention. The described embodiments are provided to illustrate the invention and are not intended to limit the scope of the invention which is limited only by the appended claims.

cDNA Library Construction

The cDNA library, LATRNOT01, was selected as an example to demonstrate library construction. The LATRNOT01 cDNA library was constructed from left atrial tissue obtained from a 51 -year-old Caucasian female who died of cerebral hemorrhage.

The frozen tissue was homogenized using a pestle and mortar and lysed using a POLYTRON homogenizer (Brinkmann Instruments, Westbury N.Y.) in guanidinium isothiocyanate solution. The lysate was centrifuged over a 5.7 M CsCl cushion using an SW28 swinging bucket rotor in an L8-70M ultracentrifuge (Beckman Coulter, Fullerton Calif.) for 18 hours at 25,000 rpm and ambient temperature. The RNA was extracted twice with phenol, pH 8.0, precipitated using 0.3 M sodium acetate and 2.5 volumes of ethanol, resuspended in RNAse-free water, and treated with DNAse at 37C. The mRNA was isolated using the OLIGOTEX kit (Qiagen, Chatsworth Calif.) and used to construct the cDNA library.

The mRNA was handled according to the recommended protocols in the SUPERSCRIPT plasmid system (Life Technologies, Gaithersburg Md.). cDNAs were fractionated on a SEPHAROSE CL4B column (Amersham Pharmacia Biotech (APB), Piscataway N.J.), and those cDNAs exceeding 400 bp were ligated into the XhoI and EcoRI sites of the λ UNIZAP vector (Stratagene, La Jolla Calif.). The vector which contained the PBLUESCRIPT phagemid was subsequently transformed into XL1-BLUEMRF host cells (Stratagene). The phagemid forms of individual cDNA clones were obtained by the in vivo excision process, in which the host bacterial strain was co-infected with both the λ library phage and an f1 helper phage. Enzymes derived from both the library-containing and helper phage nicked the λ DNA, initiated new DNA synthesis from defined sequences on the λ target DNA, and created a smaller, single stranded circular phagemid DNA molecule that included all DNA sequences of the PBLUESCRIPT phagemid and the cDNA insert. The phagemid DNA was secreted from the cells, purified, and used to re-infect fresh host cells, where the double stranded phagemid DNA was produced.

II Isolation and Sequencing of cDNA Clones

Plasmid DNA was released from the bacterial cells and purified using the REAL PREP 96 plasmid kit (Qiagen). This kit enabled the simultaneous purification of 96 samples in a 96-well block using multi-channel reagent dispensers. The recommended protocol was employed except for the following changes: 1) the bacteria were cultured in 1 ml of sterile TERRIFIC BROTH (BD Biosciences, San Jose Calif.) with carbenicillin at 25 mg/L and glycerol at 0.4%; 2) after inoculation, the cells were culture for 19 hours and then lysed in 0.3 ml of lysis buffer; and 3) the plasmid DNA pellet was precipitated in isopropanol and then resuspended in 0.1 ml of distilled water. After the last step in the protocol, samples were transferred to a 96-well block for storage at 4 C.

The cDNAs were prepared using a MICROLAB 2200 system (Hamilton, Reno Nev.) in combination with DNA ENGINE thermal cyclers (MJ Research, Watertown Mass.). The cDNAs were sequenced by the method of Sanger and Coulson (1975; J Mol Biol 94:441-448) using ABI PRISM 373, 377 or 3700 DNA sequencing systems (ABI). Most of the cDNAs were sequenced using standard ABI protocols and kits at solution volumes of 0.25x-1.0x. In the alternative, some of the cDNAs were sequenced using solutions and dyes from APB.

III Selection, Assembly, and Characterization of Sequences

The polynucleotides used for co-expression analysis were assembled from EST sequences, 5′ and 3′ long read sequences, and full length coding sequences. The assembly process is described as follows. EST sequence chromatograms were processed and verified. Quality scores were obtained using PHRED (Ewing et al. (1998) Genome Res 8:175-185; Ewing and Green (1998) Genome Res 8:186-194), and edited sequences were loaded into a relational database management system (RDBMS). The sequences were clustered using BLAST with a product score of 50. All clusters of two or more sequences created a bin which represents one transcribed gene.

Assembly of the component sequences within each bin was performed using a modification of Phrap, a publicly available program for assembling DNA fragments (Green, P. University of Washington, Seattle Wash.). Bins that showed 82% identity from a local pair-wise alignment between any of the consensus sequences were merged.

Bins were annotated by screening the consensus sequence in each bin against public databases, such as GBpri and GenPept from NCBI. The annotation process involved a FASTn screen against the GBpri database in GenBank. Those hits with a percent identity of greater than or equal to 75% and an alignment length of greater than or equal to 100 base pairs were recorded as homolog hits. The residual unannotated sequences were screened by FASTx against GenPept. Those hits with an E value of less than or equal to 10⁻⁸ were recorded as homolog hits.

Sequences were then reclustered using BLASTn and Cross-Match, a program for rapid amino acid and nucleic acid sequence comparison and database search (Green, supra), sequentially. Any BLAST alignment between a sequence and a consensus sequence with a score greater than 150 was realigned using cross-match. The sequence was added to the bin whose consensus sequence gave the highest Smith-Waterman score (Smith et al. (1992) Protein Engineering 5:35-51) amongst local alignments with at least 82% identity. Non-matching sequences were moved into new bins, and assembly processes were repeated.

IV Homology Searching of Polynucleotides and Their Encoded Proteins

The polynucleotides of the Sequence Listing or their encoded proteins were used to query databases such as GenBank, SwissProt, BLOCKS, and the like. These databases that contain previously identified and annotated sequences or domains were searched using BLAST or BLAST 2 (Altschul et al. supra; Altschul, supra) to produce alignments and to determine which sequences were exact matches or homologs. The alignments were to sequences of prokaryotic (bacterial) or eukaryotic (animal, fungal, or plant) origin. Alternatively, algorithms such as the one described in Smith and Smith (1992, Protein Engineering 5:35-51) could have been used to deal with primary sequence patterns and secondary structure gap penalties. All of the sequences disclosed in this application have lengths of at least 49 nucleotides, and no more than 12% uncalled bases (where N is recorded rather than A, C, G, or T).

As detailed in Karlin and Altschul (1993; Proc Natl Acad Sci 90:5873-5877), BLAST matches between a query sequence and a database sequence were evaluated statistically and only reported when they satisfied the threshold of 10⁻²⁵ for nucleotides and 10⁻¹⁴ for peptides. Homology was also evaluated by product score calculated as follows: the % nucleotide or amino acid identity [between the query and reference sequences] in BLAST is multiplied by the % maximum possible BLAST score [based on the lengths of query and reference sequences] and then divided by 100. In comparison with hybridization procedures used in the laboratory, the electronic stringency for an exact match was set at 70, and the conservative lower limit for an exact match was set at approximately 40 (with 1-2% error due to uncalled bases).

The BLAST software suite, freely available sequence comparison algorithms (NCBI, Bethesda Md.; http://www.ncbi.nlm.nih.gov/gorf/12.html), includes various sequence analysis programs including “blastn” that is used to align nucleic acid molecules and BLAST 2 that is used for direct pairwise comparison of either nucleic or amino acid molecules. BLAST programs are commonly used with gap and other parameters set to default settings, e.g.: Matrix: BLOSUM62; Reward for match: 1; Penalty for mismatch: -2; Open Gap: 5 and Extension Gap: 2 penalties; Gap x drop-off: 50; Expect: 10; Word Size: 11; and Filter: on. Identity or similarity is measured over the entire length of a sequence or some smaller portion thereof. Brenner et al. (1998; Proc Natl Acad Sci 95:6073-6078, incorporated herein by reference) analyzed the BLAST for its ability to identify structural homologs by sequence identity and found 30% identity is a reliable threshold for sequence alignments of at least 150 residues and 40%, for alignments of at least 70 residues.

The polynucleotides of this application were compared with assembled consensus sequences or templates found in the LIFESEQ GOLD database. Component sequences from cDNA, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to polynucleotide bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another and those having local similarity of at least 82% were combined and reassembled. Bins having templates with less than 95% local identity were split. Templates were subjected to analysis by STITCHER/EXON MAPPER algorithms that analyze the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of <1×10⁻⁸. The templates were also subjected to frameshift FASTx against GENPEPT, and homolog match was defined as having an E-value of <1×10⁻⁸. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. Nos. 08/812,290 and 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis Mo.; http://pfam.wustl.edu/).

The polynucleotide was further analyzed using MACDNASIS PRO software (Hitachi Software Engineering), and LASERGENE software (DNASTAR) and queried against public databases such as the GenBank rodent, mammalian, vertebrate, prokaryote, and eukaryote databases, SwissProt, BLOCKS, PRINTS, PFAM, and Prosite.

V Description of Known Cardiac Muscle-Associated Genes

Twelve known cardiac muscle-associated genes were selected to identify novel polynucleotides that are closely associated with cardiac muscle function. These known genes were atrial regulatory myosin, ventricular myosin alkali light chain, cardiac troponin, cardiac ventricular myosin, cardiodilatin, creatine kinase M, myoglobin, natriuretic peptide precursor, sarcomeric mitochondrial creatine kinase, telethonin, titin, and urocortin.

Brief descriptions of the known cardiac muscle-associated genes and their expression in cardiac disorders are presented below.

| GENE | DESCRIPTION AND REFERENCES |
|---|---|
| atrial regulatory | Predominant regulatory myosin light chain |
| myosin | isoform in adult atrial muscle. Differentially |
| | expressed in cardiovascular development and |
| | disease. Fewell et al. (1998) J Clin Invest |
| | 101:2630-2639; Hailstones et al. (1992) J. Biol. |
| | Chem. 267:23295-23300. |
| ventricular myosin | Muscle fiber protein. Differentially expressed in |
| alkali light chain | altered cardiovascular function and in myocardial |
| | hypertrophy. Morano et al. (1997) J Mol Cell |
| | Cardiol 29:1177-1187. |
| troponin | Marker of cardiac injury. Feng et al. (1998) Am |
| | J Clin Pathol 110:70-77; Luscher et al. (1998) |
| | Cardiology 89:222-228; and Kost et al. (1998) |
| | Arch Pathol Lab Med 122:245-251. |
| cardiac ventricular | Muscle fiber protein. Expressed in cardiac |
| myosin | remodeling after myocardial infarction. |
| | Differentially expressed in altered cardiovascular |
| | function. Trahair et al. (1993) J Mol Cell |
| | Cardiol 25:577-585. |
| cardiodilatin | Differentially expressed following myocardial |
| | infarction. Induces vasorelaxation. Gidh-Jain |
| | et al. (1998) J Mol Cell Cardiol 30:627-637; |
| | Magga et al. (1998) Ann Med 30(S1):39-45. |
| creatine kinase M | Marker of cardiac injury. Feng, supra; Luscher, |
| | supra; and Kost, supra. |
| myoglobin | Marker of cardiac injury. Feng, supra; Luscher, |
| | supra; and Kost, supra. |
| natriuretic peptide | See cardiodilatin. |
| precursor | |
| sarcomeric | Essential enzyme in energy metabolism, |
| mitochondrial creatine | particularly in tissue with high energy |
| kinase | requirements. Klein et al. (1991) J Biol Chem |
| | 266:18058-18065; Qin et al. (1997) J Biol |
| | Chem 272:25210-25216. |
| telethonin | Sarcomeric protein of heart and skeletal muscle. |
| | Valle et al. (1997) FEBS Lett. 415:163-168; |
| | Mayans et al. (1998) Nature 395:863-869. |
| titin | Muscle fiber protein. Temporal and spatial control |
| | of sarcomere assembly. Differentially expressed |
| | after atrial fibrillation. Ausma et al. (1997) Am |
| | J Pathol 151:985-997; Mayans, supra. |
| urocortin | Stimulates atrial natriuretic peptide secretion. |
| | Expression increased following cardiac injury. |
| | Protects cardiac myocytes from hypoxic death. |
| | Ikeda et al. (1998) Biochem. Biophys Res |
| | Commun 250:298-304; Asaba et al. (1998) Brain |
| | Res 806:95-103; and Okosi et al. (1998) |
| | Neuropeptides 32:167-171. |

VI Co-Expression Among Known Marker Genes and Novel Polynucleotides

GBA identified 48 novel polynucleotides from a total of 45,233 assembled sequences that showed strong expression and association with the known cardiac muscle-associated genes. The process was reiterated until the number of polynucleotides was reduced to the final 48 polynucleotides shown below. Each of the 48 polynucleotides is co-expressed with at least one of the twelve known genes with a p-value of less than 10⁻⁰⁵.

The co-expression of the novel polynucleotides and the known genes are shown in Table 1-1, 1-2, and 1-3. The novel polynucleotides are listed along the top of the table by their SEQ ID NO, and the known genes, by their names in the rows down the side of the table. The entries in the table are the negative log of the p-value (−log p) for the co-expression of two sequences. For each polynucleotide, the p-value is the probability that the observed co-expression is due to chance, using the Fisher Exact Test.

The highest co-expression value is obtained when the highest p-value found in a vertical column below the SEQ ID NO (clone number) is correlated with the name of a known marker gene listed for that row. For example, SEQ ID NO:4, has a p-value of 19 as it co-expresses with cardiac ventricular myosin. This highly significant p-value substantiates that SEQ ID NO:4, SEQ ID NO:49, and an antibody which specifically binds SEQ ID NO:49 can be used as surrogate markers for cardiac ventricular myosin in a diagnostic assay for myocardial infarction.

The data above can be summarized by reducing it to a single highest co-expression (−log p) value for each intersecting known gene and unknown polynucleotide and naming at least one disorder associated with expression of the known gene. A summary table is shown below:

| SEQ | | | |
|---|---|---|---|
| ID | p- | | |
| NO | value | Gene | Disorder |
| 1 | 7 | atrial regulatory myosin | cardiac injury |
| 2 | 6 | natriuretic peptide precursor | myocardial infarction |
| 3 | 7 | telethonin | atrial fibrillation |
| 4 | 19 | cardiac ventricular myosin | myocardial infarction |
| 5 | 9 | creatine kinase M | cardiac injury |
| 6 | 11 | titin | atrial fibrillation |
| 7 | 10 | troponin | cardiac injury |
| 8 | 6 | natriuretic peptide precursor | myocardial infarction |
| 9 | 6 | urocortin | myocardial infarction |
| 10 | 12 | telethonin | atrial fibrillation |
| 11 | 8 | creatine kinase M | cardiac injury |
| 12 | 9 | atrial regulatory myosin | cardiac injury |
| 13 | 22 | titin | atrial fibrillation |
| 14 | 8 | ventricular myosin alkali light | myocardial hypertrophy |
| | | chain | |
| 15 | 10 | titin | atrial fibrillation |
| 16 | 7 | titin | atrial fibrillation |
| 17 | 8 | telethonin | atrial fibrillation |
| 18 | 6 | urocortin | myocardial infarction |
| 19 | 11 | creatine kinase M | cardiac injury |
| 20 | 13 | myoglobin | cardiac injury |
| 21 | 10 | ventricular myosin alkali light | myocardial hypertrophy |
| | | chain | |
| 22 | 10 | troponin | cardiac injury |
| 23 | 11 | titin | atrial fibrillation |
| 24 | 7 | ventricular myosin alkali light | myocardial hypertrophy |
| | | chain | |
| 25 | 9 | ventricular myosin alkali light | myocardial hypertrophy |
| | | chain | |
| 26 | 18 | creatine kinase M | cardiac injury |
| 27 | 19 | ventricular myosin alkali light | myocardial hypertrophy |
| | | chain | |
| 28 | 21 | creatine kinase M | cardiac injury |
| 29 | 5 | sarcomeric mitoch. creatine | hypertension |
| | | kinase | |
| 30 | 15 | myoglobin | cardiac injury |
| 31 | 7 | telethonin | atrial fibrillation |
| 32 | 8 | creatine kinase M | cardiac injury |
| 33 | 11 | titin | atrial fibrillation |
| 34 | 9 | atrial regulatory myosin | cardiac injury |
| 35 | 8 | creatine kinase M | cardiac injury |
| 36 | 7 | cardiac ventricular myosin | myocardial infarction |
| 37 | 16 | myoglobin | cardiac injury |
| 38 | 11 | myoglobin | cardiac injury |
| 39 | 21 | creatine kinase M | cardiac injury |
| 40 | 11 | creatine kinase M | cardiac injury |
| 41 | 20 | creatine kinase M | cardiac injury |
| 42 | 8 | titin | atrial fibrillation |
| 43 | 6 | cardiac ventricular myosin | myocardial infarction |
| 44 | 7 | cardiodilantin | myocardial infarction |
| 45 | 10 | telethonin | atrial fibrillation |
| 46 | 11 | creatine kinase M | cardiac injury |
| 47 | 9 | atrial regulatory myosin | cardiac injury |
| 48 | 9 | telethonin | atrial fibrillation |

* p-value (- log p) = 5 is highly significant

VII Description of the Polynucleotides Identified Using GBA

Using the method of Walker (supra), 48 polynucleotides that exhibit strong association, or co-expression, with cardiac muscle-associated genes have been identified.

Polynucleotides comprising the nucleic acid sequences of SEQ ID NOs:1-48 of the present invention fied as Incyte Clones 2045674, 188552, 465676, 3601719, 305781, 971441, 3445829, 189299, 2396760, 919893, 2837330, 1737459, 058201, 767447, 5449893, 2951269, 282977, 3178454, 3563859, 985730, 3684987, 986166, 1887508, 1006416, 975169, 4152861, 986464, 118472, 1314633, 1997439, 2638878, 3795510, 1413537, 1623157, 3009303, 3434460, 5022769, 944140, 3445829, 3016490, 4151935, 3719652, 3046106, 3012947, 466761, 1644171, 3009806, and 5578191, respectively; and assembled according to Example III. As described in Example IV, BLAST and other motif searches were performed for each sequence. SEQ ID NOs:1-48 were translated, and identity with known sequences was sought. Proteins comprising SEQ ID NOs:49-62 were also analyzed using BLAST and other motif search tools as disclosed in Example VI. The details of the various analyses are described in Table 2.

VIII Hybridization Technologies and Analyses

Immobilization of Polynucleotides on a Substrate

The polynucleotides are applied to a substrate by one of the following methods. A mixture of polynucleotides is fractionated by gel electrophoresis and transferred to a nylon membrane by capillary transfer. Alternatively, the polynucleotides are individually ligated to a vector and inserted into bacterial host cells to form a library. The polynucleotides are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37 C. for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris-HCl, pH 8.0), and twice in 2×SSC for 10 min each. The membrane is then UV irradiated in a STRATALINKER UV-crosslinker (Stratagene).

In the second method, polynucleotides are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning, Acton Mass.) by ultrasound in 0.1% SDS and acetone, etching in 4% hydrofluoric acid (VWR Scientific Products, West Chester Pa.), coating with 0.05% aminopropyl silane (Sigma-Aldrich) in 95% ethanol, and curing in a 110 C. oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford Mass.) for 30 min at 60 C.; then the arrays are washed in 0.2% SDS and rinsed in distilled water as before.

Probe Preparation for Membrane Hybridization

Hybridization probes derived from the polynucleotides of the Sequence Listing are employed for screening cDNAs, mRNAs, or genomic DNA in membrane-based hybridizations. Probes are prepared by diluting the polynucleotides to a concentration of 40-50 ng in 45 μl TE buffer, denaturing by heating to 100 C. for five min, and briefly centrifuging. The denatured polynucleotide is then added to a REDIPRIME tube (APB), gently mixed until blue color is evenly distributed, and briefly centrifuged. Five μl of [³²P]dCTP is added to the tube, and the contents are incubated at 37 C. for 10 min. The labeling reaction is stopped by adding 5 μl of 0.2M EDTA, and probe is purified from unincorporated nucleotides using a PROBEQUANT G-50 microcolumn (APB). The purified probe is heated to 100 C. for five min, snap cooled for two min on ice, and used in membrane-based hybridizations as described below.

Probe Preparation for Polymer Coated Slide Hybridization

Hybridization probes derived from mRNA isolated from samples are employed for screening polynucleotides of the Sequence Listing in array-based hybridizations. Probe is prepared using the GEMbright kit (Incyte Genomics) by diluting mRNA to a concentration of 200 ng in 9 μl TE buffer and adding 5 μl 5×buffer, 1 μl 0.1 M DTT, 3 μl Cy3 or Cy5 labeling mix, 1 μl RNAse inhibitor, 1 μl reverse transcriptase, and 5 μl 1×yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37 C. for two hr. The reaction mixture is then incubated for 20 min at 85 C., and probes are purified using two successive CHROMA SPIN+TE 30 columns (Clontech, Palo Alto Calif.). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800×g, and the pellet is resuspended in 12 μl resuspension buffer, heated to 65 C. for five min, and mixed thoroughly. The probe is heated and mixed as before and then stored on ice. Probe is used in high density array-based hybridizations as described below.

Membrane-based Hybridization

Membranes are pre-hybridized in hybridization solution containing 1% Sarkosyl and 1×high phosphate buffer (0.5 M NaCl, 0.1 M Na₂HPO₄, 5 mM EDTA, pH 7) at 55 C. for two hr. The probe, diluted in 15 ml fresh hybridization solution, is then added to the membrane. The membrane is hybridized with the probe at 55 C. for 16 hr. Following hybridization, the membrane is washed for 15 min at 25 C. in 1 mM Tris (pH 8.0), 1% Sarkosyl, and four times for 15 min each at 25 C. in 1 mM Tris (pH 8.0). To detect hybridization complexes, XOMAT-AR film (Eastman Kodak, Rochester N.Y.) is exposed to the membrane overnight at −70 C., developed, and examined visually.

Polymer Coated Slide-based Hybridization

Probe is heated to 65 C. for five min, centrifuged five min at 9400 rpm in a 5415 C. microcentrifuge (Eppendorf Scientific, Westbury N.Y.), and then 18 μl are aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60 C. The arrays are washed for 10 min at 45 C. in 1×SSC, 0.1% SDS, and three times for 10 min each at 45 C. in 0.1×SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of genes in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to equal numbers of probes derived from both biological samples give a distinct combined fluorescence (Shalon WO95/35505).

Hybridization complexes are detected with a microscope equipped with an INNOVA 70 mixed gas 10 W laser (Coherent, Santa Clara Calif.) capable of generating spectral lines at 488 nm for excitation of Cy3 and at 632 nm for excitation of Cy5. The excitation laser light is focused on the array using a 20× microscope objective (Nikon, Melville N.Y.). The slide containing the array is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

IX Transcript Imaging

The transcript image performed using the LIFESEQ GOLD database (Aug00rel, Incyte Genomics) allowed assessment of the relative abundance of expressed polynucleotides in one or more cDNA libraries. Criteria for transcript imaging include category, number of cDNAs per library, description of the library, and the like

All sequences and cDNA libraries in the LIFESEQ database were categorized by system, organ/tissue and cell type. The categories included cardiovascular system, connective tissue, digestive system, embryonic structures, endocrine system, exocrine glands, female and male reproductive, germ cells, hemic/immune system, liver, musculoskeletal system, nervous system, pancreas, respiratory system, sense organs, skin, stomatognathic system, unclassified/mixed, and the urinary tract. For each category, the number of libraries in which the sequence was expressed were counted and shown over the total number of libraries in that category. In some transcript images, all normalized or pooled libraries, which have high copy number sequences removed prior to processing, and all mixed or pooled tissues, which are considered non-specific in that they contain more than one tissue type or more than one subject's tissue, can be excluded from the analysis. Cell lines and/or fetal tissue data can also be disregarded unless the elucidation of inherited disorders would be furthered by their inclusion in the analysis.

For diagnostic purposes, the standards to which biopsied samples would be compared are: cytologically normal, non-diseased samples versus samples which had been diagnosed with specific cardiac disorders including, but not limited to, atherosclerosis, arteriosclerosis, atrial fibrillation, cancer (myxoma) and complications of cancer, cardiac injury, congestive heart failure, coronary artery disease, hypertension, hypertrophic cardiomyopathy, myocardial hypertrophy, myocardial infarction, and plaque.

For purposes of example, the transcript images for SEQ ID NOs:29 and 44 are shown below. The first column shows library name; the second column, the number of cDNAs sequenced in that library; the third column, the description of the library; and the fourth column, absolute abundance of the transcript in the library.

SEQ ID NO:29 (Category: Cardiovascular*)

| | | | Abun- | % Abun- |
|---|---|---|---|---|
| Library | cDNA | Description | dance | dance |
| HEARNOT06 | 3685 | heart, hypertension, 44M | 2 | 0.0543 |
| HEARFET05 | 2524 | heart, fetal, M | 1 | 0.0396 |
| HEARFET02 | 6919 | heart, hypoplastic | 1 | 0.0145 |
| | | left, fetal, 23wM | | |

*No libraries were removed from the analysis.

SEQ ID NO:44 (Category: Cardiovascular*)

| | | | Abun- | % Abun- |
|---|---|---|---|---|
| Library | cDNA | Description | dance | dance |
| HEALDIT02 | 4171 | left ventricle, mw/ | 1 | 0.0240 |
| | | myocardial infarction, | | |
| | | 56M | | |
| HEARFET02 | 6919 | heart, hypoplastic left, | 1 | 0.0145 |
| | | fetal, 23wM | | |

*Normalized and pooled libraries were removed from the analysis.

SEQ ID NOs:29 and 44 were differentially expressed when compared by percent abundance to useful standards (i.e., the up-regulation of SEQ ID NOs:29 in heart tissue of a deceased victim who was shot to death is not a comparison that would be made in a diagnostic setting). More importantly, these sequences are not differentially expressed in any normal tissue or diagnostic of any other cardiac disorder.

The differential expression of SEQ ID NOs:29, and 44, respectively, in tissue associated with hypertension and myocardial infarction, respectively, supports the use of the sequences as a surrogate markers for sarcomeric mitochondrial creatine kinase and cardiodilantin, respectively. These transcript images verify GBA analysis (see Example VI above).

X Complementary Molecules

The complement of the novel polynucleotide, from about 5 bp (e.g., a PNA) to about 5000 bp (e.g., the complement of a cDNA insert), are used to detect or inhibit gene expression. These molecules are selected using LASERGENE software (DNASTAR). Detection is described in Example VIII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in “triple helix” base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if appropriate elements for inducing vector replication are used in the transformation/expression system.

Stable transformation of appropriate dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the polynucleotide encoding the protein.

XI Protein Expression

Expression and purification of the protein are achieved using either a cell expression system or an insect cell expression system. The pUB6/V5-His vector system (Invitrogen, Carlsbad Calif.) is used to express protein in CHO cells. The vector contains the selectable bsd gene, multiple cloning sites, the promoter/enhancer sequence from the human ubiquitin C gene, a C-terminal V5 epitope for antibody detection with anti-V5 antibodies, and a C-terminal polyhistidine (6×His) sequence for rapid purification on PROBOND resin (Invitrogen). Transformed cells are selected on media containing blasticidin.

Spodoptera frugiperda (Sf9) insect cells are infected with recombinant Autographica californica nuclear polyhedrosis virus (baculovirus). The polyhedrin gene is replaced with the polynucleotide by homologous recombination and the polyhedrin promoter drives transcription. The protein is synthesized as a fusion protein with 6×his which enables purification as described above. Purified protein is used in the following activity and to make antibodies.

XII Production of Antibodies

The protein is purified using polyacrylamide gel electrophoresis and used to immunize mice or rabbits. Antibodies are produced using the protocols below. Alternatively, the amino acid sequence of the expressed protein is analyzed using LASERGENE software (DNASTAR) to determine regions of high antigenicity. An antigenic epitope, usually found near the C-terminus or in a hydrophilic region is selected, synthesized, and used to raise antibodies. Typically, epitopes of about 15 residues in length are produced using an ABI 431A peptide synthesizer (ABI) using FMOC-chemistry and coupled to KLH (Sigma-Aldrich) by reaction with N-maleimidobenzoyl-N-hydroxysuccinimide ester to increase antigenicity.

Rabbits are immunized with the epitope-KLH complex in complete Freund's adjuvant. Immunizations are repeated at intervals thereafter in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washing, and reacting with radio-iodinated goat anti-rabbit IgG. Methods well known in the art are used to determine antibody titer and the amount of complex formation.

XIII Purification of Naturally Occuring Protein Using Specific Antibodies

Naturally occurring or recombinant protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIV Screening Molecules for Specific Binding Using Polynucleotide or Protein

The polynucleotide, or fragments thereof, or the protein, or portions thereof, are labeled with ³²P-dCTP, Cy3-dCTP, or Cy5-dCTP (APB), or with BIODIPY or FITC (Molecular Probes, Eugene Oreg.), respectively. Libraries of candidate molecules or compounds previously arranged on a substrate are incubated in the presence of composition, a labeled polynucleotide or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XV Two-Hybrid Screen

A yeast two-hybrid system, MATCHMAKER LexA Two-Hybrid system (Clontech Laboratories, Palo Alto Calif.), is used to screen for peptides that bind the protein of the invention. A polynucleotide encoding the protein is inserted into the multiple cloning site of a pLexA vector, ligated, and transformed into E. coli. cDNA, prepared from mRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48 [p8op-lacZ] cells using a polyethylene glycol/lithium acetate protocol. Transformed yeast cells are plated on synthetic dropout (SD) media lacking histidine (-His), tryptophan (-Trp), and uracil (-Ura), and incubated at 30 C. until the colonies have grown up and are counted. The colonies are pooled in a minimal volume of 1×TE (pH 7.5), replated on SD/-His/-Leu/-Trp/-Ura media supplemented with 2% galactose (Gal), 1% raffinose (Raf), and 80 mg/ml 5-bromo-4-chloro-3-indolyl β-d-galactopyranoside (X-Gal), and subsequently examined for growth of blue colonies. Interaction between expressed protein and cDNA fusion proteins activates expression of a LEU2 reporter gene in EGY48 and produces colony growth on media lacking leucine (-Leu). Interaction also activates expression of β-galactosidase from the p8op-lacZ reporter construct that produces blue color in colonies grown on X-Gal.

Positive interactions between expressed protein and cDNA fusion proteins are verified by isolating individual positive colonies and growing them in SD/-Trp/-Ura liquid medium for 1 to 2 days at 30 C. A sample of the culture is plated on SD/-Trp/-Ura media and incubated at 30 C. until colonies appear. The sample is replica-plated on SD/-Trp/-Ura and SD/-His/-Trp/-Ura plates. Colonies that grow on SD containing histidine but not on media lacking histidine have lost the pLexA plasmid. Histidine-requiring colonies are grown on SD/Gal/Raf/X-Gall-Trp/-Ura, and white colonies are isolated and propagated. The pB42AD-cDNA plasmid, which contains a polynucleotide encoding a protein that physically interacts with the protein, is isolated from the yeast cells and characterized.

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

TABLE 1-1

| GENE NAME\SEQ ID NO* | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|
| atrial regulatory myosin | 7 | 5 | 3 | 13 | 2 | 2 | 10 | 5 | 1 | 7 | 5 | 9 | 7 | 3 | 1 | 2 |
| ventricular myosin alkali light chain | 5 | 4 | 4 | 18 | 8 | 9 | 9 | 4 | 2 | 11 | 6 | 6 | 14 | 8 | 5 | 4 |
| troponin | 6 | 5 | 5 | 10 | 3 | 1 | 10 | 5 | 1 | 8 | 7 | 8 | 6 | 2 | 1 | 0 |
| cardiac ventricular myosin | 4 | 4 | 3 | 19 | 6 | 9 | 7 | 4 | 2 | 8 | 7 | 5 | 17 | 5 | 7 | 5 |
| cardiodilatin | 4 | 3 | 4 | 10 | 2 | 1 | 5 | 3 | 1 | 4 | 5 | 7 | 4 | 1 | 1 | 0 |
| creatine kinase M | 6 | 4 | 6 | 16 | 9 | 9 | 7 | 4 | 2 | 10 | 8 | 6 | 21 | 6 | 8 | 5 |
| myoglobin | 4 | 4 | 6 | 17 | 8 | 10 | 7 | 4 | 2 | 9 | 5 | 8 | 19 | 3 | 9 | 3 |
| natriuretic peptide precursor | 6 | 6 | 2 | 9 | 0 | 1 | 5 | 6 | 1 | 5 | 2 | 6 | 4 | 1 | 2 | 1 |
| sarcomeric mitoch. creatine kinase | 7 | 4 | 7 | 16 | 7 | 5 | 8 | 4 | 2 | 11 | 6 | 6 | 12 | 3 | 5 | 2 |
| telethonin | 4 | 4 | 7 | 15 | 6 | 8 | 8 | 4 | 2 | 12 | 6 | 5 | 18 | 6 | 7 | 6 |
| titin | 4 | 4 | 6 | 18 | 9 | 11 | 5 | 4 | 2 | 11 | 8 | 5 | 22 | 5 | 10 | 7 |
| urocortin | 2 | 1 | 1 | 7 | 2 | 5 | 3 | 1 | 6 | 5 | 2 | 2 | 5 | 2 | 6 | 6 |

*entries in the table are the negative log of the p-value; an entry of 5 or greater is highly significant.

TABLE 1-2

| GENE NAME\SEQ ID NO* | 17 | 18 | 19 | 20 | 21 | 22 | 23 | 24 | 25 | 26 | 27 | 28 | 29 | 30 | 31 | 32 |
|---|---|---|---|---|---|---|---|---|---|---|---|---|---|---|---|---|
| atrial regulatory myosin | 2 | 2 | 4 | 10 | 7 | 8 | 1 | 6 | 6 | 11 | 15 | 7 | 2 | 12 | 1 | 2 |
| ventricular myosin alkali light chain | 6 | 1 | 10 | 8 | 10 | 6 | 5 | 7 | 9 | 15 | 19 | 17 | 2 | 11 | 5 | 4 |
| troponin | 2 | 0 | 4 | 5 | 5 | 10 | 0 | 6 | 5 | 9 | 14 | 7 | 3 | 9 | 0 | 0 |
| cardiac ventricular myosin | 7 | 2 | 9 | 9 | 8 | 5 | 6 | 5 | 7 | 14 | 16 | 18 | 4 | 10 | 6 | 7 |
| cardiodilatin | 1 | 0 | 2 | 7 | 5 | 5 | 1 | 4 | 3 | 6 | 8 | 5 | 1 | 9 | 0 | 1 |
| creatine kinase M | 7 | 0 | 11 | 9 | 7 | 7 | 7 | 7 | 7 | 18 | 17 | 21 | 4 | 14 | 4 | 8 |
| myoglobin | 7 | 2 | 9 | 13 | 8 | 7 | 10 | 5 | 7 | 14 | 16 | 20 | 3 | 15 | 6 | 6 |
| natriuretic peptide precursor | 3 | 1 | 4 | 5 | 9 | 3 | 1 | 2 | 5 | 6 | 12 | 5 | 1 | 10 | 1 | 2 |
| sarcomeric mitoch. creatine kinase | 6 | 0 | 10 | 9 | 7 | 8 | 5 | 5 | 6 | 14 | 13 | 15 | 5 | 13 | 5 | 6 |
| telethonin | 8 | 1 | 9 | 9 | 7 | 8 | 9 | 3 | 8 | 14 | 16 | 19 | 1 | 14 | 7 | 7 |
| titin | 5 | 2 | 10 | 12 | 9 | 7 | 11 | 6 | 5 | 16 | 15 | 18 | 4 | 14 | 6 | 7 |
| urocortin | 3 | 6 | 5 | 4 | 4 | 3 | 4 | 1 | 3 | 6 | 6 | 3 | 2 | 8 | 6 | 4 |

*entries in the table are the negative log of the p-value; an entry of 5 or greater is highly significant.

TABLE 1-3

| GENE NAME\SEQ ID NO* | 33 | 34 | 35 | 36 | 37 | 38 | 39 | 40 | 41 | 42 | 43 | 44 | 45 | 46 | 47 | 48 |
|---|---|---|---|---|---|---|---|---|---|---|---|---|---|---|---|---|
| atrial regulatory myosin | 8 | 9 | 1 | 5 | 10 | 11 | 9 | 3 | 11 | 4 | 3 | 2 | 5 | 7 | 9 | 3 |
| ventricular myosin alkali light chain | 7 | 7 | 6 | 5 | 14 | 8 | 13 | 11 | 18 | 5 | 4 | 3 | 8 | 10 | 9 | 9 |
| troponin | 6 | 8 | 3 | 4 | 10 | 10 | 10 | 4 | 10 | 4 | 5 | 3 | 3 | 8 | 5 | 2 |
| cardiac ventricular myosin | 6 | 7 | 8 | 7 | 14 | 7 | 16 | 10 | 15 | 6 | 6 | 4 | 6 | 11 | 8 | 7 |
| cardiodilatin | 4 | 4 | 2 | 1 | 6 | 10 | 5 | 2 | 8 | 6 | 5 | 7 | 3 | 5 | 2 | 2 |
| creatine kinase M | 8 | 7 | 8 | 4 | 13 | 8 | 21 | 11 | 20 | 7 | 3 | 4 | 7 | 11 | 7 | 6 |
| myoglobin | 8 | 7 | 5 | 4 | 16 | 11 | 20 | 9 | 19 | 6 | 5 | 6 | 8 | 9 | 8 | 7 |
| natriuretic peptide precursor | 5 | 4 | 1 | 1 | 4 | 6 | 8 | 2 | 7 | 2 | 1 | 2 | 4 | 5 | 3 | 4 |
| sarcomeric mitoch. creatine kinase | 9 | 5 | 7 | 3 | 13 | 8 | 19 | 7 | 17 | 5 | 4 | 4 | 7 | 9 | 8 | 5 |
| telethonin | 10 | 7 | 6 | 4 | 9 | 6 | 20 | 10 | 19 | 4 | 4 | 2 | 10 | 8 | 7 | 9 |
| titin | 11 | 7 | 8 | 5 | 11 | 7 | 17 | 9 | 19 | 8 | 3 | 4 | 9 | 11 | 8 | 6 |
| urocortin | 2 | 4 | 3 | 3 | 9 | 3 | 7 | 3 | 7 | 1 | 1 | 2 | 4 | 3 | 7 | 6 |

*entries in the table are the negative log of the p-value; an entry of 5 or greater is highly significant.

TABLE 2

| | | | Potential | | | |
|---|---|---|---|---|---|---|
| SEQ ID | Amino Acid | Potential Phosphorylation | glycosylation | | | Analytical |
| NO: | Residues | Sites | sites | Signature Sequence | Identification | Methods |
| 49 | 70 | S46 | | | | Motif |
| 50 | 552 | S541 S11 S15 S26 S54 S99 S108 | N148 N174 | K402 to T456 | Tropomodulin | Motif, BLAST |
| | | T118 S125 S134 S168 T197 T250 | N177 N223 | Synapsins | synapsin | BLOCKS |
| | | S312 S502 S520 T56 S77 T143 | N325 | | | |
| | | T281 S392 S400 T409 S435 S499 | | | | |
| | | S511 S533 | | | | |
| 51 | 260 | S35 S51 T124 S171 S183 Y154 | | | | Motif |
| 52 | 364 | T103 T125 T247 T274 S329 S5 | N4 | M1 to G49 Signal | Receptor | Motif, SigPept |
| | | S162 S242 S282 | | Peptide | glycosyl | PRINTS, BLOCKS |
| | | | | m42 TO c64 and D76 | hydrolase | |
| | | | | to C88 receptor | | |
| | | | | signatures | | |
| | | | | F173 to F182 | | |
| | | | | Glycosyl hydrolases | | |
| | | | | signature | | |
| 53 | 527 | S168 S232 S239 T314 S315 T332 | | | | Motif |
| | | T344 T373 T496 T512 S524 | | | | |
| 54 | 82 | T63 T67 | N29 | | | Motif |
| 56 | 193 | S4 S6 T60 S86 S148 T157 T60 | N2 | L86 to Y122 | HET-C, | Motif, BLAST |
| | | T126 | | Phosphatase | glycolipid | BLOCKS_DOMO |
| | | | | transforming 61K | transfer protein | |
| | | | | PDF1 | | |
| 57 | 174 | T49 S40 T72 S81 S21 S57 S141 | N19 | L8 to L29leucine | CNN, mitosin, | Motif, BLAST |
| | | | | zipper pattern | tropomyosin | BLOCKS, PRINTS |
| | | | | Y27 to E42 and E103 | | |
| | | | | to L118 secretin | | |
| | | | | receptor | | |
| | | | | E54 to K71 and E103 | | |
| | | | | to E131 tropomysin | | |
| | | | | receptor | | |
| | | | | Q95 to T148 | | |
| | | | | tropomysin | | |
| 58 | 230 | S27 T33 S58 T75 T209 | | S23 | Glycosyl | Motif, BLOCKS |
| | | | | Glycosaminoglycan | Transferase | |
| | | | | attachement site | | |
| | | | | P84 TO p95 | | |
| | | | | Aminoacyl tRNA | | |
| | | | | synthetase class-1 | | |
| | | | | signature | | |
| | | | | V119 to H129 | | |
| | | | | glycosyl | | |
| | | | | transferase | | |
| | | | | signature | | |
| 59 | 915 | T775 T56 S58 S74 T100 S140 | N426 N633 | L530 to S641 and | Ring finger | Motif, BLAST |
| | | S224 T240 S241 S291 T292 S308 | | P650 to S734 fn | protein, | PRINTS, BLOCKS, |
| | | S314 T320 S353 S367 T375 S382 | | family, L607 to | zincfinger | Pfam |
| | | S414 T422 S428 S455 T480 T502 | | Y625 and Y718 to | protein RFP | |
| | | S503 S513 S529 T608 T674 S767 | | E732 fibronectin | fibronectin | |
| | | T796 T20 T179 S329 T343 T361 | | V627 to G636 and | | |
| | | T369 S406 S538 S641 T668 S740 | | F720 to G729 | | |
| | | T849 S911 Y119 Y360 | | receptor | | |
| | | | | glycoprotein | | |
| | | | | signature | | |
| 60 | 163 | S125 S94 | | F74 to A93 smooth | Smooth muscle | Motif, |
| | | | | muscle protein 22 | protein, | BLOCKS_DOMO |
| | | | | G83 to S94 | proteoglycan | PRINTS |
| | | | | proteoglycan C- | | |
| | | | | terminal | | |
| 62 | 329 | S68 T67 T284 S318 | N316 | R28 “RGD” cell | Cardiac ankyrin | Motif, BLAST, |
| | | | | attachment sequence | repeat protein | PRINTS, BLOCKS, |
| | | | | L154 to L169, M187 | | Pfam |
| | | | | to L202, L220 to | | |
| | | | | F235, G249 to R258, | | |
| | | | | and L253 to L268 | | |
| | | | | ankyrin repeats | | |

What is claimed is:

1. A composition comprising a plurality of polynucleotides having the nucleic acid sequences of SEQ ID NOs:1-48 or the complements thereof.

2. An isolated polynucleotide comprising a nucleic acid sequence selected from SEQ ID NOs:1-48 and the complements thereof.

3. A composition comprising a polynucleotide of claim 2 and a labeling moiety.

4. A method of using a polynucleotide to screen a plurality of molecules to identify at least one ligand which specifically binds the polynucleotide, the method comprising:
a) combining the composition of claim 1 with a plurality of molecules under conditions to allow specific binding; and
b) detecting specific binding, thereby identifying a ligand which specifically binds a polynucleotide.

5. The method of claim 4 wherein the composition is attached to a substrate.

6. The method of claim 4 wherein the molecules to be screened are selected from DNA molecules, RNA molecules, peptide nucleic acids, mimetics, and proteins.

7. A method of using a polynucleotide to purify a ligand, the method comprising:
a) combining the polynucleotide of claim 2 with a sample under conditions to allow specific binding;
b) recovering the bound polynucleotide; and
c) separating the ligand from the bound polynucleotide, thereby obtaining purified ligand.

8. The method of claim 7 wherein the polynucleotide is attached to a substrate.

9. A method for using a polynucleotide to detect gene expression in a sample, the method comprising:
a) hybridizing the composition of claim 1 to a sample thereby forming at least one hybridization complex;
b) detecting complex formation, wherein complex formation indicates gene expression in the sample.

10. The method of claim 9 wherein the polynucleotides of the composition are attached to a substrate.

11. The method of claim 9 wherein the sample is from pancreatic tissue.

12. The method of claim 9 wherein gene expression is compared to standards and indicates the presence of type I diabetes.

13. A vector comprising a polynucleotide of claim 2.

14. A host cell comprising the vector of claim 13.

15. A method for using a host cell to produce a protein, the method comprising:
a) culturing the host cell of claim 14 under conditions for expression of the protein; and
b) recovering the protein from cell culture.

16. A purified protein or a portion thereof comprising an amino acid sequence selected from SEQ ID NO:49-62.

17. A composition comprising the protein of claim 16 and a pharmaceutical carrier or a labeling moiety.

18. A method for using a protein to screen a plurality of molecules to identify at least one ligand which specifically binds the protein, the method comprising:
a) combining the protein of claim 16 with the plurality of molecules under conditions to allow specific binding; and
b) detecting specific binding between the protein and ligand, thereby identifying a ligand which specifically binds the polypeptide.

19. The method of claim 18 wherein the plurality of molecules is selected from DNA molecules, RNA molecules, peptide nucleic acids, mimetics, proteins, agonists, antagonists, and antibodies.

20. A method of using a protein to prepare and purify antibodies comprising:
a) immunizing a animal with the protein of claim 16 under conditions to elicit an antibody response;
b) isolating animal antibodies;
c) attaching the protein to a substrate;
d) contacting the substrate with isolated antibodies under conditions to allow specific binding to the protein;
e) dissociating the antibodies from the protein, thereby obtaining purified antibodies.


SEQUENCE LISTING (62 sequences)

SEQ ID NO: 1 - DNA, 790 bp, Homo sapiens
  misc_feature: Incyte ID No 2045674CT1
ctgttgctcgagcccttagcaatatatacgtaaacatatccagcttgtctaacacatcacagattattagttaacaaggtgtagattaatgagcttatattgtattgctggatcttttgagttaataacaatggtaacttgtccagaaggcctatcatcattcctagtaggtgggcacagagtaagagatattaagaagcttcctgatgagtcatcatctagcgaaggccctgtgtagggctttattataggagttacattgacttctggggcattcaaaggtctcccctcttatccatatctctgtcattttgcccacctactaggaatgatgataggctttaataacaatggtaacttgtccagaaggcctatcatcattcctagtaggtgggcacagagtaagagatattaagaagcttcctgatgagtcatcatctagcgaaggccctgtgtagggctatgttataggagttacattgacttctggggcattcaaaggtctcccctcttatccatatctctgtcattttgcttctccagccacgacaacacactttcctctccaactgctccctccccaccaaaaaagaagaccctctaaaaggcaaaggaataaatattcttagaagtaaagtatcttcatacatgctgcctttttcaaagaggtgttaggatatttatcctatttctgtatttcacagtagcttttcaggctgtcctgcttatgtataagctgatttctcgtgccgaattcttgcctcgagggccaaattccctatatgatcgt

SEQ ID NO: 2 - DNA, 459 bp, Homo sapiens
  misc_feature: Incyte ID No 188552CT1
ggcacgagctgacatgagtctcagtgccggcaaacacggctggttgaaccctgagctagcccagctgctttgttcaccttacgtttggggaaggctgaaattttattgagcaccgactgtattccacacactcttctaggtgcccgaaatatgctgttaaacaaatactcagccctcatggggctgagagtctggtggggaagacctgttgaaaaacaatcatattaaatgaattgcattgcatgttagaagatcgtaagtactctgggggaaaatgagagtagaacaggataagggggtgatggagggaatgagtggtgattttaaatgtagttatcaggctgggcacaatggcttacacctgtaatcccagcattttggaaggccaagacgggcaggtcacttgaagtcaggagtttgagaccagcctggccaacatggtgaaaacctgtctctact

SEQ ID NO: 3 - DNA, 517 bp, Homo sapiens
  misc_feature: Incyte ID No 465676CT1
gtggccagagccagccagcatggccaccctcaagaggcgagatgagcccacagaggcatatcctgcggggatgctgggctcccagtgtggttggcctgaacaaaataaagtgttgactcctgggcatctgtgccttctctatggccttgctacctgggattccagagagttgatggggtgcagataggggtaggactgttagaatagaaccaacccaaactgtgtgtagtttggggtgtatacttctatttctcttcctacatgtctacatgccatgaccttcctcctcctcttcacttggccagtttcagctcacttcctccaggaagtctttcctgatatatcaaactgaaacaaatgctcctcctccatgctcccttaatccccatgcttgtcgattatattcctttgccaattcatttctctatcctgtctatgtataagtgtgtacaagcattcaagaaactgatgaatgatgaatgaatgaatgagccaaagaacaaataaatgagcccct

SEQ ID NO: 4 - DNA, 824 bp, Homo sapiens
  misc_feature: Incyte ID No 3601719CB1
gtttaagttcccctccagccccgagccaggagcagttctcaataccgggagaggcacagagctatttcagccacatgaaaagcatcggaattgagatcgcagctcagaggacaccgggcgccccttccaccttccaaggagctttgtattcttgcatctggctgcctgggacttcccttaggcagtaaacaaatacataaagcagggataagactgcatgaatatgtcgaaacagccagtttccaatgttagagccatccaggcaaatatcaatattccaatgggagcctttcggccaggagcaggtcaaccccccagaagaaaagaatgtactcctgaagtggaggagggtgttcctcccacctcggatgaggagaagaagccaattccaggagcgaagaaacttccaggacctgcagtcaatctatcggaaatccagaatattaaaagtgaactaaaatatgtccccaaagctgaacagtagtaggaagaaaaaaggattgatgtgaagaaataaagaggcagaagatggattcaatagctcactaaaattttatatatttgtatgatgattgtgaacctcctgaatgcctgagactctagcagaaatggcctgtttgtacatttatatctcttccttctagttggctgtatttcttactttatcttcatttttggcacctcacagaacaaattagcccataaattcaacacctggagggtgtggttttgaggagggatatgattttatggagaatgatatggcaatgtgcctaacgattttgatgaaaagtttcccaagctacttcctacagtattttggt

SEQ ID NO: 5 - DNA, 969 bp, Homo sapiens
  misc_feature: Incyte ID No 305781CT1
ccctttttttttttttttttttttttttttttttttttttttttttgggagtatagattatgtttattttctctataatttccagggttttccaaaattttacaacaaacatctataattttatgaacactccccatcttatttttaaaaagaaaaaagttggggggcagagaaatgcccagctcagtactgagatccatcaagtgaggccagccggtatctgtcacaccaggcagaggccccgtgctggaagccctggaggttaccagctccaagcctggtatccaaggcctcctgggcagccttagcctcctccttccctttcctcccaccagaccctgctcctgggatgtccttctcccattaccaccacaaaatcggactaatttttcagggcccaacaccaattctgctaatttttttttggctcaatcttggctcatcacaacctccgcctcccaggttcaacggattctcccacctcggccttctgaatagccgggattacaggcacctgccaccacgcctggctaatttttgtatttttagtagagaccgggttttgccatgttggccacgctggtctccaactcctgacctcaggtgatctgcccgccttggcctcccaatctccttccatttattagttggattgcttaaaaaaaaaaaagactccccgatatgggcaggagcaatgctgattttttacttacctgtctctagataatgaattgattgttagcctccaaagatgatcaatttgtttttgtttttgtttttgtttcagattacggtgaactcatggacttaaacttctttatgggttttgagccactgcaattatcctcaccaaatctcaagctgtcccacctctggcacgtggggcctcttcaagttttcctcattcatatttgtttgtctgtttgttgtttttgggtggccagcaggagagcatccacagtctgtctc

SEQ ID NO: 6 - DNA, 597 bp, Homo sapiens
  misc_feature: Incyte ID No 971441CT1
aagaggtaagcgtggcctgacctagccacccaccaacaggaataatggctgaaaaagcggggtctacattttcacaccttctggttcctattcttctcctgattggctggattgtgggctgcatcataatgatttatgttgtcttctcttagaaaggcaagaagatatcagattgacatcatttagaagaattaagaaaactatgaacatgactgattattaaatgtctcatgttaaacaatgcaatgtttgacatcactttacaaacttggatcataaactggcactttggtatgcataagaatttcttcaggacaataagaaattatgagtgaatttctctatattctgagtgagaaaaatgtttagctgtgatgaaaaatgcatgtcattaaaaaaagtttgataaatttaatcacattacaaaaaattatccccccttccctctggaaaaaactatagagaaagtgggctgaggctgtgcaaggtggctcatgcctgtaatcccagcactttgtgaggatcctttgagcccagaaattggagaccttcctaggcgacagagagagaccccatctctacaaaaaaaaaaaaaaa

SEQ ID NO: 7 - DNA, 1918 bp, Homo sapiens
  misc_feature: Incyte ID No 3445829CB1
cagcctgccacttgcctccctgcctgcttctggctgccttgaatgcctggtccttcaagctccttctgggtctgacaaagcagggaccatgtctacctttggctaccgaagaggactcagtaaatacgaatccatcgacgaggatgaactcctcgcctccctgtcagccgaggagctgaaggagctagagagagagttggaagacattgaacctgaccgcaaccttcccgtggggctaaggcaaaagagcctgacagagaaaacccccacagggacattcagcagagaggcactgatggcctattgggaaaaggagtcccaaaaactcttggagaaggagaggctgggggaatgtggaaaggttgcagaagacaaagaggaaagtgaggaagagcttatctttactgaaagtaacagtgaggtttctgaggaagtgtatacagaggaggaggaggaggagtcccaggaggaagaggaggaagaagacagtgacgaagaggaaagaacaattgaaactgcaaaagggattaatggaactgtaaattatgatagtgtcaattctgacaactctaagccaaagatatttaaaagtcaaatagagaacataaatttgaccaatggcagcaatgggaggaacacagagtccccagctgccattcacccttgtggaaatcctacagtgattgaggacgctttggacaagattaaaagcaatgaccctgacaccacagaagtcaatttgaacaacattgagaacatcacaacacagacccttacccgctttgctgaagccctcaaggacaacactgtggtgaagacgttcagtctggccaacacgcatgccgacgacagtgcagccatggccattgcagagatgctcaaagtcaatgagcacatcaccaacgtaaacgtcgagtccaacttcataacgggaaaggggatcctggccatcatgagagctctccagcacaacacggtgctcacggagctgcgtttccataaccagaggcacatcatgggcagccaggtggaaatggagattgtcaagctgctgaaggagaacacgacgctgctgaggctgggataccattttgaactcccaggaccaagaatgagcatgacgagcattttgacaagaaatatggataaacagaggcaaaaacgtttgcaggagcaaaaacagcaggagggatacgatggaggacccaatcttaggaccaaagtctggcaaagaggaacacctagctcttcaccttatgtatctcccaggcactcaccctggtcatccccaaaactccccaaaaaagtccagactgtgaggagccgtcctctgtctcctgtggccacacctcctcctcctccccctcctcctcctcctccccctccttcttcccaaaggctgccaccacctcctcctcctccccctcctccactcccagagaaaaagctcattaccagaaacattgcagaagtcatcaaacaacaggagagtgcccaacgggcattacaaaatggacaaaaaaagaaaaaagggaaaaaggtcaagaaacagccaaacagtattctaaaggaaataaaaaattctctgaggtcagtgcaagagaagaaaatggaagacagttcccgaccttctaccccacagagatcagctcatgagaatctcatggaagcaattcggggaagcagcataaaacagctaaagcgggtaagtaaccagagaacagacataggggcacagataaagtaaatgagttgtcctccattgcatggtggtaccaaagtcacctctcacaatacttatcaatactttcaatattttagtatgcgagagcaaacacaccaagtttgaaacattaggagcaggcacacaagtgagcacatttctatttgagaggaacgcctgggccgctttcccagg

SEQ ID NO: 8 - DNA, 1079 bp, Homo sapiens
  misc_feature: Incyte ID No 189299CT1
gtcaagctctacctgagcgacaaccacctcaatagcctgcctccggagctggggcagctacagaacctgcagattctggccttggatttcaacaacttcaaggctctgccccaggtggtgtgcaccttgaaacagctctgcatcctctacctgggcaacaacaaactctgcgacctccccagtgagctgagcctgctccagaacctcaggaccctgtggatcgaggccaactgcctcacccagctgccggatgtggtctgtgagctgagtctccttaagactctgcatgccggctccaacgccctgcgtttgctgccaggccagctccggcgcctccaggagctgaggaccatctggctctcgggcaaccggctaactgactttcccactgtgctgcttcacatgcccttcctggaggtgattgatgtggactggaacagcatccgttacttccccagcctggcgcacctgtcaagtctgaagctggtcatctatgaccacaatccttgcaggaacgcacccaaggtggccaaaggtgtgcgccgtgtggggagatgggcagaggagacgccagagcccgaccctagaaaagccaggcgctatgcgttggtcagagaggaaagccaggagctacaggcaccagtccctctacttcctcctaccaactcctgaggagcttcagttgcaagtcaatgccaaggacccaactgcagcatgttctggaagcctctccattggagtggaaaggatggctctgggtcatttgggagtggctctgctagtagagactgatggagagagccaggtggaatgccataaatcacactgagaaaatatttctggcaaacagctcctctttcagaggggagttgtgtgcccaatgatggcatgacaaatccagagatcataacttcctttgcgaagaagaacagctcgtccacagcattgtatttttggagacacttgaaagagccaaaagaggggcttgggaaacatcctgaaacctccctggaagtctctcaggaaatttgacttgggcattggaggctccattgggctccttccaattaaggggtgtt

SEQ ID NO: 9 - DNA, 1028 bp, Homo sapiens
  misc_feature: Incyte ID No 2396760CT1
gtactgactcactatagggaatttgcgcctcgaggcaagaattcggcacgaggggctgttaccaggacaaccggagcgattgaccgttatctgcggtttggagccgttagcgggagaggcagagatattcagaggtcttttaggatgtgctaaagggtcgtgagggctctcttaaaattttcttcacaagcggttatccagtcgtgccccgcggccctgctgctggccccggggatctgagtcgtaccctcttgtttttctctgagtcagtcttaaggtgaaatgaagtgtggcccagtggctcctcactgtcgcttctctagttttctgcctccttttagaaaattgaattgaaaagacaggatgaagtggacacagcatgtgaagacaattctttcaagaagtttggctgtcaaggaaaacagagaatgtgctaaagaacatacagacacagagcagacaggccacctttgcaaccacatggaggtttgtctgatattgaagctaaagaagctaagctggaagacagagagaccaagtcctgatgacattgtttgaacccagagatccagacatgcctgaaaactagttttaccactggacttatccgttgaatgagccaataaactctcttttatacttaaccttgggttttacctggatttttgtcattgacagctcaaaatattctaatatagaagtatacatcattaaatcaacatttctttttttctctgtcttattttaaatgtaactctataaggtactctaaaagtattctacagtctcactaagttaatctgcaaatttggtaaaattccaatattaatcccaaaagtattttaagagcttgtttttgttgtttgcttgtttgggactaaacagaattactccaaaattcattgagagaaaaaaaaaacatgaaaaaaaaaacaagaaaatagaattcataaaaggaaattgtattatataacaaagcataaaacaagaataataaacatagagtggtaatggaataaatagaacac

SEQ ID NO: 10 - DNA, 1149 bp, Homo sapiens
  misc_feature: Incyte ID No 919893CT1
tcgttctcactgagcacgatattaggctctctcccaactcactctattctgtcctcactcctgttttgatttttctcttgccatgtttgaaatgttttatgggaatgtattagaactcttttcttctaaggactgagacttccaggggattgccatcttacctgtctcttctccatgagggagaaggaagcagctagctatgtccctagctgcaggaagcccctattttttccaagcacgaagccaccagtctcccccagggagcatcaggaagggacatggatgtgctcctgccacagggcccttcctacctttggatctgtgagaaggtgaatacaaagcagcaggcagagtaaaatctgctgggactgcctggagatttgtcaggagctgcagacaagtaccttggagcattctgttatttttggaaagttcaaatatgcagggacaaggaggttgctgactgtactgacaggctctaagtcattttctccaaaaactatctattcaattatcaggggctggtcttgaggaaggaaaaaaaaaaaaaacgttcccagaattcagtttccaaaatctctttttaaagggtttacacacacacacacacacacacacacacacacacacacacacacacgatcattaaaaagtgtatgctctttaagaagaaaagtaaaatatctcaaaggacggtttcaccaccgtcctttattgaatcaatttttctacatttcagagcaagtgtagattctgagggactcctatttgccaaaaagacaaaactagcaaaaaaaaaaacaaaaaaacaaaaaaaaaaccacttaaaaggtagcaggaaaagaaggtagttttgagtgtggttcactcagtgtctgtgagtctggtgtagtgtcaggagtaaggccgtgtctagctcaagtttacatttggatgtcctacaacactaaacaaaatttttcataatccatggtggggagcacactttggagctacatttcttgtctcctcattgttgacattaattaaacatttataggccaggcacagtggctcacgcctgttatcccagcactttgggaggccgaggcaggtgaatcacctgaggtcaggagtttgaaaccagcctggccaatatggtgaaaccca

SEQ ID NO: 11 - DNA, 1467 bp, Homo sapiens
  misc_feature: Incyte ID No 2837330CB1
ctaaggcttatagattgccagcctgctcagcgtctctaacccttttcaggtctctgctggtggttctgaagccaaacctctgatcttcacatttgtccccactgtcagaagactaccaacccatactcagttggctgacacctctaaattccttgttaaaattccagaagaatcaagtgataagagtccagaaactgtaaataggtctaaatccaatgactacttgaccttgaatgctgggagccaacaagagagagaccaagcgaaattgacttgtccttcagaggtcagtggaacgattttacaagaaagggaattcgaagcaaacaaacttcaagggatgcagcaaagtgacctcttcaaagctgaatatgtccttattgtggactccgaaggggaagatgaggctgcaagcagaaaagttgaacaaggccccccaggggggaattggcaccgcagctgtccggcccaagtctctagctatctcgtccagtctggtctctgatgtagtgcgtcccaaaacacaggggactgatctcaagacctcatcacatcctgaaatgcttcatgggatggcccctcagcaaaagcatgggcagcaatacaagaccaagtcaagctacaaggcttttgcagcattccctacaaacacattgcttttggaacagaagactcctacaactcttccaagagcagctggtcgagaaaccaaatatgcaaatctctcctcaccaacttctacagtatctgagagtcagctgactaagcctggagtaattcgcccagtacctgtaaaatccagaatattactgaaaaaagaggaggaagtctatgaacccaaccctttcagtaaatacttggaagataacagcgacctcttttctgaacaggatgtaacagtccctcccaagcctgtctcgctccatcctttatatcagactaaactctatcctcctgctaagtcactgctgcatccacagaccctctcacatgctgactgtcttgccccaggacccttcagtcatctgtccttctccttgagtgatgaacaggagaattctcacaccctcctcagtcacaacgcatgcaacaagctgagtcatccaatggtggctattcctgaacatgaagctcttgattccaaagagcaatgaagttggagcagaggctgaaaacacaggctgctgaagttttttggaatgctggtgctaaccacttgctagatttaacttttttttttttttccagaatgagtgctccctttatgagtgcagtgcagcagaaccaaaaaaaaagtttgctgcaattatatagcatcacagtgctctgctaacagccagcatagaagagatttacctacagctttttgcaccactgttctagcctttaatgccttctacttaatattaagctgaccgcaatactaacgtgcccctatatttggcagccaaataaagaagaatcgtg

SEQ ID NO: 12 - DNA, 1691 bp, Homo sapiens
  misc_feature: Incyte ID No 1737459CB1
cggctcgaggagaaagaggtttttaaattctccatgaagtgtactatgttccatcattccttcccaaagccaccggaagcattccttctaggaaaggtggagtcggtagtgagaagccggaggtgagaagacccctgagcggatggattcattcattttctgaatttcctatgtgaggacagtattagagcccagtgaggctttgagaggccccaaagatgagcgccaacagtagcagagtgggccagcttctcttgcagggttcagcgtgcattaggtggaagcaggatgtggaaggggctatctaccacctagccaactgcctcttactcctgggcttcatggggggcagtggggtgtatggatgcttctatctttttggcttcctgagtgcaggttacctgtgctgcgtgctgtggggctggttcagtgcctgtggcctggacattgttctttggagcttcctgctggctgtggtctgcctgctccagctggcacacctggtataccgtctgcgtgaggacaccctccctgaggagtttgacctcctctacaagacgctgtgcctgcccttgcaggtgcccctacagacatacaaggagattgttcactgctgtgaggagcaggtcttaactctggccactgaacagacctatgctgtggagggtgagacacccatcaaccgcctgtccctgctgctctctggccgggttcgtgtgagccaggatgggcagtttctgcactacatctttccataccagttcatggactctcctgagtgggaatcactacagccttctgaggagggggtgttccaggtcactctgactgctgagacctcatgtagctacatttcctggccccggaaaagtctccatcttcttctgaccaaagagcgatacatctcctgcctcttctcggctctgctgggatatgacatctcggagaagctctacactctcaatgacaagctctttgctaagtttgggctgcgctttgacatccgccttcccagcctctaccatgtcctgggtcccactgctgcagatgctggaccagagtccgagaagggtgatgaggaagtctgtgagccagctgtgtcccctcctcaggccacacccacctctctccagcaaacacccccttgttctacccctccagctaccaccaactttcctgcacctcctacccgggccaggttgtccaggccagacagtggcatactggcttctagaattcctctccagagctactctcaagttatatccaggggacaggcccctttggctccaacccacacgcctgaactttaaggatcattggactatcttctctgtggccagcgcagctctcttctgtgttcacagaatggccactgataggcacgcctcttttcccacccactggaaggctcacaggcaaggtgagagaggacacagaaggtgccaacactgtcgctacagtaaggacctgaagtgactttgagaaattcaccctcacaaaccttccttcaggagcaggcattggtagtgcagaggcacagattccgtcctttaccagctgcagaatcttgggcaagttacatagcctctgtgagcctcatcggtaaacagtgggggttatgaaacccacctcacagggttgttgtgaggatccaatgagttgatttaggtaagcacctagcacatgcc

SEQ ID NO: 13 - DNA, 2379 bp, Homo sapiens
  misc_feature: Incyte ID No 058201CB1
cccaggatctgctctgaaaccaggtctctaagtgaacatttctcaggcatggatgcatttgagagtcaaattgttgagtcgaagatgaaaacctcttcatcacatagctcagaagctggcaaatctggctgtgacttcaagcatgccccaccaacctatgaggatgtcattgctggacatattttagatatctctgattcacctaaagaagtaagaaaaaattttcaaaagacgtggcaagagagtggaagagtttttaaaggcctgggatatgcaaccgcagatgcttctgcaacatgagatgagaaccaccttccaagaggaatctgcatttataagtgaagctgctgctccaagacaaggaaatatgtatactttgtcaaaagacagtttatccaatggagtgcctagtggcagacaagcagaattttcataagtcctgcttccgatgccaccattgcaacagtaaactaagtttgggaaattatgcatcacttcatggacaaatatactgtaaacctcactttaaacaacttttcaaatccaaaggaaattatgatgaaggttttggacataagcagcataaagatagatggaactgcaaaaaccaaagcagatcagtggactttattcctaatgaagaaccaaatatgtgtaaaaatattgcagaaaacacccttgtacctggagatcgtaatgaacatttagatgctggtaacagtgaagggcaaaggaatgatttgagaaaattaggggaaaggggaaaattaaaagtcatttggcctccttccaaggagatccctaagaaaaccttaccctttgaggaagagctcaaaatgagtaaacctaagtggccacctgaaatgacaaccctgctatcccctgaatttaaaagtgaatctctgctagaagatgttagaactccagaaaataaaggacaaagacaagatcactttccatttttgcagccttatctacagtccacccatgtttgtcagaaagaggatgttataggaatcaaagaaatgaaaatgcctgaaggaagaaaagatgaaaagaaggaaggaaggaagaatgtgcaagataggccgagtgaagctgaagacacaaagagtaacaggaaaagtgctatggatcttaatgacaacaataatgtgattgtgcagagtgctgaaaaggagaaaaatgaaaaaactaaccaaactaatggtgcagaagttttacaggttactaacactgatgatgagatgatgccagaaaatcataaagaaaatttgaataagaataataataacaattatgtagcagtctcatatctgaataattgcaggcagaagacatctattttagaatttcttgatctattacccttgtcgagtgaagcaaatgacactgcaaatgaatatgaaattgagaagttagaaaatacatctagaatctcagagttacttggtatatttgaatctgaaaagacttattcgaggaatgtactagcaatggctctgaagaaacagactgacagagcagctgctggcagtcctgtgcagcctgctccaaaaccaagcctcagcagaggccttatggtaaaggggggaagttcaatcatctctcctgatacaaatctcttaaacattaaaggaagccattcaaagagcaaaaatttacactttttcttttctaacaccgtgaaaatcactgcattttccaagaaaaatgagaacattttcaattgtgatttaatagattctgtagatcaaattaaaaatatgccatgcttggatttaagggaatttggaaaggatgttaaaccttggcatgttgaaacaacagaagctgcccgcaataatgaaaacacaggttttgatgctctgagccatgaatgtacagctaagcctttgtttcccagagtggaggtgcagtcagaacaactcacggtggaagagcagattaaaagaaacaggtgctacagtgacactgagtaaaatatctatggccactgacagtccacacttaggcactgagagatattgatgttctgaaataagattttatgaatttggatacccttttgaggaacttgatgtaaacatggtgttcagaaatctcgtgtctatctcaatgggatatttcttgtattacaccttgtcatttttttcacaatttatttacatctacttttgtttgaactggaatgaagagatgaaacactatggatatgttttccattcaaatggcactttagcatattgttctgttttcctgtaaaacatcatgggtgtgatttttatactgctgctgcttgtcacaattattataacttctctgtaatttcctctgaaataaaattgaatcacctgaggtgcaaaccaaaaaaaaaaa

SEQ ID NO: 14 - DNA, 1904 bp, Homo sapiens
  misc_feature: Incyte ID No 767447CT1
atgaatacaaatcgctcagaaagcattttggtggcacagaaaggggatgtatttgtgttgagatcttattttattttgtatttatttatcttctttgacttgcacagcactattgggggtgggggaagcagggtagtgggagacgaaggcagaagcaagagtcaaactcagaatgactgagttgaattcactgtctagtcagcaatgcctgcttctgagtttggcccagagagaaggtattgagtaagattttaataactgtaaaaagtaagctggataagtaaaatcatgatggatccaaagcacagtttcttcatctcctgataaagaaagtcaaatgcttgataaattcagagtcacagatgtgagcatagctatattcttttaaacgagaggtagagtgacctagcactaagcaaatgagctgaaatgtcggaaacagagtccatcagcttatttggccacacgatcccaaactagttttatcttgggaaatggccctgtcctcagcattcccttcttgtgctggtggggccagtgaagtcttgatcttatcagaaaaaggccacaccaagtgcgagttttcccaggctgactttccaggcccttatcaaatgaaacaacagaagctcttcacagttctgtgccccatggccactccacagacagacaataccaagcatcttagaactgtcataagataggtcatgcctgaaatagatcttgaccatatgagagtcccagaaatcagcaaggcctggacaaatagaactaagagagaggcagaggcaggaagctgcgggtctatcttgtaaagagtttagcatcactgtgagagtgtgtgtctaaaattaaattaaactagaagcagcaggtgagtatttggtaagtacttctgtgactcgcctcaattcccactggccaggggccatctcaactgcacggtgaatcaagatgctggtgtcatcctccttggaaaaaggaaatgttaactcatggttaaaactaagtacaatgattcccaagggatcactttcttatttttttaaatgacattaaggagaatcttaagaaagcatcagagaaagacatgtgcatgtgaagcaccctgattctgatgttaggaaaacttaagcgaacaggacctgctgcacacagccccattgtcttctatccatttctctttatcattcaaatcaagcaacatgtgccctcctcatcaacacacattcttcccctttgtcagtatgcatctcccagcttagtgtcaggatactttcgattcataattatgtatgatccaaagtgtgcataatttcatttaacgttaaagaaatagatccaattcctttcttgcaaccaaaaataaataaaatacgttgcctcaatataaggtttgggctattctgtgtttctatagaagcaatctgtttttggtaaaatgtacttttaaggatccagtcatctgaagtattttatgtagagttagagatttcacaatattgactatacatatatttaaaatataaattatccagctgatgtttgaatttgtcttactttcctggccacctcgttgtcctattttataagctggggagttaactagcttaacaaaagatgcttagcttttgtaaaagaacaagtgtttcattttacaaagacactccaaatgatagttacttgattttctcgagacctttaactatggtgatgaataacaggacttgctttcaagccttaataaatgtaaaatgccttttaatgaagatacagctgagtgttttcctcatgaatctgaaccaattaccaatttgtgttccagtcttgattggtattgactgattcaaataaagttggtttattttcaaatattaaaaaaaaaaaaaaaaaaaaaaaaaaaa

SEQ ID NO: 15 - DNA, 968 bp, Homo sapiens
  misc_feature: Incyte ID No 5449893CB1
gaatccaggggaagggatggaagaggaagaaaaacaagcttggagcagtccaacccagctagggccctccattccctcagggacaccccacacccaccccacacactgggatgaacccttgcagaggaacaattcagatggtcacacattccaggacccaaatccgtaaacacaaagcatgtccgtcagtgccagcacctccccccggctaatcaagcagctgtcccagagggcaaagggtctctgcagccatctgctttcatcagggctgcagcccccaggcagcagtactgggagcccctctcatctccgagaataaactctgaagccagcgaccctgcggacctgaatcatcagggagcctgtcagaggaggggcagtgactctgcgggacaagcaagcaggctatataagtttcagaaggctgggctccactcagatcttttccagcagctgctgcctgccagagaggcgccttcagagacccagcgcttacacaatacccaccatgtcccaggctggtgctcaggaagcccctatcaagaagaagcgcccccctgtgaaggaggaggacctgaagggggcccgaggaaacctgaccaagaaccaggaaatcaagtccaagacctaccaggtcatgcgagagtgtgagcaagctggctcggccgccccgtcggtgttcagccgcacccgcacaggtaccgagactgtctttgagaagcccaaagccggacccaccaagagtgtcttcggctgagaagtgtgcgccactccccttgctgcccgaatgctcggaaacaggagccttacccaggaactcttttttatgccagaacgcttcctctcccctgctgtctctggggctgccaccctcccccacagtccaggcccttcagccaagggctctgcaccagcaccttggaagcaccaataaagaggatgcccacgtggccccagcaaaaaaaaaa

SEQ ID NO: 16 - DNA, 1112 bp, Homo sapiens
  misc_feature: Incyte ID No 2951269CT1
gaggcaagaattcggcacgaagggtagacctcacaggtgcataaaatcattaataaagcatgtagcacttgctaattggtgccttaagcttgaatctaatcagaattgcagactcgggtcctctgggaaaaaaacatgtccgtctgtggcacgtgtgagtactaggcccaggggaagagtctgaaaattgaattcttttgtgtgtcctgtgtctcagaagagaactgaatgttcagagcagcgtttgtaagctattaacattcagtatttcgtgttgcaactagaacacattattagatttattcctgtttaattcataatggtgcagaataaaacacacacatctgatttgatttctttttctttttttaagtttcataattgctttttatggctagtgttaatggcaaaaagtcctttccagggctccctgaataatctaccatacctgtatccatagcaggtgatgcttttttttatccccactttgaagacgtgtgtttctgtatttacacataaatcatactattgtatattaaagacagcagtggttgaaaagaatgtgaacactgtagaagttatgttggaaaaaaggagagtaaattgtgtgattaatggggaaggatattggataatgttataccccggactatgaaaaaagctggtggtaaatgggaagaatgtgaaattttaaactgctctcaacgtaggaatcttggtggaaaagttcctacctgaggtctgatatgattcaattatagaatgcaatgagcttggccaaggggactttgaatccagccaaggaaactttgaatctcgacagctctgagaatcacattttcagtgcattgaatatggagtaaactatttagacaaggattctgtgagactaggctacttacctttaattgccagcatttgtaaatgattgtgcaatcttgtgtaatggtcttttattttgactgttttggaaaaaaaatgttttattgtttttttttcccagtaaaaattacttcaaagaaaaaaaaaaaaaaaaaaaaaaaaaaaaaaaaaaaaggcggccgcaagcttattccctttagtgagggttaattttagcttgcacttgccgt

SEQ ID NO: 17 - DNA, 1714 bp, Homo sapiens
  misc_feature: Incyte ID No 282977CB1
ggaaagtggaagttggattctgaaagatcgaggtgcccacaggaattttatggtcgtcggattttgaagacttgaactagactgggggttctccttgcatttcttgcctgttgcctatctttgtcctctctcttccggcttcgagatgaatgtgcagccctgttctaggtgtgggtatggggtttatcctgccgagaagatcagctgtatagatcagatatggcataaagcctgttttcactgtgaagtttgcaagatgatgctgtctgttaataactttgtgagtcaccagaaaaagccgtactgtcacgcccataaccctaagaacaacactttcaccagtgtctatcacactccattaaacctaaatgtgaggacatttccagaggccatcagtgggatccatgaccaagaagatggtgaacagtgtaaatcagtttttcattgggacatgaaatccaaggataaggaaggtgcacctaacaggcagccactggcaaatgagagagcctattggactggatatggggaagggaatgcttggtgcccaggagctctgccagaccccgaaattgtaaggatggttgaggctcgaaagtctcttggtgaggaatatacagaagactatgagcaacccaggggcaaggggagctttccagccatgatcacacctgcttatcaaagggccaagaaagccaaccagctggccagccaagtggagtataagagagggcatgatgaacgcatctccaggttctccacggtggcggatactcctgagctgctacggagcaaggctggggcacagcttcaaagtgatgtgagatacacagaggactatgaacaacaaagagggaaaggcagtttccctgcgatgatcacacccgcctatcagatagccaaaagagccaatgagctggcaagtgatgtgaggtaccatcaacaatatcaaaaagaaatgaggggaatggctggtccagccattggagctgagggcatcttgacaagggaatgtgcagaccaatatggccatggttacccggaggagtattaggagcacaggggacagggcagcttcccagctatgatcactccagcatatcagaacgccaagaaagctcacgaactcgctagtgacataaatacaggccggacttcaataagatgaaaggcactgcacattatcactcgcttccagctcaagacaacttggttctcaaacgggctcagagcgtaaacaaactcgtgagtgagaataaatataaagaaaactaccagaaccacatgagaggccgctatgaaggagttggtatggacagacgcactctgcatgctatgaaagttggcagcctggcaagcaacgttgcctacaaagctgattataaacatgatattgtcgactacaactacccagccactctcacgccttcctatcaaacagctatgaaactggtgcccttgaaagatgccaattataggcagagcatcgacaagttgaagtacagctcggtgactgacaccccacagattgttcaagccaaaatcaatgcccagcagctgagtcatgtgaattaccgtgctgactatgagaaaaataagttgaattacacattgccccaggatgttcctcagctggtgaaggccaaaaccaatgccaaactcttcagtgaggttaagtataaagaaggctgggagaagacaaaggggaaaggat

SEQ ID NO: 18 - DNA, 806 bp, Homo sapiens
  misc_feature: Incyte ID No 3178454CB1
acttgtctcagtctggatcagactcaagttgctctccagaatgcctctgggaggaaggcaaagaagttatcccaactttctttagtaccatgaacacaagctttagtgacattgaacttctggaagacagtggcattcccacagaagcattcttggcatcatgttgtgctgtggttccagtattagacaaacttggccctacagtgtttgctcctgttaagatggatcttgttgaaaatattaagaaagtaaatcagaagtatataaccaacaaagaagagtttaccactctccagaagatagtgctgcacgaagtggaggcggatgtagcccaggttaggaactcagcgactgaagccctcttgtggctgaagagaggtctcaaatttttgaagggatttttgacagaagtgaaaaatggggaaaaggatatccagacagccctgaataacgcatatggtaaaacattgcggcaacaccatggctgggtagttcgaggggtttttgcgttagctttaagggcaactccatcctatgaagattttgtggccgcgttaaccgtaaaggaaggtgaccaccggaaagaagctttcagtattgggatgcagagggacctcagcctttacctccctgccatgaagaagcagatggccatactggacgctttataagaggtccatgggctggaatctgatgaggttgtatgatggctgctgggcagcacctcctaacttcagggaataaagtgctaaagtgtaaaaaaaaataaaaataaaaataaataaataaataaaattaaaaaaaat

SEQ ID NO: 19 - DNA, 555 bp, Homo sapiens
  misc_feature: Incyte ID No 3563859CT1
gccagacacctgagccgactggtagtaggggcagccgtgtggcggggagccggccgggccttcctgctcatcgaggacctgactggctcctgcttcgagccactgccccagggtctgctgctccacgagctgcctgaccgccgcagctgcctggcagccggccaccagtggcgaggctacaccgtctcctcccacaccttcctgctcaccttttgctgcctgctcatggcagaggaagcagctgtgttcgccaagtacctggcccatgggcttcctgccggcgccccactgcgccttgtcttcctgctgaacgtgctgctgctgggcctctggaacttcttgctgctctgtaccgtcatctatttccaccagtacactcacaaggtggtgggcgccgcagtgggcacctttgcctggtacctcacctatggcagctggtatcatcagccctggtctccagggagcccaggccatgggctcttcccccgtccccactccagccgcaagcataactgaaagaaataaaaaccatcgggcctgaaaaaaaaaaaaaaa

SEQ ID NO: 20 - DNA, 1159 bp, Homo sapiens
  misc_feature: Incyte ID No 985730CT1
taagatctactcaaagtacttcaaacaaaaaataaataattcattggctcatgtatcttggccacccagggaaggtctgacattgttagttagatccagagtttcaaatgtcatcaccatggatgtgtctttttctctctctcatttcccctccccatatcttgtcttttatttattataggtttgtctcattccctggcaggctctctccctgtgataggaaagagagtccccagcagccccagggtgacatagttgttatagttcattatggaatagaagagagaagagcattctcaataacccggcaaagttcccagggatgactctgatatgtctatgtctcaggtcacatttccatctatgaaccaatcatattcagaggtggaatgctaattggccaggcctgggtcatatatacaagtctagggaagaaatgagcttcatccctgtccaattgacatggactgattaggggtattaatggaagaggtgtgccaccacaaaagaatgtaccatgggcagatcaaagaacatattctgtatgtcaggcttggcacaaaagaatgacacaagtaatatgctgtagatcagaacctctctgctaatattgcctttttagcatggttaagatagctaagatctagtactgtcactccagtatgtcccaattctacctacgtttattgaagggtcaacagttctgatctcagcattgggtaaagggtgggacattcagatttacggtccttgataaaaacaatttacaacgttccgttgtgtaataaatgtaagtgtacatatgcctgggacatcagctggaaaagggacagactatcagagagttgcactgttgcggtatgggccaaatccaacataatacccgctgtacctctagagaactaaaaccttaatttctcagatcttttctgcactaatggtctttacatacagcctacattttaactaactcttgcatgggcttgtttcacagcaggaaactatattcatcatatccttattatgatagagaatgacaacattcaaaagggtgtggtgcttctgaaaatatacacaataaatggcatgatttgaaaaaaaaaaaaaaaaaaagatcggcgcaagcttattccctttagtgagggttaattttagttga

SEQ ID NO: 21 - DNA, 878 bp, Homo sapiens
  misc_feature: Incyte ID No 3684987CT1
gtggcatccaccattaaggttaagtgtggtgtgccctgtgagtctgaatgtctacttaagaaccttaagtagacattaagaaccttaagaaggttttttgtttgtttttgtttttttgttgttgagatggagccttgctccgttgcccaggctggagagcagtggcgcaatctcagctcactgcaacctctgcctcccaggttcaagcaattctcctgtctcagcctcccgagtagctgggactgcaggcgcctgcccccaagcccggctaatttttgtgtttttagtagaaatggggtttcaccttgttggtcaggcttgtctcaaactcctgacctcaggtgatccacccacctcggcctcccaaagtgctgggattacaggcatgagccaccatgcctagcccacaaactcttaccattcttaaatgtatttatttcagttcctcttccactactatattataacctaccctggcagtccttctcatctgctgcaatatttcccattccttaagatctaacctatgctgctccttctccatgaggctttttctcattaattcatgcacactgatctctcccttctctgcattcctgtcatacatcattatttcataattattttgcatgtgttgtactttttcttttcagccacattcataagtctctggggaaagaaattaggctttcatgattttgtatccttatcctacacccggcaaagtgctgagtatacagtaaattctcaaaggctttatgtcttcttcaatcgaaaaatttacacttgaagaaatttgtcttgtagcctatgaagtcaaacagtaccattaggaaacaataatcaagactccatgacctaaccatgttatattatta

SEQ ID NO: 22 - DNA, 667 bp, Homo sapiens
  misc_feature: Incyte ID No 986166CT1
gcgttcaggagacagtcacggtactcgtttccagacagaagtcatgaggaacaagagggaaggtgctttcccgtgtgcagcgcttggggagactcacacagacagaggatctggcatgacagggaaaggaggaaatggcttctgttaatctctccttcagcttctcccgcccttcccatgcactcttcctgtttccctttccagttctcacggtgactcaaggaacaacgtgtgaaatgaaagacctcaggtgctgtattggctcttgacagctcttcagaagaaaatacctcctgcctgttctgttcagtcctggtgcagcttccaggaagccaaatgacccaccggcttacccacatcgcaggaagctttggagcagagtcagtgactatgtgaacctgcctcaacctctgctccctggttcagcatttggcttgggaaaaatgacactatttcctgtctcttaaacattatttcaaggcacaggtcttccaccattctgagaggcagggggatctttgagttctgccaggagctgggggttaggggtaggggaatcccgcccaagggaaatgactagaatctttgtcaggctgtggaacacaggcattctggataggtggctcccctgtggctctccctggaatctacatgcaaatccctgtat

SEQ ID NO: 23 - DNA, 1421 bp, Homo sapiens
  misc_feature: Incyte ID No 1887508CT1
tgatcagtgatatcaaacatcaggaatcagcctttatgtaacataacagctgtcctcctatggtgaaaggttcaaatgtagtgaaggtataacctatattgactgagatttcccttttaggtagtgccttatctctattactagtgttaaaggaataaggaatctatgaaggacagggagcagctctggtctgtcaatctcagccacctgtttgatatcacagagaagatactcggaggattgttggaatgtatatagtttagtaagaagtgggtaagaaagagggtcttaattactgagcacttattatgtattaggttctttgccagatgtttttacatatataaactcatttcagaaaacttatttaaagtaaatggggccgggtatggtggttcatgcctggaatcctagcactttgggaggctgaggtaggaggactgcttgaggccgggagttggagaccagcctgagcaacatagtgagaccctgtctcaataataataataataataataatagtaataatgaagtaaatgggataaggaaagaaggataattatctttaaaggttgattcccaccctccctccccagttacttaaggaactaagtgagtacatctccagttgcccatgaaagcataagtttgttttcctcagctgaggcaagtggtagagtatacaggataacgaagtaacatgtaaaaggcaggacgcacataaaggtgtacatggctattgtttcacctggagaaaccacatgattgggacctgaaggtttactgactgactacaggggctgattgtgaagcacgaggaaccccatgtgtgtggagactgtagggtgagagcacacaattattagcatcatttctgagtgatctcacagattttttttcttgtgtttgttttgctttttgacaactgcttctcccacgttccttgcaattctattctctcaccttcactttactatttgtattcgatggaccaggataattcaggcaaggttaccttgtaaacttgaattggccacacaccatgttgtcacccagctggctatgaagtgaataatggtactgaaagtaaacctgaagacctttctcagatctattttaagtctgagtctgaccaaccatggaaaatattcgacatgaattaatgtagagaactataaagcatttatgacagctccaagaaaagtcatctactctatgcaggagatatgtttagagacctctcagaaaaacttgcctggtttgagggtacacagtaccattttaatcttctgaaaatatctgtattcctgctctttttctgctgtcactgtcaatctgctatatttttcactatcctattaaaatattactgtctccttaaaaaaaaaaaaaaagggcggccgttcgcgatctagaactag

SEQ ID NO: 24 - DNA, 2630 bp, Homo sapiens
  misc_feature: Incyte ID No 1006416CT1
aataaaggagctccaaatgtcgttgggtggggaagcaaaatgtagagaaacatttaaagcacactgtaataataaatgcaattataaactatatggaggagggtgcagaggagggaatgtgtctggtgtgtgatgtgtgtgtgtgcagtgggggtatcacagagagtatgacatctgagttgagggtagcaggtgcctggagtctcaggtggctgctcacccatctgtgcaggtgtctctggggctgctggtctcacctgtggtctgcagtagacacaattggctgagcaggatatgtgatactgtgtggttggtgtggagttttgaagaaggggctgtgtttgggccacgtaggctctactcagagacctgaaaccacttcagaatggtgcatatgtcgaaagagctggctgggggccttgcccaaaccaactgaggtcttaaagtccggggaaaaaaagtctgggttccaactagaattctagaaatatttctagaacacacagagagggaataagtccctctatcacccttattaccaagccttgtggttccctgtgattttagataatgtctgatatttttctggctatttgcctagtaggatttaaaaaatattttcaaagtgaagctgagagagaatcttggaaacacacatacctgttgatcatgggccctgcagaattggcccttgggggctttatttggttacatgtgcctgggtggtctttaccagcttagactctatcatgggcccccatgaagctccattctcaatactgaataattattacttcccttgttgagtttctttttctgtcatgccctgggggcttctgctcttctcaccagaaagaacatttgaatctggattcttgtacacctgggttagaccctgttcagaggtgtggccaatttatcccgatctcctggaaggctgttgtgatttccatctaagaaatgagggtcttgagaatcaaccagtcccaagattagcctgttatcctgttatctactgagacctcaaatttctcaccaatgttttgggagatcctggaaaagatcccttcagtttggggtgtcaccaagacttctacacaacccaggactaccattgacctcagagctgtaccccacatcttgaagtaaattgatcccaccaggtcccacgtttgttatctctgcctaaatgttagcttctccatcctcaccacatgatgacctgctgtgtccctctgagcactacccagtggctgaaaactctgcaaatgggccacacttttgcaaaatacttgtatctgacacttaggtcttgtttgaagaatttcctttctggaaggttttacaagaagactgatagtctttcaagcccccacatcacaggcttagggacggcactaactttctcccagggatctaactggctagttcaaattatcactcttttaccttcatataaaatgtctcccccaaacctttttcccttctttgtcattgttatctgctaagccactggtcatttccccatattcgtagtctttttttccatcctatctttctaatatttgttgtctttaacaaactgtgttctgtgtctgtgctcctccttccctctcagaccactggaatgcaagtccttcttccctttggaatgtactctggatcccttcccctgctttgacccccagactttgctccatctattattgcttctccatcctggatccttgacatttgtcaccccactggccttctcaggtgcaatcagtaaaaatgctgagaactcttggatcttaatcttcatgactgagttttttttagttgtatagttatcatctgcctttcttcactttgcatttcttcttgaatccattgcagattgacttccactcccactccttcactaaaagggctcttaccaagatcaaatctaatgggtacattttagttcctatgtgatttggcctttcgatgtcaatcatcactcccagccattgattttggtgacccacttccctgtgatgatcttctgatctagtttctcaggttccttcgctggtcctttttctttccctgcccctgacatattgacatttcctggagttggttttgtccttgattcattctcatgtcattctgcacacagtctctgcatgaactcaggcagacccttcatttaatgaccaccttagggctgatgattctcaaatctgtattccccgatcttgcatttgagctccagccccactcatcctctcggatgttctgcaggcccagcaaactcatcatgtccaaagtgaaactttttctctttcctgtctcctctcctctgatctgttctttcttggaacaccacccaagaacgtcacctcctccatcagattgtgagctcctggagggcaggagctgtgtccttctattcatcttcctatccccagaaccttgcacagatcctggaatgtggtaggtgctcagtaaatgtgtgttgaataaatgaatgaatgaatgaacaaatgaatgaatttgcttacttcaaggcaaaagaaccatgaaactgtattttgagtttctatgttatagcagtcagcaaatcctattaaatactttgtgtttccaaaaaaaaaaaaaaa

SEQ ID NO: 25 - DNA, 1039 bp, Homo sapiens
  misc_feature: Incyte ID No 975169CT1
gttgacacgttgtatgccatcctggatgagaagaaaagtgagttgctgcagcggatcacgcaggagcaggagaaaaagcttagcttcatcgaggccctcatccagcagtaccaggagcagctggacaagtccacaaagctggtggaaactgccatccagtccctggacgagcctgggggagccaccttcctcttgactgccaagcaactcatcaaaagcattgtggaagcttccaagggctgccagctggggaagacagagcagggctttgagaacatggacttctttactttggatttagagcacatagcagacgccctgagagccattgactttgggacagatgaggaagaggaagaattcattgaagaagaagatcaggaagaggaagagtccacagaagggaaggaagaaggacaccagtaaggagctggatgaatgagaggcccccagatgcagagagactggagagggtggggaggggcccagcggcccttggtgacaggcccagggtgggaggggtcggggcccctggaggggcaatggggaggtgatgtcttctctctgctcagagagcagggactagggtaggaccctcaccgctgcgtccagcagacactgaaccagaattggaaacgtgcttgaaacaatcacacaggacacttttctacattggtgcaaaatggaatattttgtacatttttaaaatgtgatttttgtatatacttgtatatgtatgccaatttggtgctttttgtaaaggaacttttgtataataatgcctggtcattgggtgacctgcgattgtcagaaagaggggaaggaagccaggttgatacagctgcccacttcctttcctgagcaggaggatggggtagcactcacagggacgatgtgctgtatttcagtgtctatcccagacatacggggtggtaactgagtttgtgttatatgttgttttaataaatgcacaatgctctcttcctgttcttcaaaggaaaaaaaaaaaaacaaaagggaaaaaagggagagaaaagag

SEQ ID NO: 26 - DNA, 1057 bp, Homo sapiens
  misc_feature: Incyte ID No 4152861CB1
ggagtcgggttacaccacttgtgtctgagttcacgcagcatgttcctctgtcagggattccgcaaatatctccctgaggtaaaaaaggaaagtgtgctgcgctccagcacccagagcagtgagcccagtcccgagtcccggagagagctccagcaataggggccatgtcgccatagccccagcctctcggtccgcagcctcagcagcgtcccagccggctggcttcatgctgcggtgcagctgcaccatgttcctgggttgagggggcaatcgggcacgctcctccccatgggttgcccatcatgtctaatggatatcgcactctgtcccagcacctcaatgacctgaagaaggagaacttcagcctcaagctgcgcatctacttcctggaggagcgcatgcaacagaagtatgaggccagccgggaggacatctacaagcggaacactgagctgaaggttgaagtggagagcttgaaacgagaactccaggacaagaaacagcatctggataaaacatgggctgatgtggagaatctcaacagtcagaatgaagctgagctccgacgccagtttgaggagcgacagcaggagacggagcatgtttatgagctcttggagaataagatgcagcttctgcaggaggaatccaggctagcaaagaatgaagctgcgcggatggcagctctggtggaagcagagaaggagtgtaacctggagctctcagagaaactgaagggagtcaccaaaaactgggaagatgtaccaggagaccaggtcaagcccgaccaatacactgaggccctggcccagagggacaagatctaaaaaaaataatgctgggaagtcctaaccacatcaagaatgcctcagatcagtgacccaaggaaccttccagaatggatgaaatagacccaaagctgaattcacctaattttagggccaaaaacccaaaaaacaaaacaagaccaaaaaaatcttcagatactgggagaacaaatctcaattgctcaattgtatcttatgaaaacaatttttcaaaataaaacaagagatat

SEQ ID NO: 27 - DNA, 1363 bp, Homo sapiens
  misc_feature: Incyte ID No 986464CT1
gaaatcacacagaggccagaggtcacacagcctcaactgccccttccaccaggaggcaggagacatcaagagagtatttgtgccctcctcgggttttaccttccagccgagattctccctcctccccaacatttatctccatccagtcggccacaaggaagcctctagagactcccagctttaagggcaaccctgatgtctcagtgaaaagcacacaactggctcaggacataggccaggccctgctccaccagaaaggtgtccaagacaaaactgggaagaaggacatcacccagtgctctgtgcaacctgaacctgcccctccctcagccagtcccctgcccagagggtggcaaaagagtgttctggagctacagacggggccagggagctcacaacactatggagccatgagaaccgtgactgaacagtatgaggaggtggaccagtttgggaacacagtcctcatgtcttccaccacagtcaccgagcaggcagagccacccaggaacccaggctcccacctcgggctccacgcctcccccttgctgaggcagttcctgcacagcccagctgggttcagcagtgacctgacagaagctgagacggtgcaggtgtcctgcagctactcccagccagctgcccagtgaggcccaccgcctcccaccacacctgccacctgttcctggcctccactgccccaggactgaagtgggtacctgcctcctgtacactggagcaaggaccaagaggaaatggcatcttcagaggattactgtgggccatttccctttcgcagttctttcaataggcccagttcttccaaatggaaaaagaaaggtctggaagaggcccacagagttgcacaggcgtgggggtaggatgggggctcccagctgcttgtggaggatgtaatatatacagacacacacatgtttttcacacaggcctggcccacgcatcgacatgtgtgaatttgcacaccactgcctgaattggagccccccagagtgtccctctacccagagtttttatttctttaattagtctgagtgttcccagccatctgctccttaatccctggagaggaacagagccaactggacacagcgttggtctctgtttggaatcactgtgaggtctccagaaggacctggccgccagccccttcatcaccatctccatcattcagctggtcatctggtggcccaaaggtcacccaaagagtcagcaatcagcatgtccctagaagccaaatgcactgcctttctctgtccccatgactgtcccccactctgcaccccaaatgggaagcatacggtctgaataaatccaagttttattctctaaaaaaaaaaaaaaa

SEQ ID NO: 28 - DNA, 1513 bp, Homo sapiens
  misc_feature: Incyte ID No 118472CT1
cttcaacatgcccctcactatctcccggatcacaccaggcagcaaggcagcccagtcccagctcagccagggtgacctcgtggtggccattgacggcgtcaacacagacaccatgacccacctggaagcccagaacaagatcaagtctgccagctacaacttgagcctcaccctgcagaaatcaaagcgtcccattcccatctccacgacagcacctccagtccagacccctctgccggtgatccctcaccagaaggtggtagtcaactctccagccaacgccgactaccaggaacgcttcaaccccagtgccctgaaggactcggccctgtccacccacaagcccatcgaggtgaaggggctgggcggcaaggccaccatcatccatgcgcagtacaacacgcccatcagcatgtattcccaggatgccatcatggatgccatcgctgggcaggcccaagcccaaggcagtgacttcagtgggagcctccctattaaggaccttgccgtagacagcgcctctcccgtctaccaggctgtgattaagagccagaacaagccagaagatgaggctgacgagtgggcacgccgttcctccaacctgcagtctcgctccttccgcatcctggcccagatgacggggacagaattcatgcaagaccctgatgaagaagctctgcgaaggtcaagggaaaggtttgaaacggaacgtaacagcccacgttttgccaaattgcgcaactggcaccatggcctttcagcccaaatccttaatgttaaaagctaaaaggctgcctggaatccccccaccccaacaggctggactccctccatccttacccccacacagatctggcatgtgagccccacggtgatgcttgacaatgtataactctgctgggggcacctctgatggccaaccgcagcatttctgtcctctgcccaccccagagctgatgctggggcccagccccctgcagctctgtacccaccaaacctccccagggcaaccctcgccaccccccaaatagcccgtagcccaatcccctgccctctgcacagggccttagctgtagaccagagagggcaggaggggtttgctggcataacaccccagaaccaagggaaatggatgggccgctgctcagtttcccaccatcctcagctcctggcctcatcccctcctagaatgagtcacccgtagatcagggtctggggaagaggctgatccctggcgctgcccggctccctcgctgccctctggagctcagggcagcccggaatagggctctttgaagaggaagtagaagccccagggtaatgaggcagagacccctcctggcagtggtgaggtgggggcatgcaccctcctttctgtaccgtgtgtgctggctccatagttctctcttctgtacatataagcatgcttgttctgaaataaagaagatttgaagtgaaccacaaaaaaaaaaaaaaaaaaaaaaaaaaaaaaaaaaataaaaaaaaaaaaaaa

SEQ ID NO: 29 - DNA, 627 bp, Homo sapiens
  misc_feature: Incyte ID No 1314633CT1
gcctgtgtgagcctgaagacctttctgtcctggcgacccctcagaagggctgcactggatcttgtctgcccggggagcgcacctatccattggagggaagagcctcctgcgggtagaggatggccagctactcagcaaactggacttgagggggcctgggcagctggagccctgctctgaggaagaagcacattccctgaagcgtctggaagatcagagccctgggccaccaagggggtggcctgcaggaagagccccttcacggagaaaccttgctcagaatccctgcgggtgccagtggagccgcttttcgcctttggggcattctggactcagcttgggctgctgctcccgacccctacccccagccccatgcccgcgcttcccctgctgtgtgtagtgggagatctctctgtgcctggcagcccctgcagaccctgggagggagctcaggctgagccaggcactgcaggggatctgggaaagccaagatgggcaaggaaacccttctatggccaggagtggtggctcatgcctgtaatcccaacactgtgagaggccaaggcagaaggatcagcttgaggtcaggagttcaagaccaaggggggcagcatcgtgaagagaagg

SEQ ID NO: 30 - DNA, 1606 bp, Homo sapiens
  misc_feature: Incyte ID No 1997439CT1
ctcgtagactcgcattgacttaatttttttaaatcttattgcatattttgactagataataaatgcatatggttaaaaaattcacatggttcaaaaaagtacacctcccactcatcttccatgtgatatttcctttctgcttagcaattctgtatttatcttgctaaacatgaatgacagttgtttgctgaaattacattaaatgtgacgtaataaaatcattgtaagtatacattttttaactttaataatttttaatgtcttaatgaagagtatgaagagtagtagtactgctcttcaaagtactactactttaccttaccttttactgttttgttaagaaaattaggccgggcgcagtggctcacgccggtaatcccagcactttgggaggccgaggcgggcggatcacgaggtcaggagatcgagaccatcctggctaacacggtgaaaccccatttccactaaaaatacaaaaaattagctgggcgtggtggcgagcgcctgtagtcccagctactcgggaggctgaggcaggagaatggcatgaacctggaaggcggagcttgcagtgagctgagattgcgccactgcactccagcctgggcgacggagcgagactctgtctcaaaacaaacaaacaaacaaaagacccaatctgagtcttatcgttgtactgatagaagggtcagatatccccacatggagttgagtgggagaaagagattcactagagaataactccttagagaccaatgtctgtagcaggtgtacagcatcttgtgaaagttatggagcatgaaaagactgaagggccaggacagtttgcatgggctgagttataccagctagaccaggaatagaacaaagaattctatacctcaggatttcaaaaagttagcaacttgagaggccagtgctgagcaacccagtacccaggaaatgaaaaaaaaaaagaaaattccctccgagaatgaacaaatcattggcttcattgcctcatgagcttgagagaaaggagaagagagccagagtgtggcaagtgaggccaaaatcagaagcatggcagaaatgagtgtaagtgattgagccacagacagaagtgtggcgagggacaatgccatattgggagaaggtaaagttgagtaacaagaaaccaaccgtgtgtgagagggggattggaaaaaaatttgagggagaagaatgttagaatggaagggaatgatggtggaagggaggtgtgagggtgtgtgctgagtgttgaaagaacggttggtgtctgtgtgattttccttgagtctgttcttcagtgtgtcttctgcagcttgccatgactgcctgggaaagagtagggaaatacccagagccaaaacctcctttcagtcccaccccatccctcaaaaccccagctattgcttcttttcagcttcaggtcctgatctccaatcttagtatggactcccttctcaccaagaccaccaccagctacgtttgctgtgtaatctggaaagtgataatttcctttgcttgttgggtgtgagtcacaatactttggtttgtgcacaagaataaatttatgccccataccttcaaaaaaaaaaaaaaa

SEQ ID NO: 31 - DNA, 2184 bp, Homo sapiens
  misc_feature: Incyte ID No 2638878CT1
gccaaatggattgagtgatgagcagacatgtttaagggtctaagtctcaagaatctgttatgtgtgtttgctgcggtgggagggggtgcttgtatttatcttatttccagtcactataaggttgtacacaaactaatttaaagtttacttaataatggtatctttaaaataattgacacaattgcaaaatgaattcctggcttcagttagctattatttttttaatgacaacatagactgtgctctaagtttaaaagatggggaagcttatataaaagtgacccttttgcatcatatgggtatctaaacttaatttacccaataagttgatgcttaatgattttattttatttttgtctatttctattttagttgtggctttgctctaagaatgggtaatagttgtactacagactgctataaatttcttgtgatactcttttagagctcaaaatatctctgagctttagacatggtaaggtggagagtaaatgcttgataaatctttaagatatgtcttgaatgataattaggacattcagtccagtggaaatacaccattcaattagtcaggtctggtgaatcgtttgtttaaaatattagcaaatgagatgtggaattctgaaatttctccagactgtgtcttaataaaaatgtcacctgggtgaaattttagatcaatcactaaatttgggtgacaaatataaaaatattttcatttcactttaatacattctttctgtgaagtaaaatgtttttctttctcataatggcaaaatatgaatgccatcaaagtttaaggaattcattttagccttaaatgccttcgtgagatgtcttacttgtattttaggtaactggtcatcagtgccaatgacatggataacaatttttaatctactcgacagtgcatccctgggaatgactgttatgtttttgtcatattcctggtaatataaatactcgtgttctttactacattgtttttatcaactctaaaagtcatgcctctgtgacctttatcatgtttacaattgcaactgaacttatgacaaattaactcaggaaataaattgagttatcctttctagcattgtaattaccatcagcaaggcctgagatagccagagccaatactagccaagtgatttattttcaaggattgccactaactacggttctttaggaccaagatataaaacagtcactaaaaatcattaggctaggtatcagtaatacattcattactaataatgcatttttggagacttttgtgaaagaagttggtctctgccaaaagctggtggaccacattcacaccacgaaagccagtgtcacatgaaccagattaatgactctctttatggggtatgtgggacatcctggaagtgtataatttcaggaatgaccagacaataccatcttgcaaagccccttcaggtgacaatctaaacttgtgggtaggagagtgcataaagtttattgctcaactgctcctccagcctgctgaatttactgagtaaagaaatagcaaatatgatagatgttttagatttcatagaacagaatggtttgtccattaattctttcattcaatgactgtttattgaatacctactcttttagggcgctgtgttaggtgctgtattgtacaagaaaaatataataaattagattcccagcgctattctgacatagtgaatgaccttgaaaaatttactaaacatactatgtttgtttctccatgagtaaaatagggatatagggacaaacagtctaatatctcatagaaataccatggagacaaataaaatattttaatataaatatgatattaaagtaaatttctgaagtaatacttttgggtatggcactagtttttcctctgactattttactgtttctttcactctcaatataaaaactatttgataagataaaacgatatattttattgtaattagaatttagacaaatcagctataatgtaaaaatgttaataataattacgttttatctgattaaagttacaatgatcatagcactttaaaaatattatctgaactgtcatttgtttatatattaccgtctaataaaatagttatagatcttccaagtttgatgccttacattttaaaaggaaaagataaatggttgattaagaaaaaaaaaaaaaaaaaaaaa

SEQ ID NO: 32 - DNA, 1833 bp, Homo sapiens
  misc_feature: Incyte ID No 3795510CT1
cgggcagtgcaagctaaaattaaccctcactaaagggaataagcttgggccgccattttttttttttttttttttttttgctctttagaagaggttatatttttattatccttattttggagaacttttccttataaaattttttttccagattccttatgaactcaagttagtgttaaagctttggattccactgttaacagtttatgtaaaaacacttaacaaattgccatttatatgccaaactatagctcaagaacactctgttttagaaaaattacgcattagatcaggaagcctcatatatatgtgcctctgggacttcatttgcagtcacatttagccagaaaagcaatgacttctatattccttatggaaaccaatgtaacataaattaatgttctaaatatagaaattaagagttcataaagagactgaggttgcatgtaaaagagttatggtttgagacagtctaaaaatactatgttaatttcaaggatcttatttccaatgttttgtttaaaaaattataaatacttttgagctcttgctttgcatttcaatcgcaaacccactcagatacgggaactgtttaaattcatatatggacaaataggtttcagtgatgcaatactttaaaattctgccatctccttgtgtttttctttctaggtgagtggactgccagctcctgatgtgtcatggtatctaaatggaagaacagttcaatcagatgatttgcacaaaatgatagtgtctgagaagggtcttcattcactcatctttgaagtagtcagagcttcagatgcaggggcttatgcatgtgttgccaagaatagagcaggagaagccaccttcactgtgcagctggatgtccttgcaaaagaacataaaagagcaccaatgtttatctacaaaccacagagcaaaaaagttttagagggagattcagtgaaactagaatgccagatctcggctatacctccaccaaagcttttctggaaaagaaataatgaaatggtacaattcaacactgaccgaataagcttatatcaagataacactggaagagttactttactgataaaagatgtaaacaagaaagatgctgggtggtatactgtgtcagcagttaatgaagctggagtgactacatgtaacacaagattagacgttacggcacgtccaaaccaaactcttccagctcctaagcagttacgggttcgaccaacattcagcaaatatttagcacttaatgggaaaggtttgaatgtaaaacaagcttttaacccagaaggagaatttcagcgtttggcagctcaatctggactctatgaaagtgaagaactttaataactttaccaacattggaaaacagccaactacaccattagtaatatatttgattacatttttttgaaattaatccatagctgtattaacagattatggttttaattaggtaatatagttaatatatatttataatattatttatcctttgactcttgcacattctatgtacccctccgatttgtgaagcctacaggaaatctgggtatatggatttgtaactgcagaagactatcttaaaatacaggattttaacatttaagtcatgcacatttaacaattacaggttataaattagtatcaactttttaaacacatctaatgcttgtaataacgtttactggtactgctttctaaatactgttttacccgttttctcttgtaggaatactaacatggtatagattatctgagtgttccacagttgtatgtcaaaagaaaataaaattcaaatatttaaaacggaaaaaaaaaaaaaaa

SEQ ID NO: 33 - DNA, 1859 bp, Homo sapiens
  misc_feature: Incyte ID No 1413537CT1
cttctctttcctgagcctctttagagcagcacttacaggattgcctctgtaaagccttattcctgtcccagaaaaggtaatccaaaaagtctctagtatccactaaaaggtaacccaaaaatctctagtatccactggctttctccagtgtggaagctttcccctccacctcccatagatcactggaaaggacccgaggcctcggttctaatccctggcttatcactaactgctgtgtggctttggcttgtcccttagtctctgtgagactgctgcaccctcatctgtcaaagatggaactggacttagttgagctctgaggtccctgtggacttggcccctccacaccctcattatggcaactggacataaacttaacagagggcttcccagcaaaatgtcctcttcttcctacaaacaggctgtttctatatgtgcatgtttcatgctaagcacttctttcttgggtggagatggcaaaggcctctttctgctgagacaaagtgatttggagagtcacctggcccctgaagggggagtggtaggatccagccacccagtgtgcagtgaattggagcagggatctcagcacacagggaggtggggaggctccccctaacctcgggcacctgttgctcctccagactgcagcgcatgctcttagctcatcctcttaactggctctcaccgtgctcctggctttggtcaccacgtagctctcactccagcttcaggtagccatcagtaggacctggcaatatacactgatttggtttgttttatgtttgtctgcaggtgaaatccctaagggctctgccgtgtactccagccttgtgacccttgccttccaggaaccatgcaagaagcgcagccaccagaagtccttaaaacagcaggaaaggtgagcctgtcccccttttgtgcagctacctatctgctgaggagcatctgggcctcattcctccaagtccactggagggtccagaagagggagtcagagatgtatcctggtggagctgggagaaaggcagaaagcctttgtgacagctatggaataccgttagccaaggtccacttggcccagcactaagcaaaagatgcgtagtttgcacagaaggttttgtgatactgcctctcaacagccccagcagcttgggaactagcaagagcacatttcttgcctcatcagctgtcctgagatggaaaactcagtggatataggaccctgattccgatgaaaggggcacgtggtcccaatgctggagctcctctggcaggttctaaaagcacactacggagcagcggtgccctgccggacactgctggcgggggctcagtgagcactactcacagatccacacctgaccctgttgggtcgagtcaggctgggctttggtctgcactgtagcacctgtgttctttgagttcacatcatgaatgtggtgatttcccagataccatctcaggcttaacctagcacatcctatttcttttcttctatgatatccaaattggactgacctcacttcaaagttgctgtcccattttgtcaccctatcttatctcggggaaattgcagactgatggccagaccaactctgttgaaattcttgcatagagcaaacctgtgctcatttttaagtggcatgggagaggccccaagcctagtaaagcctagtctgtgtcttcacagtgctggtagaatgtgtttgtgtgtataaatatatgatatagatttatatatgttgctaacgccacatattgaaggccaacataactggtggacagggtgggtgacagaaaatgaaagtctttttggtgattgtttaagcaagatgtgtataaagaaataaatagtt

SEQ ID NO: 34 - DNA, 2125 bp, Homo sapiens
  misc_feature: Incyte ID No 1623157CT1
tgtgtaaacaataacaagaagacatgaaggatttatttggttatcaactgcccatggaggaggctcttgatgatcccaggtctcctcgacctccatacaccacacaggcatttgtaagcacagtttccacaagcaccttgtaggaatatggataagattagaccagcccctctctgtccactgggtttatttcttgaagaagatgcagatctggtttttccaatgtgccacagtctttccttatcctctccatgctgagcttgacaacactctgggaatgaggaacaagactttttctaaaaagatagtggaagttcaagggatgtacctcgttttcaggttcatccatctccagtggaatgttttcaataaaagatgaagaaaatgtgtgtgatctttaataacacatccctatagaaagtggataaaagatataccaaaactgtaatacagatatatacaaatataggtgcctttttgattactcttgtttgtctagtatggtcttggaaagaaaaccaagcaagcaagttgctgcctattctatagtaatattttattacacatgattgatatttttgtggtagggaagtgggatgctcctcagatattaaaggtgttagctgattgtattttatctctaaagatttagaactttagaaaatgccgacttcttccatctatttctgaaaggttctttgtggatttatatagagttgagctatataaacattaactttagatttgggatttaaaatgcctattgtaagatagaataattgtgaggctggattcactacacaagatgaacttcacttcataaattaattataccttagcgatttgcttctgataatctaaaagtggctagattgtggttgttttggttaaggtgatatggaggtgggagagcttttagttaagtaagaagctatgtaaactgacaaggatgctaaaataaaagtctctgaagtattccatgccttttggaccctttcctcgcaactaactgtcaactgttgatcaaaaaagtcaaggcattgtatgttgcttctgtggttattattctgtgatgcttagactacttgaacccataaacttggaagaatctttgagcaaattttctcagttgtctgtatgacttcagtatattcctgggaatgccataggattttttgtgcttgatacatggtatccagtttgcatagtatcacttctttgtaatccagttgctgttaagaatgatgtactttaaaggaaaagagaaaactgcatcacagtcccattctccagtgtccatgcaatgaattgctgagcatttaggaagcagcaccaagtctattacaggcatggtgtgaaacttgatgtttgacctgtgatcaaaattgaaccattgtacagtttggcttctgtttgcttcaaaatatgtagaattgtggttgatgattaatttgcgagactaactttgagagtgtaacagttttgaagaaaacattgaatgttttgcaaatgaaggggcttcacggaatgttacaatgttactaatataatttggcttttgttatgcaaattgttaacaccagctattaaaatatattttagtagaaatgctttaattcatatttttttcctctacactgtgaatctttaagccttggtggactagagcaacatcgtgctgcccaaaggactaacctatgcaaactagttcacattttagtggatgtcgcagttaatgtgtaataagacattatttcccctgcataatgtacaacagcattgaaatgacacattaagcctagcatcacattgtatagtacagtcactcacaaacccttcaaggctaccctaatcattaacattaatatttgtttaaaagcaaatcaccgatttatctattgaaactacttaaatgacggcaaaccaggaatgacagatggctgtgtcagcaatggctttaatgtgttccctgcaagtggtctcctatgatagaactgcgttctcaaatgcactctcttcagggtcttaatattctgtgttttctctctgtatttgtaaaacattataacacattaatttcctatctctacacatttggtttgcttaaataaatgcaggatataaa

SEQ ID NO: 35 - DNA, 1686 bp, Homo sapiens
  misc_feature: Incyte ID No 3009303CB1
tctgactgccagcaccttacagagaagagaccatcaccactgtggtgaagagcccacgtggccaacgacggtcccccagcaagtccccctcccgctcaccttcccgctgctctgccagcccgctgaggccaggcctactggcccccgacctgctgtacctgccaggtgctggccagccccgcaggccggaggcagaaccaggccagaagcccgtggtgcccacactgtatgtgacggaggccgaggcccactctccagctctgcccggactctcggggccccagcccaagtgggtggaggtggaggagaccattgaagtccgggtgaagaagatgggcccgcaggtgtgtctcccaccacagaggtgcccaggagctcatcggggcatctcttcacactgcccggtgcgacccccggagggaccccaattccaacaactccaacaacaagctgctggcccaggaggcctgggcccagggcacagccatggtcggcgtcagagagccccttgtcttccgcgtggatgccagaggcagtgtggactgggctgcttctggcatgggcagcctggaggaggagggcaccatggaggaggcgggagaggaagagggggaagacggagacgcctttgtgacggaggagtcccaggacacacacagccttggggatcgtgaccccaagatcctcacgcacaacggccgcatgctgacactggctgacctggaagattacgtgcctggggaaggggagaccttccactgtggtggccctgggcctggcgcccctgatgaccctccctgcgaggtctcggtgatccagagagagatcggggagcccacggtggggcagcctgtgctgctcagcgtggggcatgcactgggtccccgaggccctctcggcctctttaggcctgagccccgtggggcgtcaccaccgggaccccaggtccgtagccttgagggcacctccttcctcttgcgggaggccccggctcggcctgtgggcagtgctccctggacgcagtctttctgcacccgcatccggcgttctgcggacagtggccagagcagcttcaccacagagctttccacccagaccgtcaacttcgggacagtgggggagacggtcacccttcacatctgcccagacagggatggggatgaggcggcacagccctgatgctgctgccatggtggcttggggcagcggggagaaaggagtgtccttgaggcctaggacgctgcccggcctcagcagcagccctgggagcctcctgagggccctccctgtccctggccacgggcccttcttacctcactcaacttcagccaggaggactgggtggtgcttgcaatgttggaatgaccggctcaaagacctcagctctgggctgtttcctgtcagcctggcaggagcctcaggactgtggacgaaggatgtggccttgggcatttgtcctgttcccacatgggcctggtccctccctcctggccccagccacagctgccaggcctgacatggccttgcctctcctgcagtcttggtgactgagacccttgggtggcgcttcccagctctgcaggccctcctggccttttctgcagggtggacacagggtctgtgtgtgggcagcagcccctgtctctcagcaagaataaagcagcttcctgtgcaaaaaaaaaaaaa

SEQ ID NO: 36 - DNA, 2350 bp, Homo sapiens
  misc_feature: Incyte ID No 3434460CT1
cttgaaaggatcattgtgcggattaaaagaaataatatatgtaaagcactttaacacagcaccaggcccacggaaagtggctaatgttagctactatgaatggtgccagtgaagacactgaaaaataagtgatttcagtaaccttctggaaagctatcagtttcaaataatattttctctgtaatatgagatgaaattaaaagtggatagctttcaggaaagataaagagaacatgcttagaatgtaagctaaacagattttttctgttgctctttgaaaactatgagccctggccagcttaacctggtctgaggtgagactaaacacaaaaacagtagataaatctctccctaaaagatggattcccccacatacccatgctactagtttctctgtctattcacacatatgtacaaatacatgaacacagcctgtctgtgctcagacatagagaagtactacctgacttgagtcaatgcacccaagaagaaaagcttggagtagagcagaagggagggcttgggactcctgtctttccagcatgccctggggtgcagtggtcagccacctgaagagagagccaatagcatggggtttacaaggcaaagatagtcattcattcaacacatattcatagagctccttctctgtgccagacactgttctggaagatagctagatgaaaatctttgcactcacagagcttacatgccagtgagtgaagatcgatgataaataaagcaaatgcatcatatgttcacatttgataagtatatgccaaaaaatgaagccgggaaggaggacaaggcccatgggtgggtgttgaggtttttaaagtgtggtcaggaaaggccccactgataaggtaacatttgagcaagtctgaaaaaggcaaggggatctttggggctaacttcgggatccctgcactttatgtaagaatgtaaacctggagtctcatttaagaatgatcagcaatacgtttagaacatatgaactgaatgaaatggacattttttcttaatttacgtataaatccatatgattatacataaagttctgatgcattaataaaagcagccaaatagggccaaagagaaaaataacaggactctgtactggacctaactttatcattaattaggtaatattttcctcatttctttactgctgccattttcctcaccagtattccagagatggtcatagctcattactctaccaccaagaacctaaaaggaattagaatacagcagaattggcctcagtgaagagcttaaaattgttctcctcgtagaactggactattgatcattaccacgtgacgttggctctattactttctgttcccaatgtccttctagtggtttgaaaatgttaaaacatccctaaaatctaaatcatataatcagaattctatagtgtcccactctatctgtaaagatcatttggaagactttagactctattaattttaaaaggaatatttattagccatatgcagaatttctaatgatgatattgtacagcttctaattcacttttcagatcagtgtttgaaatggcaattatcagtgttggatttagttccaactacttgatttacaaaaatgtacatttagaggttaaaagaaacagtgagaaatgtaaacattcaaaatgataattgaatctctcagttgtgggaataattatcagagacatgcaactgaaaatgtctcacctttcatctttttttcttaattcataaagttatcttgtagaatttgatgagaccctcctagtcattctcaactggggcggtgctgtcaccgaatggtgtttgagagtgttggggctagggcacatttttggttgtcacagcaactggggtggcatttgctgcccagtgccaggaatagtaacattatgaatgccagggacagtgtgctcagtaaagtcttccatccaaaaggggcagggcacggtggctcacgcctgtaatcccagcactttgggaggccaaggtgggcggatcacctgatgtcaggggttcgagaccagcctggccaacatggtgaaaccctgttgctactaaaaatacaaaaattggctgggtgtggtgtcacatgccagtaaccccagctactagggaggctgaggcaggagaatcacttgaacccgggaggcagaggttgcagtgagctgagattgcaccactacactccagcctggatgacagagtgagacttcatctcaaaaaaaaaaaaaaaagggcggcagctctagaggaaccaagctaacgtacgcgagcatgcgacatcatagatcttctatagtgtcacctaattaatacatggccgtacagag

SEQ ID NO: 37 - DNA, 3502 bp, Homo sapiens
  misc_feature: Incyte ID No 5022769CT1
gcggccgctgacagcaccagcatgtcttacagtgtgaccctgactgggcccgggccctggggcttccgtctgcaggggggcaaggacttcaacatgcccctcactatctcccggatcacaccaggcagcaaggcagcccagtcccagctcagccagggtgacctcgtggtggccattgacggcgtcaacacagacaccatgacccacctggaagcccagaacaagatcaagtctgccagctacaacttgagcctcaccctgcagaaatcaaagcgtcccattcccatctccacgacagcacctccagtccagacccctctgccggtgatccctcaccagaaggaccccgctctggacacgaacggcagcctggtggcacccagccccagccctgaggcgagggccagcccaggcaccccaggcaccccggagctcaggcccacctttagccctgccttctcccggccctccgccttctcctcactcgccgaggcctctgaccctggccctccgcgggccagcctgagggccaagaccagcccagagggggcccgggacctactcggcccaaaagccctgccgggctcgagccagccgaggcaatataacaaccccattggcctgtactcggcagagaccctgagggagatggctcagatgtaccagatgagcctccgagggaaggcctcgggtgtcggactcccaggagggagcctccctattaaggaccttgccgtagacagcgcctctcccgtctaccaggctgtgattaagagccagaacaagccagaagatgaggctgacgagtgggcacgccgttcctccaacctgcagtctcgctccttccgcatcctggcccagatgacggggacagaattcatgcaagaccctgatgaagaagctctgcgaaggtcaagcacccctattgagcatgcgccggtgtgcaccagccaggccaccaccccgctgctgcccgcttctgcccagccacctgctgctgcctctcccagtgcggcttcgccacccctggccacagctgctgcccacactgccatcgcctccgcctccaccacagcccctgcttcaagtcctgccgacagcccaaggccccaggcctcttcctacagccccgcagtggccgcctcttcagcacctgccacccacaccagctacagtgagggccccgccgcccctgcacccaagccccgggttgtcaccactgccagcatccggccttctgtctaccagccagtgcctgcatctacctacagcccgtccccaggggccaattacagtcccactccctacaccccctcccctgcccctgcctacaccccctcccctgcccctgcctacaccccctcacctgtccccacctacactccatccccagcaccagcctataccccctcacctgcccccaactataaccctgcaccctcggtggcctacagcgggggccctgcggagcctgccagccgtccaccctgggtgacagatgatagcttctcccagaagtttgccccgggcaagagcaccacctccatcagcaagcagaccctgccccggggaggcccagcctacaccccagcgggtcctcaggtgccaccacttgccagggggaccgtccagagggctgagcgattcccagccagcagccggactccactctgcggtcactgcaacaatgtcatccggggcccatttctggtagccatgggccgttcttggcaccctgaagagttcacctgtgcctactgcaagacttccctggcagatgtgtgctttgtggaagagcagaacaacgtttactgtgagcgatgttatgagcaattctttgccccgctgtgtgccaagtgcaacaccaaaattatgggggaagtaatgcatgccttgagacagacatggcacaccacctgcttcgtctgtgcggcctgcaagaagccttttgggaacagcctcttccacatggaagacggggagccctactgcgagaaagactacatcaatctgttcagcaccaagtgccatggctgcgatttccccgtggaggctggcgacaagtttatcgaagccctgggccacacttggcacgacacctgcttcatttgcgcagtctgccatgtgaatctggaggggcagccgttctactccaagaaggacagacccctgtgcaagaagcacgcacacaccatcaacttgtaggcggccaaggccgcctgtgctgacgaggcccggagctgctcctgctgctggcaacaaaggattcgggaggctgatgtttcttctgaggggaatggggagagagaggaagcgactgagccctttggaagtataattttaggttttttcttctgtacacagatcgtgcatttgcatagttcagactaggagccaaatgaagactcaaaaccaagctagttattaatccaagactggaattgtacttcagacatttagagcagaattccaagaactcaaaagtgaaaagcaacaagcagctttcccaaagcgatacacttgctttggtcaccagaggaggacagagcttagagcagctgtggagaatctgaagcattctgcggagttcttaagcgctcccctggcaaacaaattgaagtgccaaacagcactcgctgcagggtatttttagagtcatagctgagagcttgttagctaagacccattgggctttcctcaccaaaaaaggaagtgttattccattactagcgtcatggagctacctctgcgcatcagacttcagaccttgaacaaacttaaaaccttcttgggagcccggacgtccaaagagatgtcttctgggagccactgggcaattgccagggctccaggaagggctctggctcaggttgcagacagctgagaaaagatggccctgtcagccaccctctctcagtctgaaacatccaacatccccagaaggcttagctcctttttgaattgtgatgggaaagtagagttgggtttttccagttttgctctgtggtgtgtgagagatttttttaaaggctttgggttgtctttggcctttgtttagctttaagggttcgttagcatgagtgtccagtcgtgtgcatgaatttcaccccaacttgtgactgctcacttatgacgtctcccccagtaccctccatctcaaataggcttggtggcctgtggaaaagaagagagacagagagacagtgtctgaaacaggatggcagaataggctcacatgcccaaactctgggtggggaagaggaaacttactttctgccaccctcagtaagaacacacgaggaggcaggacctcccaccttcaggtctgcatcatccttttcaaatgttcctttaaatgcagcacactgagtttgtacaattgtgttaactgctggaagggacagatgcactgatatatatgcatttgctgttttggccaatattttgaaaatgtatgagctgagttgatctagctattatttaagtatttattgaagtagag

SEQ ID NO: 38 - DNA, 1689 bp, Homo sapiens
  misc_feature: Incyte ID No 944140CT1
cagacgtatgaagcatccatggacaagctgagggaaaagcagaggcagttggaggtagcgcaagttgaaaaccagctgctaaaaatgaaggtggaatcgtcccaagaagccaatgctgaggtgatgcgagagatgaccaagaagctgtacagccagtatgaggagaagctgcaggaagaacagaggaagcacagtgctgagaaggaggctcttttggaagaaaccaatagttttctgaaagcgattgaagaagccaataaaaagatgcaagcagcagagatcagcctagaggagaaagaccagaggatcggggagctggacaggctgattgagcgcatggaaaaggaacgtcatcaactgcaacttcaactcctagaacatgaaacagaaatgtctggggagttaactgattctgacaaggaaaggtatcagcagttggaggaggcatcagccagcctccgtgagcggatcagacacctagatgacatggtgcattgccagcagaagaaagtcaagcagatggtcgaggagattgaatcattaaagaaaaagttgcaacagaaacagctcttaatactgcagcttttagaaaagatatctttcttagaaggagagaataatgaactacaaagcaggttggactatttaacagaaacccaggccaagaccgaagtggaaaccagagagataggagtgggctgtgatcttctacccagccaaacaggcaggactcgtgaaattgtgatgccttctaggaactacaccccatacacaagagtcctggagttaaccatgaagaaaactctgacttaggcactcagaggcatacactttttacagatggacaaaagctctggaaccctgtggcttcaaatcctttgggaagggtgactgttgtttcccctacacacagtgtaagccggaatgggaatcgctgaggctctgatccacttctaagacaggaaggaaagtgaaggcagagtgagcaggtaagagagggatatacaaggtcacatttcagacacccactcggcataccctgccgtactgcatcatcatttgttttctttgtagacactgaaatcctatcaggaggattccttcacaatgtattttatttgctagactttggttgggagggaaaaggacattaatttgaagtttcatgttattcatgccaggattgtttgatagagcatgaaggttttgtttacccataaaagtattagaggcagcgtttctctgatacagagaggcctgtccacaagaagcatgggcacccagccaaacttgaacctggaagggagggttcccggcctgcaggtgctctttcctcttggtcccaagcatctgtgcagggtcgtgggagccacactgagagacttgtgtgggccagacaagcttcattctgatgcgctagtcccttggtttaatttgtgccttatgctttcattggaccagctgaaatcactgtatttattcaacttgtgatttttttttctttctcactttaacttaaagagaattttatatgtcttggaaatttaataatttagtgttctcagtatcaattggtgtttttgttaaacgaatgaatcatctgttcatgcatgctctactttgatattataacctatgtcacatgtgtttaataaataccatatattttgttctaaaaaaaaaaaaaaa

SEQ ID NO: 39 - DNA, 1918 bp, Homo sapiens; identical to SEQ ID NO: 7
  misc_feature: Incyte ID No 3445829CB12

SEQ ID NO: 40 - DNA, 1086 bp, Homo sapiens
  misc_feature: Incyte ID No 3016490CT1
gcggccgctacggcgtcttcgtcaagatcaagtatggctccgagacgggccagggcaccattagtgtgttcaagcacggggacgagcccaaggagctgaagagcatgtgacagcgtgtgtccaggcacagtctgagtctagtctgcatggaccagtagggacaacctgtaccagggtcacagcctggcacaggctacaggggtggggcagaaggaaaggggacaagatagaacccaggatgtgagggtgggggtggagcggatgcaccaaagtggagaagcaaagatctttctggggtcctgagtggcttccaggagagcgggatgaaccctggacctggagtaggagacccggatgcactggggctatctaacagtactggcatctgataggtagaggtcaggtacgctgctaaacactgcagctcccaccacatagaattatccgaccccagatgtcaaaagtgccaagggccatgagccctgccataaactgatacatcgcacccctcttttaggatcccatagtttcaattcatgtaagttcaacagacacctgaagtctagcatgtgggaggctgaggatggagctgggaacacaaaggcagctgataagcaggttctgcttgcaaagaggcctcagtccagtgggagaaacagacctgggcgcaaacaactccaggacaaggcaggacatgataaagattataaagcaggtccaaggaaagtgccgccagtggtccaaggagggagacagagggtcgtcccaacagggggaggtagggctttgaaaacaccttcatccaggctgggcgaggtggctcacgcctgtaatcccagtagtttgggaggccaaggcgggcagatcacctgaggtcaggagttttagaccagcctggccaacatgacgaaactcagtctctactaaaaatacaaaaattagccaggcatggtgggcagtagctgtaatcccggctattcagaaggccgaggtgggagaatccgttgaaacttgggaggcggaggttgtgaattgagccagatttgggccaaaaaaaaaattggccgaaattggtgtttgggcccc

SEQ ID NO: 41 - DNA, 3441 bp, Homo sapiens
  misc_feature: Incyte ID No 4151935CB1
gtttcaaaggacacaaagagagatgtggactcaaagtcaccggggatgcctttatttgaagcagaggaaggagttctatcacgaacccagatatttcctaccactattaaagtcattgatccagaatttctggaggagccacctgcacttgcatttttatataaggatctgtatgaagaagcagttggagagaaaaagaaggaagaggagacagcttctgaaggtgacagtgtgaattctgaggcatcatttcccagcagaaattctgacactgatgatggaacaggaatatattttgagaagtacatactcaaagatgacattctccatgacacatctctaactcaaaaggaccagggccaaggtctggaagaaaaacgagttggtaaggatgattcataccaaccgatagctgcagaaggggaaatttggggaaagtttggaactatttgcagggagaagagtctggaagaacagaaaggtgtttatggggaaggagaatcagtagaccatgtggagaccgttggtaacgtagcgatgcagaagaaagctcccatcacagaggacgtcagagtggctacccagaaaataagttatgcggttccatttgaagacacccatcatgttctggagcgtgcagatgaagcaggcagtcacggtaatgaagtcggaaatgcaagtccagaggtcaatctgaatgtcccagtacaagtgtccttcccggaggaagaatttgcatctggtgcaactcatgttcaagaaacatcactagaagaacctaaaatcctggtcccacctgagccaagtgaagagaggctccgtaatagccctgttcaggatgagtatgaatttacagaatccctgcataatgaagtggttcctcaagacatattatcagaagaactgtcttcagaatccacacctgaagatgtcttatctcaaggaaaggaatcctttgagcacatcagtgaaaatgaatttgcgagtgaggcagaacaaagtacacctgctgaacaaaaagagttgggcagcgagaggaaagaagaagaccaattatcatctgaggtagtaactgaaaaggcacaaaaagagctgaaaaagtcccagattgacacatactgttacacctgcaaatgtccaatttctgccactgacaaggtgtttggcacccacaaagaccatgaagtttcaacgcttgacacagctataagtgctgtaaaggttcaattagcagaatttctagaaaatttacaagaaaagtccttgaggattgaagcctttgttagtgagatagaatccttttttaataccattgaggaaaactgtagtaaaaatgagaaaaggctagaagaacagaatgaggaaatgatgaagaaggttttagcacagtatgatgagaaagcccagagctttgaggaagtgaagaagaagaagatggagttcctgcatgagcagatggtccactttctgcagagcatggacactgccaaagacaccctggagaccatcgtgagagaagcagaggagcttgatgaggccgtcttcctgacttcgtttgaggaaatcaatgaaaggttgctttctgcaatggagagcactgcttctttagagaaaatgcctgctgcgttttccctttttgaacattatgatgacagctcggcaagaagtgaccagatgttaaaacaagtggctgttccacagcctcctagattagaacctcaggaaccaaattctgccaccagcacaacaattgcagtttactggagcatgaacaaggaagatgtcattgattcatttcaggtttactgcatggaggagccacaagatgatcaagaagtaaatgagttggtagaagaatacagactgacagtgaaagaaagctactgcatttttgaagatctggaacctgaccgatgctatcaagtgtgggtgatggctgtgaacttcactggatgtagcctgcccagtgaaagggccatctttaggacagcaccctccacccctgtgatccgcgctgaggactgtactgtgtgttggaacacagccactatccgatggcggcccaccaccccagaggccacggagacctacactctggagtactgcagacagcactctcctgagggagagggcctcagatctttctctggaatcaaaggactccagctgaaagttaacctccaacccaatgataactactttttctatgtgagggccatcaatgcatttgggacaagtgaacagagtgaagctgctctcatctccaccagaggaaccagatttctcttgttgagagaaacagctcatcctgctctacacatttcctcaagtgggacagtgatcagctttggtgagaggagacggctgacggaaatcccgtcagtgctgggtgaggagctgccttcctgtggccagcattactgggaaaccacagtcacagactgcccagcatatcgactcggcatctgctccagctcggctgtgcaggcaggtgccctaggacaaggggagacctcatggtacatgcactgctctgagccacagagatacacatttttctacagtggtattgtgagtgatgttcatgtgactgagcgtccagccagagtgggcatcctgctggactacaacaaccagagacttatcttcatcaacgcagagagcgagcagttgctcttcatcatcaggcacaggtttaatgagggtgtccaccctgcctttgccctggagaaacctggaaaatgtactttgcacctggggatagagcccccggattctgtaaggcacaagtgatccttggctttcagaatttgcaagaacagcgatttgaattttgggggggtctgctgttcattcctttaggtgctatacattattcaaaaagtctcccgcgcatttgcactaatgatggctgcatgcatagcaatcagcatgtgagcaaaatcgacaagaaaaccttgactttacagagcagtgtgtgagtaaacagaatgaaaacaacaacctccactctttagtttatataagtttgagttctttcctaaattaaaagatctacacttgagttgggaaccaaaagagaaaaatggacttccatctgttttactggtaaaggaaatcctctgatggacaggtcagagtgaaggaaggttgtgctggtaagacatctctgacgaagagccatggatgctttccacaaaatgtcacctcgctgcactaaaggatgatgaatcctaatcattaaaggaattgtttcagctgatttaaatttataatgaactcttttgtaataatgtatactgtagaacatgagtctctcctccctaaaattttaaatgtagaaaagtgctatatattagaaatttccattttgttaaataaatggttagagtctataaagccagtcatgttatgtgaacttactccatgtaacttactggc

SEQ ID NO: 42 - DNA, 1461 bp, Homo sapiens
  misc_feature: Incyte ID No 3719652CT1
cactaagaaggggctgtgctttgatcccctgcctcttgcactaccaatgtctcaagacataatattcatctcttgctgtcagacccattctatattctaaaagcttctgctccttccttcccaatttctcctttgtagcaggaaattacacccagccctcatctcaattaatgctaaataaagctattgtttttccaaaacacaaatctacactgggtctcaatatcagtgatgaggcttacaaaccaacacgttttctgccatgaggatttctctttaggccagaagtacaaaacaaaaaaaccaatggattttaaccaaaatgatttgaaatataggtgaggattcaggagaaggcaaaagctagaaacacttggggttgtcaacatgagtattacattaacattgcttgatgagaacctctaatgatactgacaacataaattacctagggtaaaggatagctgcaacaatgaaacaggaaagaagagagggagagagaggaaagggaaggaagaaaggaaggagggagaagggaagaaagaaacaatgtctaacccaaccctatcttgaaagttgaactcaagtagaaaaatggatagaaacaaaattctctagtactcatccaggaaaccattcttcaatgttgcatgtggctgtttgccaaggcacacaaagtgcttgtaggcagcaaccatatgctacaagaattgtaaactgcatacagtttgtttgaagtagacagtgaggataataacaaagttgctaggcaggaaaaaaaatcaggaaaaaagcttgtcgctatttgagaatctgtatatttttaaaggcttaaaatattataaccacagggtatccagccaaattcaacattactgcaagtcttagagatttaaacattcatttgattcatagctaaatattcaccataatccaggagggtctccttccccactgcagaggcagaacgtccaagaatggagtaagattagtcatagtaaagtctcagtctgaatatttagcaagagaaacaggcagcagaggaacccaaaggcagtaaatcaaatattctaaaacccaaagttcattattttcatccaaaagactttcacagaaacacattactcacagccatgtatatcttggacagagtttcagatggaatgacttgtctgaaatttgtaaagcttaatataggttttgggggaattattttaatattcaaagaatgttttattatagtcctttgtgttaaaatttagccttactaattataacaataactcataaagttctaaattcagaaggaatgtctgttctttatcaagtgtatgtaactattttttagaaatgccatctactttctagaaacactaaagttattgttttctaagttaaataactataatttatatatctattaaaaaggtacttctcttcccaaaaaaaaaaaaaaaaaaa

SEQ ID NO: 43 - DNA, 854 bp, Homo sapiens
  misc_feature: Incyte ID No 3046106CT1
ttttgaagtatttttaaaaggggtttggaggtagcatccgaaatcatataaagattggggataaatgttgaatttttgagatatggaatgtctattaagaggtggaataaagattgtatgtgtcatactctttggaggaaagtggtcccccaaaatgacagcaattcctaaggagtttgtgaaggggtacatgttggaatcatatagagtaaatatcataaaaactatccatacattactgttgcattggcaagagcacatcatttagaatatacatccaattattaaatttatttaataggcaagatgttatagagaagacagttctcaagattctttttcagtttccattgactaaatttctaactttagaaagctctgaatgtgacatatttcgccattcttcagcaagagtgatgtcaaacttacatccccactttgcaaaaatatatcacttcaatggaggtggcatataaacctgaatttttattttatggaaggttgctatgtgaatatacagagctgaaggtttaggagggcaactaagggtcttatcgtaccacatctctggcccttattgaatgtttcttttcctaagtccattcctgactccagtttgctgtataatcctgagactcctttacagaatacggggatctaacatgtagagactattcctgtaattggtgtttcttggaggcattgcaaaaccaaatttttctttactttgtagcacttttgactaatgttatctaaggactgtatcaaagaattggtttctattagattttagtttaagaaatcttacaattttgttacagagcaggctatttggaggatgaaactgaaattaa

SEQ ID NO: 44 - DNA, 714 bp, Homo sapiens
  misc_feature: Incyte ID No 3012947CB1
accctttcagtaatcattcaaccaacgcttccatgtctctactctgtcgtaacaaaggctgtgggcagcactttgaccctaataccaaccttcctggtcagagttgcctctgaagctgctgccgctaaatatatcccaagccctggaaatggcattggaacagaaggaattagaccaggaacctggggcaggacttgacagtctgatccggactggttccagctgccagaacccaggatgtgatgctgtttaccaaggccctgagagtgatgctactccatgtacctaccacccaggagcaccccgattccatgaggggatgaagtcttggagctgttgtggcatccagaccctggattttggggcattcttggcacaaccagggtgcagagtcggtagacatgactgggggaagcagctcccagcatcttgccgccatgattggcaccagacagattccttagtagtggtgactgtatatggccagattccacttcctgcgtttaactgggtgaaggccagtcaaactgagcttcatgtccacattgtctttgatggtaaccgtgtgttccaagcacagatgaagctctggggggtaagtgaagaccaggggacacaagagtgggaggcagatgggtgaaagagcggctagactggaatagagggtgtcttgagggaaggagttgtactaggaaaatggaggttttctcttca

SEQ ID NO: 45 - DNA, 1434 bp, Homo sapiens
  misc_feature: Incyte ID No 466761CT1
caagaatgtatcctttcagctctctttggttatacctgaagccaggagcgttgagttattagccttgtgtttatattcctctcactgtaattggtgtcattttcccagcagtcctagcagtcctcaagcaagtgggaaatcggaaaagaaaaggacaggcattgtagggaagcagaggataaagaatttagccaacaaaagaaacaatctagtcaatctgggtgcttttatttcctgggtactctctaaacatggctcagagctggtgtagatgaagtaggtgaaacctctgaaaagagtctagaaggcagtagagcaagtcccagaccagaaacatgctcatcttttcatcgtaatgtgccactcggtactatttggtaatgtcactctatttttcctaatcccatcctttggtttgtatttcatatttgtatataaggcaccattttctaaaaatatgactagggtgtgacctaaggttttattctgtgaagatgagtaactggaaagaagctaacactgcagtgggaaggaaggaagagagttgtccaggtggtagttcgacgtgttttgaatctagtccttcctacatggaggataaaagctcctaaagtccactctgggtttgtgattttaatagaaatagaaagggaaactatagaccaatggagatgaaaatcaggggctatcgacagatggaggagaaataaggtgctacatagagaaaggaagagggcagaaggctttcccttcccaaactgggtgagctggggaagccttggttcaggagagtggcactgcccacaactgctttgtgggttgtgcacttccagccgcactctccccctccagttgctgccttcagagccgtactgaagcacgagcttcaataagacaagcacacttcatagtgagagggcagcggtaccaaagcctttcagagagactatggattagacagaaatgatttgtgagaggaagctggagtgaacagcatgaacagcgagtgttacctgacagaggcaagacagctagaagtggcttcagatttagaaacagctgaggggagcaaagacggactgtgtacacagggagggaggatgtctatgggcagagcccttggtgagtatcatcaccaagaaaggcagtccagagtagagatcagccgaatatggaggctgaggtctgtagaactgggccagagaggaccttactgccttagtagcataagggtctggaaaagaagtttctatctcacaacaaaggaaaaagtgaaaagcaaggtggaacttgaagatacgtcacgaaaatcactataaaagtctgatttatgtgtgatgtcaaatcaaactgaaatgaagaatgagattgagtatatctgtggtgactgacctctgtatactagaaacctcaacatctctagaagaggaaata

SEQ ID NO: 46 - DNA, 2298 bp, Homo sapiens
  misc_feature: Incyte ID No 1644171CT1
tgagaaccaactcattttggtatttttagtagagacgaaaccccatcctcccaaagtgctgggattacaggcatgagctgccgcacccggcctccacctgggttttgagccaatcccctggacttgctcctggtttcctcaaggggtggggcagtggtttaggacactcgacaactaagaacaggagttcccaggaaggacaaggatctgcatcccccactgccacttctctgatgtgttcctcaaagctggctcgagggctcgatcccttcatcggactcaggaggggactggttggtgtatccaggtaatttactcttggaagtgactgtagtgaaggtcgtggaagggctcagagggttaattggtttgcagtgcgtctttgtctattgcatgtcttggaaaactcagatcccaaaggcgctgggtttcagagaggacagtggagaccttgctccttttccttaggccgccagtctctcaaatttcagaggaggctgtttccacaactcccctatggaaacacttggcagcggagttgctcctttgcagtttccacaccatggcttttcctttcctttcttctccattccctgatgcatcaacacttacttggagcaatttcctaggagtcagaaccagcaccagccactcggtgtcggtggccaccaaggcttaacattgaccttcccgcctgaccttgatgcagatgtccactgaacacaccgcaggaaagccagggccttcaataccaataagtgtgaatatgtgtgtatgttgtccaagagagattagggagatcacatagactctagggagtagagaacttgtaacagtcttgcaaggctagcatgcacggctccacagcaggtggtggggagcagaggggcaggacctgcagggaagaagcagcctttggatggtgaaatgtgcatggtgcacagtctgtgcatgcccaggagacccagcccgggctgcctcgaggggctcctttgtacacagccagccgcttctcttgggaacaagctgtcctgggggccttacccacgaggcaggagtcaggatgcaccagctcagcaccaggaagtcatcctggacccaggacagtggaaaggcaggcagagggagaggcactctgaggtcaggcagggtaagccagttggcagtcaggttaggtctatgaggagaacctcgagttaggaattcccggttctcagaattgttatcactctggtgcatgctgtcacaggggccgttgcgtttggctttgtggagggcctggacccttccacaagaacacccgaggttccagggcactcaggacaatgtttccaaggaacgagtcgaccaggaaagaacagtgagttctgcaaggggcatccacggagcctgtgataggggctgatgagatggaatctgtcctggacttttcttctcattaaccaccctccgcaaaccccagaacccctcgcctcatctctgtactgtctgccctcttgggggatgggccctcccactttcccctgcctgctcctccatgctgtgagctgctttggcagatctgtttttctgtgtagtcaggggaaaaacaaaaaaagatgcacaactgtgtgggcattgtcatagctgttggtgtcaccactgctttgggggaaatggctgggatgaggctaatacattcatgcaatatttatattttcagggggctgcgttatcagcatgctctccctgccttgggcttttctttccgtcatgttttccttttcgtgttccttctctgatttctcttgtctctgctgctcacaggcctgcccatcagtcagtacagatactcagtgtctggtttctggccagctccgtggagggggctttaagcagaattctgactctttggggtgggggattaggaactgggggaaacttaatgatccagagattcccccaagaggagtgtctggaaggatctgtgcctggacagtggcagaacctttccagtgttcttttggttctgatttcatcagtctcaataaagttccgatctctctttaaaaaaaaaaaaaaacaaaaaaaaaaaaaaaaaaaaaaaaaaaagacaaaaaaaaaaaagggggccccccaaaaggggggggaccccgccccaagcgcgaaagcgcctcaanagctttcccnngaaaaaatttttccccccccaaaattccagcccgctggtggagtcgcctgtcnnnnnnnnnnnnnnnnnnnnnnnctnnnnnnnnnnnnnnnnnnnnnnnnnggnnncnnnnnnnnnnnnnnnnnccc

SEQ ID NO: 47 - DNA, 728 bp, Homo sapiens
  misc_feature: Incyte ID No 3009806CB1
gacaatagggagaatggagaacgtggaggtcttcaccgctgagggcaaaggaaggggtctgaaggccaccaaggagttctgggctgcagatatcatctttgctgagcgggcttattccgcagtggtttttgacagccttgttaattttgtgtgccacacctgcttcaagaggcaggagaagctccatcgctgtgggcagtgcaagtttgcccattactgcgaccgcacctgccagaaggatgcttggctgaaccacaagaatgaatgttcggccatcaagagatatgggaaggtgcccaatgagaacatcaggctggcggcgcgcatcatgtggagggtggagagagaaggcaccgggctcacggagggctgcctggtgtccgtggacgacttgcagaaccacgtggagcactttggggaggaggagcagaaggacctgcgggtggacgtggacacattcttgcagtactggccggcgcagagccagcagttcagcatgcagtacatctcgcacatcttcggagtgattaactgcaacggttttactctcagtgatcagagaggcctgcacagcgtggggcgtaaggatctttccccacctggggctggtgaaccatgactgttggcccaactgtaactggcaaattttacaatgggcatcctgagggcattgaaatcccaaggttcattaccaagattgggaatttgagcctccgggccccttaggg

SEQ ID NO: 48 - DNA, 1158 bp, Homo sapiens
  misc_feature: Incyte ID No 5578191CB1
cagctcgagggacggcaccatggaggactccgaggcggtgcagagggccacagcgctcatcgagcagcggctggcacaggaggaggagaatgagaaactccgaggagacacacgccagaagctgcccatggacttgctggtgctggaggatgagaagcaccacggggctcagagtgcagccctgcagaaggtgaagggccaagagcgcgtgcgcaagacgtccctggacctgcggcgggagatcatcgatgtgggcgggatccagaacctcatcgagctgcggaagaaacgcaagcagaagaagcgggacgctctggccgcctcgcatgagccgcccccagagcccgaggagatcactggccctgtggatgaggagaccttcctgaaagctgcggtggaggggaaaatgaaggtcattgagaagttcctggctgacggggggtcagccgacacgtgcgaccagttccgtcggacagcactgcaccgagcttccctggaaggccacatggaaatcctggagaagcttctagataatggggccactgtggacttccaggatcggctggactgcacagccatgcattgggcctgccgcgggggccacttagaggtggtgaaacttctgcaaagccatggagcagacaccaatgtgagggataagctgctgagcaccccgctgcacgtggcagtccggacagggcaggtggagattgtggagcactttctatccctgggcctggaaatcaatgccagagacagggaaggggatactgccctgcatgacgctgtgaggctcaaccgctacaaaatcatcaaactgctgctcctgcatggggctgacatgatgaccaagaacctggcaggaaagaccccgacggacctggtgcagctctggcaggctgatacccggcacgccctggagcatcctgagccgggggctgagcataacgggctggaggggcctaatgatagtgggcgagagacccctcagcctgtgccagcccagtgaatgcgtgccccagcccagccagctacccagcccctctctgtgtgcagccggagggtcctaagaatggctcccggagctaactgagggcccagccttttttctgcatgatccaggagcacataccacaaactaccacaataaaaaagctg

SEQ ID NO: 49 - protein, 70 aa, Homo sapiens
  misc_feature: Incyte ID No 3601719CD1
MLEPSRQISIFQWEPFGQEQVNPPEEKNVLLKWRRVFLPPRMRRRSQFQERRNFQDLQSIYRKSRILKVN

SEQ ID NO: 50 - protein, 552 aa, Homo sapiens
  misc_feature: Incyte ID No 3445829CD1
MSTFGYRRGLSKYESIDEDELLASLSAEELKELERELEDIEPDRNLPVGLRQKSLTEKTPTGTFSREALMAYWEKESQKLLEKERLGECGKVAEDKEESEEELIFTESNSEVSEEVYTEEEEEESQEEEEEEDSDEEERTIETAKGINGTVNYDSVNSDNSKPKIFKSQIENINLTNGSNGRNTESPAAIHPCGNPTVIEDALDKIKSNDPDTTEVNLNNIENITTQTLTRFAEALKDNTVVKTFSLANTHADDSAAMAIAEMLKVNEHITNVNVESNFITGKGILAIMRALQHNTVLTELRFHNQRHIMGSQVEMEIVKLLKENTTLLRLGYHFELPGPRMSMTSILTRNMDKQRQKRLQEQKQQEGYDGGPNLRTKVWQRGTPSSSPYVSPRHSPWSSPKLPKKVQTVRSRPLSPVATPPPPPPPPPPPPPSSQRLPPPPPPPPPPLPEKKLITRNIAEVIKQQESAQRALQNGQKKKKGKKVKKQPNSILKEIKNSLRSVQEKKMEDSSRPSTPQRSAHENLMEAIRGSSIKQLKRVSNQRTDIGAQIK

SEQ ID NO: 51 - protein, 260 aa, Homo sapiens
  misc_feature: Incyte ID No 2837330CD1
MSLLWTPKGKMRLQAEKLNKAPQGGIGTAAVRPKSLAISSSLVSDVVRPKTQGTDLKTSSHPEMLHGMAPQQKHGQQYKTKSSYKAFAAFPTNTLLLEQKTPTTLPRAAGRETKYANLSSPTSTVSESQLTKPGVIRPVPVKSRILLKKEEEVYEPNPFSKYLEDNSDLFSEQDVTVPPKPVSLHPLYQTKLYPPAKSLLHPQTLSHADCLAPGPFSHLSFSLSDEQENSHTLLSHNACNKLSHPMVAIPEHEALDSKEQ

SEQ ID NO: 52 - protein, 364 aa, Homo sapiens [sequence omitted]

SEQ ID NO: 53 - protein, 527 aa, Homo sapiens [sequence omitted]

SEQ ID NO: 54 - protein, 82 aa, Homo sapiens
  misc_feature: Incyte ID No 5449893CD1
MSQAGAQEAPIKKKRPPVKEEDLKGARGNLTKNQEIKSKTYQVMRECEQAGSAAPSVFSRTRTGTETVFEKPKAGPTKSVFG

SEQ ID NO: 55 - protein, 302 aa, Homo sapiens
  misc_feature: Incyte ID No 282977CD1
MNVQPCSRCGYGVYPAEKISCIDQIWHKACFHCEVCKMMLSVNNFVSHQKKPYCHAHNPKNNTFTSVYHTPLNLNVRTFPEAISGIHDQEDGEQCKSVFHWDMKSKDKEGAPNRQPLANERAYWTGYGEGNAWCPGALPDPEIVRMVEARKSLGEEYTEDYEQPRGKGSFPAMITPAYQRAKKANQLASQVEYKRGHDERISRFSTVADTPELLRSKAGAQLQSDVRYTEDYEQQRGKGSFPAMITPAYQIAKRANELASDVRYHQQYQKEMRGMAGPAIGAEGILTRECADQYGHGYPEEY

SEQ ID NO: 56 - protein, 193 aa, Homo sapiens
  misc_feature: Incyte ID No 3178454CD1
MNTSFSDIELLEDSGIPTEAFLASCCAVVPVLDKLGPTVFAPVKMDLVENIKKVNQKYITNKEEFTTLQKIVLHEVEADVAQVRNSATEALLWLKRGLKFLKGFLTEVKNGEKDIQTALNNAYGKTLRQHHGWVVRGVFALALRATPSYEDFVAALTVKEGDHRKEAFSIGMQRDLSLYLPAMKKQMAILDAL

SEQ ID NO: 57 - protein, 174 aa, Homo sapiens [sequence omitted]

SEQ ID NO: 58 - protein, 230 aa, Homo sapiens [sequence omitted]

SEQ ID NO: 59 - protein, 915 aa, Homo sapiens
  misc_feature: Incyte ID No 4151935CD1
MPLFEAEEGVLSRTQIFPTTIKVIDPEFLEEPPALAFLYKDLYEEAVGEKKKEEETASEGDSVNSEASFPSRNSDTDDGTGIYFEKYILKDDILHDTSLTQKDQGQGLEEKRVGKDDSYQPIAAEGEIWGKFGTICREKSLEEQKGVYGEGESVDHVETVGNVAMQKKAPITEDVRVATQKISYAVPFEDTHHVLERADEAGSHGNEVGNASPEVNLNVPVQVSFPEEEFASGATHVQETSLEEPKILVPPEPSEERLRNSPVQDEYEFTESLHNEVVPQDILSEELSSESTPEDVLSQGKESFEHISENEFASEAEQSTPAEQKELGSERKEEDQLSSEVVTEKAQKELKKSQIDTYCYTCKCPISATDKVFGTHKDHEVSTLDTAISAVKVQLAEFLENLQEKSLRIEAFVSEIESFFNTIEENCSKNEKRLEEQNEEMMKKVLAQYDEKAQSFEEVKKKKMEFLHEQMVHFLQSMDTAKDTLETIVREAEELDEAVFLTSFEEINERLLSAMESTASLEKMPAAFSLFEHYDDSSARSDQMLKQVAVPQPPRLEPQEPNSATSTTIAVYWSMNKEDVIDSFQVYCMEEPQDDQEVNELVEEYRLTVKESYCIFEDLEPDRCYQVWVMAVNFTGCSLPSERAIFRTAPSTPVIRAEDCTVCWNTATIRWRPTTPEATETYTLEYCRQHSPEGEGLRSFSGIKGLQLKVNLQPNDNYFFYVRAINAFGTSEQSEAALISTRGTRFLLLRETAHPALHISSSGTVISFGERRRLTEIPSVLGEELPSCGQHYWETTVTDCPAYRLGICSSSAVQAGALGQGETSWYMHCSEPQRYTFFYSGIVSDVHVTERPARVGILLDYNNQRLIFINAESEQLLFIIRHRFNEGVHPAFALEKPGKCTLHLGIEPPDSVRHK

SEQ ID NO: 60 - protein, 163 aa, Homo sapiens
  misc_feature: Incyte ID No 3012947CD1
MALEQKELDQEPGAGLDSLIRTGSSCQNPGCDAVYQGPESDATPCTYHPGAPRFHEGMKSWSCCGIQTLDFGAFLAQPGCRVGRHDWGKQLPASCRHDWHQTDSLVVVTVYGQIPLPAFNWVKASQTELHVHIVFDGNRVFQAQMKLWGVSEDQGTQEWEADG

SEQ ID NO: 61 - protein, 201 aa, Homo sapiens
  misc_feature: Incyte ID No 3009806CD1
MENVEVFTAEGKGRGLKATKEFWAADIIFAERAYSAVVFDSLVNFVCHTCFKRQEKLHRCGQCKFAHYCDRTCQKDAWLNHKNECSAIKRYGKVPNENIRLAARIMWRVEREGTGLTEGCLVSVDDLQNHVEHFGEEEQKDLRVDVDTFLQYWPAQSQQFSMQYISHIFGVINCNGFTLSDQRGLHSVGRKDLSPPGAGEP

SEQ ID NO: 62 - protein, 329 aa, Homo sapiens
  misc_feature: Incyte ID No 5578191CD1
MEDSEAVQRATALIEQRLAQEEENEKLRGDTRQKLPMDLLVLEDEKHHGAQSAALQKVKGQERVRKTSLDLRREIIDVGGIQNLIELRKKRKQKKRDALAASHEPPPEPEEITGPVDEETFLKAAVEGKMKVIEKFLADGGSADTCDQFRRTALHRASLEGHMEILEKLLDNGATVDFQDRLDCTAMHWACRGGHLEVVKLLQSHGADTNVRDKLLSTPLHVAVRTGQVEIVEHFLSLGLEINARDREGDTALHDAVRLNRYKIIKLLLLHGADMMTKNLAGKTPTDLVQLWQADTRHALEHPEPGAEHNGLEGPNDSGRETPQPVPAQ